(19) United States
(12) Patent Application Publication
(10) Pub. No.: US 2007/0004036 A1
(43) Pub. Date: Jan. 4, 2007

(54) Methods and compositions for keratinocyte culture

(76) Inventors: Rodolfo Faudoa, San Antonio, TX (US); Maria L. Medina, San Antonio, TX (US)

Correspondence Address: WILSON SONSINI GOODRICH & ROSATI, 650 PAGE MILL ROAD, PALO ALTO, CA, 94304-1050, US

(21) Appl. No.: 11/257,573

(22) Filed: Oct. 24, 2005

Related U.S. Application Data
(60) Provisional application No. 60/695,956, filed on Jul. 1, 2005.

(51) Int. Cl. C12N 5/08; C12N 5/06; G06Q 99/00

(52) U.S. Cl. 435/325; 435/404; 435/366; 705/1

(57) ABSTRACT

The invention encompasses composition and methods for cell culture and for therapeutic and cosmetic use. The compositions and methods utilize collagenase, e.g., bacterial collagenase, other isolated collagenase, or synthetic collagenase, e.g., recombinant collagenase. One form of collagenase that can be used in some embodiments of the invention is matrix metalloproteinase-1. The compositions and methods of the invention also optionally utilize a cAMP-elevating agent.

### CROSS REFERENCE

This application claims the benefit of U.S. Provisional Application No. 60/695,956, filed Jul. 1, 2005, which is incorporated herein by reference in its entirety.

### BACKGROUND OF THE INVENTION

Two decades ago, the emergence of the biotechnology industry sparked the development of methods for large-scale cell culture, as companies raced to produce therapeutic quantities of the first recombinant proteins. Continuous refinements to cell culture techniques, instrumentation, and quality control measures ultimately have made large-scale cell culture more a science than an art. Cell culture is vital no only to the development of new pharmaceuticals, but to the testing of existing therapeutics, the advancement of tissue engineering and transplant, and the rigorous understanding of biologic and physiologic systems, including cancer.

There are a number of growth limitations for normal cells grown in culture. To circumvent these limitations, researchers and biotechnology manufacturers often employ ‘immortalized’ (i.e. genetically transformed/mutated) cell lines for the development, testing and manufacturing of therapeutics that will eventually be used to target ‘normal’ human cells. There are many benefits of cell lines such as cost, reproducibility, and longevity in culture. The one main problem is that cell lines are not normal, but simply attempt to simulate normal cell function. This may account for some of the side effects encountered by humans when taking therapeutics developed and tested using cell lines.

Normal cells do not grow ‘ideally’ in traditional culture media. Therefore, technology changed the cell to fit the growth medium. There is still a need to change the medium to fit the cell. For many applications, e.g., the use of keratinocyte culture for research and/or therapeutic purposes, cell lines are not adequate, and current methods for culture often require use of serum, or produce cell cultures that are mixtures of the desired cell type, e.g., keratinocytes, and fibroblasts. Cultures of human cells are increasingly being used in examinations of normal cell, tissue, and organ function and disease, and as in vitro models for toxicology. Successful culture of cells, especially primary culture, often proves to be less than optimal. For example, samples cells obtained from tissue for use in primary culture almost inevitably contain fibroblasts as well as the desired cell type. The fibroblasts generally are from connective tissue, which is found in virtually every organ and tissue. The primary cell culture produced from such a cell sample often is contaminated, to a greater or lesser degree, with fibroblasts that grow and proliferate along with the desired cells, often more quickly and with less fastidious needs in terms of nutrients. For example, keratinocytes from skin explants were rapidly overgrown by less fastidious and faster-growing fibroblasts that were also resident in the tissue. Thus, there has been substantial work expended in the attempt to formulate culture media favoring the selection and successful in vitro cultivation of human cells.

In addition, therapeutics for conditions in which cell growth may be less than ideal, e.g., in wound healing (acute wounds, chronic wounds, burns, and the like) and in cancer, the ability to selectively enhance and/or inhibit cell growth and proliferation has obvious benefits. At present there is a lack of satisfactory methods to enhance and/or inhibit cell growth to optimize such processes as cell culture, wound healing, cancer, and the like. The present invention addresses this lack.

### SUMMARY OF THE INVENTION

In one aspect the invention provides compositions. In some embodiments, the invention provides a cell culture medium containing keratinocyte culture medium, collagenase and a cyclic adenosine monophosphate (cAMP)-elevating agent, where the collagenase and the cAMP-elevating agent are not produced by cells growing in the culture medium. The collagenase can be low-endotoxin collagenase. In some embodiments, the compositions include a cell culture medium containing keratinocyte culture medium and matrix metalloprotease (MMP), where the MMP is not produced by cells in the medium, and where the MMP is MMP-1, MMP-2, MMP-8, or MMP-9. In some embodiments, the MMP is MMP-1, e.g., recombinant MMP-1. In some embodiments, the MMP-1 is present at a concentration of about between about 0.1 ug/ml and 10 ug/ml, or about 1 ug/ml and 3 ug/ml. In some embodiments, the medium containing a MMP further contains a cAMP-elevating agent, e.g., a cAMP-elevating agent that increases intracellular cAMP levels through interactions with cellular G-proteins, and/or through inhibition of a cAMP phosphodiesterase. In some embodiments, the cAMP-elevating agent is forskolin, cholera toxin, dibutyryl cAMP, isobutylmethylxanthine, theophylline, isoproterenol, or PGE2. In some embodiments, the cAMP-elevating agent is forskolin. In some embodiments, the forskolin is at a concentration of between about 0.1 ug/mil and 10 ug/ml. In some embodiments containing MMP-1 and forskolin, the MMP-1 is at a concentration of between about 0.1 ug/ml and 10 ug/ml and the forskolin is at a concentration of between about 0.1 ug/ml and 20 ug/ml. In some embodiments, the medium is serum-free, optionally containing bovine pituitary extract, e.g., at about 10-15 ug/ml. In some embodiments, the medium is animal-protein-free. In some embodiments, the medium is a defined medium. In some embodiments, the medium further contains insulin, transferrin, and hydrocortisone. In some of these embodiments the MMP-1 is at a concentration of between about 0.1 ug/ml to 10 ug/ml and the forskolin is at a concentration of between about 0.1 ug/ml and 20 ug/ml. In some of these embodiments the MMP-1 is at a concentration of about 1 to 5 ug/ml and the forskolin is at a concentration of about 0.8-2.0 ug/ml. In some embodiments, the MMP-1 is at a concentration of about 1.5-2.0 ug/ml the forskolin is at a concentration of about 1.5-2.0 ug/ml, the insulin is at a concentration of about 2-15 ug/ml, the transferrin is at a concentration of about 5-15 ug/ml, and the hydrocortisone is at a concentration of about 0.02-0.5 ug/ml.

In some embodiments, the invention provides a cell culture medium containing keratinocyte culture medium and collagenase, where the collagenase is not produced by cells growing in the medium, and where the collagenase is low-endotoxin collagenase. In embodiments containing collagenase, the collagenase may be present at a concentration of, e.g., about 0.1 to 10 ug/ml. In embodiments containing collagenase, the collagenase may be present at a concentration of, e.g., about 0.0001 to 0.05 U/ml. In some embodiments containing collagenase, the collagenase is bacterial collagenase, such as collagenase isolated from Clostridium histolyticum. In some embodiments containing collagenase, the collagenase contains collagenase I. In some embodiments containing collagenase, the collagenase contains collagenase II. In some embodiments containing collagenase, the collagenase is highly purified. In some embodiments, the medium is 10× concentrated.

In some embodiments, the invention provides a keratinocyte culture medium containing collagenase and forskolin, where neither the collagenase nor the forskolin is produced by cells in the culture medium. The medium may further contain insulin, transferrin, and hydrocortisone. In some embodiments, the collagenase is collagenase isolated from Clostridium histolyticum. In some embodiments, the collagenase is present at about 2-3.5 ug/ml and the forskolin is present at about 1.5-2.5 ug/ml. In some embodiments, the collagenase is present at about 2-3.5 ug/ml and the forskolin is present at about 1.5-2.5 ug/ml. In some embodiments containing insulin, transferrin, and hydrocortisone, the insulin is present at about 5 ug/ml, the transferrin is present at about 10 ug/ml, and the hydrocortisone is present at about 0.1-0.2 ug/ml. In some embodiments, the medium further contains bovine pituitary extract at a concentration of about 10-15 ug/ml.

In some embodiments, the invention provides a keratinocyte culture medium comprising a peptide that is at least about 80% identical to SEQ ID NO:1, 2, or 3, wherein the peptide is not produced by cells in the medium. The peptide can be at a concentration of, e.g., about 0.1-10.0 ug/ml, or 0.5-5.0 ug/ml In some embodiments the medium further contains a cAMP-elevating agent.

In some embodiments, the invention provides a kit for keratinocyte culture containing a first container that contains isolated collagenase and a second container containing a cAMP-elevating agent. In some embodiments the first and second containers are the same. In some embodiments, the collagenase is a MMP, e.g., MMP-1. In some embodiments, the isolated collagenase is in cell culture medium. In some embodiments, the isolated collagenase is in a supplement for addition to a cell culture medium. In some embodiments, the supplement is a concentrated cell medium composition. In some embodiments, the supplement is a solid composition. The kit may further contain a keratinocyte to be cultured and/or instructions.

In some embodiments, the invention provides a method of preparing a keratinocyte culture medium by adding isolated MMP-1 to a base keratinocyte culture medium. In some embodiments the method further includes adding to the medium a cAMP-elevating agent.

In some embodiments, the invention provides a method of culturing a keratinocyte by contacting the keratinocyte with a keratinocyte culture medium that contains MMP-1, where the MMP-1 is not produced by cells in the medium, and culturing the keratinocyte under conditions suitable to support culture of the keratinocyte. In some embodiments, the medium further contains a cAMP-elevating agent.

In some embodiments, the invention provides a method for producing a keratinocyte product by culturing keratinocytes in the cell culture medium of claim 1 or 2 until said product accumulates; and recovering said product. The method can include purifying said product.

In some embodiments, the invention provides a business method for the marketing and sale of keratinocyte culture media by supplying a keratinocyte cell culture composition containing isolated collagenase and a cAMP-elevating agent to a customer, and receiving payment for the composition.

In some embodiments, the invention provides a method of culturing keratinocytes by contacting growing keratinocytes with a culture medium containing collagenase, where the collagenase is not produced by cells in the medium, and where the collagenase is low-endotoxin collagenase. In some embodiments, the collagenase contains isolated MMP-1, e.g., recombinant MMP-1. In some embodiments, the culture medium further contains a cyclic adenosine monophosphate (cAMP)-elevating agent, where the cAMP-elevating agent is not produced by cells in the medium. In some of these embodiments, the cAMP-elevating agent is selected from the group consisting of forskolin, cholera toxin, dibutyryl cAMP, isobutylmethylxanthine, theophylline, isoproterenol, and PGE2. In some embodiments, the cAMP-elevating agent is forskolin. In some embodiments, the MMP-1 is at a concentration of between about 0.5 ug/ml and 5 ug/ml in the culture medium and the forskolin is present at a concentration of about 0.8 ug/ml to about 5 ug/ml. In some embodiments, the growing keratinocytes are fetal keratinocytes. In some embodiments, the culture medium is partially or completely replaced periodically. In some embodiments the culture medium is replaced periodically until a cell culture is produced where the cells of the cell culture comprise greater than about 99.9% keratinocytes. In some embodiments, the culture medium is partially or completely replaced about every day. In some embodiments, the keratinocytes are cultured until a cell culture is produced where the cells of the cell culture contain greater than about 99% keratinocytes. In some embodiments, the method includes passing the keratinocytes when they are about 60-70% confluent.

In some embodiments, the invention provides a keratinocyte culture that persists for more than about one year, where the cells of the culture contain at least about 99% keratinocytes, and where the cells are contacted with cell culture medium comprising collagenase, where the collagenase is not produced by cells in the medium.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a graph illustrating the shelf life of defined and undefined media, with and without collagenase,

FIG. 2 presents a graph illustrating keratinocyte survival in depleted media, either supplemented or not supplemented with collagenase.

FIG. 3 presents a graph illustrating population doublings of keratinocytes grown in defined media with and without collagenase and forskolin.

FIG. 4 presents a graph illustrating the growth of chondrocytes in media supplemented with collagenase.

FIG. 5 presents a graph illustrating fibroblast proliferation in media, either supplemented or not supplemented with collagenase.

### DETAILED DESCRIPTION OF THE INVENTION

I. Overview

The invention encompasses methods and compositions for the use of a collagenase, such as matrix metalloprotease-1 (MMP-1) (also referred to herein as “collagenase-1” or “interstitial collagenase”) and cAMP-elevating agents for cell culture and for therapeutic and other purposes. In cell culture embodiments, virtually any type of cell that may be cultured can be advantageously cultured using the methods and compositions of the invention; thus, the invention is useful in, e.g., primary cell culture and cell line culture. The methods of the invention allow long-term culture of cells with low or no contamination by fibroblasts.

Any desired cell type may be cultured in vitro in the presence of one of the culture media of the present invention. Non-exclusive examples of cell types that may be cultured include stem cells, progenitor cells, mesenchymal cells, epithelial cells, such as keratinocytes, cartilaginous cells, osseous cells, muscular cells, gland cells, fat cells, pericytes, satellite cells and dermal cells.

In some embodiments, the type of cells to be cultured is keratinocytes. Where compositions and methods of the invention are used in the culture of keratinocytes, the keratinocytes may be from humans or animals and may be of adult, neonatal, or fetal origin. In some embodiments the keratinocytes are of fetal origin. The compositions and methods of the invention include compositions and methods allowing long-term culture of highly purified keratinocyte cultures, especially fetal keratinocyte cultures.

Forms of collagenase that may be used in the invention include, but are not limited to, collagenase isolated from cells, e.g., from bacterial cells, and synthetic collagenase, e.g., recombinant collagenase. As an illustrative example, the invention is often described in terms of the use of MMP-1, however, it is understood that any suitable collagenase may be used in the methods and compositions of the invention. MMP-1 that may be used in the compositions and methods of the invention include the entire native polypeptide (either in its final form or as a preprotein), as well as analogs, fragments, and modified forms of MMP-1, which are included in the term “MMP-1” as used herein. The MMP-1 may be from any source, including, but not limited to, bacterial, animal, mammalian, or human, and may be of natural origin, synthetic, or recombinant. cAMP elevating agents include PGE2, isoproterenol, forskolin, dibutyryl cAMP, and theophylline. Further cAMP-elevating agents are described herein.

In some embodiments, the cAMP-elevating agent is forskolin.

The therapeutic uses of the compositions of the invention include uses in wound healing, cosmetic uses, and uses in cancer therapeutics.

II. Collagenases

A. Overview

The methods and compositions of the invention utilize a collagenase. In cell culture aspects of the invention, the collagenase is a collagenase that is not produced by cells in the medium. In some embodiments, the collagenase is a partially or highly purified collagenase, e.g. a bacterial collagenase such as a collagenase isolated from Clostridium histolyticum. In some embodiments, collagenases useful in the invention are matrix metalloproteases (MMPS, also called metalloproteinases). Exemplary MMPs useful in methods and compositions of the invention include MMP-1, MMP-2, MMP-8, MMP-9, and MMP-13, In some embodiments, the invention utilizes MMP-1. In addition, functionally active fragments, variants, and analogs of a collagenase, such as MMP-1, are also included within the term “collagenase,” or, in some embodiments, “MMP” or “MMP-1,” as used herein. An agent that induces the cell to increase its production of collagenase, e.g., MMP-1 (herein, a “collagenase-inducing agent” or an “MMP-1-inducing agent”) may also be used in some embodiments.

For convenience, the invention will sometimes be described with reference to MMP-1 as an exemplary collagenase, however, it is understood that any suitable collagenase may be used in the compositions and methods of the invention. MMP-1 is also known as matrix metalloproteinase-1, collagenase-1 and interstitial collagenase. MMP-1 from any source, natural or synthetic, may be used, and the MMP-1 may be the proenzyme or the active enzyme.

Matrix metalloproteinases (MMPs) are a large family of zinc proteinases that are secreted by both resident and inflammatory cells. The MMP family of enzymes contributes to both normal and pathological tissue. MMPs play a key role in the migration of normal and malignant cells. They also act as regulatory molecules, both by functioning in enzyme cascades and by processing matrix proteins, cytokines, growth factors and adhesion molecules to generate fragments with enhanced or reduced biological effects. The MMPs usually degrade multiple substrates, with considerable substrate overlap between individual MMPs. For example, interstitial collagenase (MMP-1) is capable of degrading casein, gelatin, antitrypsin, MBP, Selectin, pro-TNF and IL-1, and pro-MMP-2 and MMP-9. MMP-2 can degrade fibrillar collagen, elastin, IGF-binding proteins, FGF receptor and can activate MMP-1, MMP-9 and MMP-13. Collectively, they are capable of remodeling or degrading virtually all of the molecules of the extracellular matrix. This processing of the extracellular matrix occurs in wound healing and angiogenesis, as well as in development, differentiation, cell migration, and tumor cell metastasis. MMP-1 is important in wound healing because this metalloproteinase has been shown to play important roles in reepithelialization, formation of the provisional matrix, and angiogenesis. The triple helical structure of fibrillar collagen makes it very resistant to proteolysis, and only a very limited number of MMPs, including MMP-1, can cleave it.

MMPs are expressed as latent proenzymes, which must be activated by proteolytic cleavage of the prodomain. A highly conserved cysteine at a constant position in the prodomain, called the cysteine switch, functions in activation. This cysteine has been shown to coordinate with a zinc cation at the active site, thereby preventing hydration of the cation and subsequent proteolytic. Latent forms of MMPs can be activated by a variety of treatments affecting the cysteine. The present invention encompasses both the proenzyme form and the activated enzyme form of MMP-1, and fragments thereof.

Natural sources of MMP-1 include bacteria, rats, and humans, as well as media from cell cultures. One readily available source is crude or purified collagenase from Clostridium histolyticum, available from, e.g., Sigma, Roche, Invitrogen, Worthington Biochemical, SERVA GmbH, and the like. In some embodiments, the crude preparation may be used. However, these crude collagenase preparations are heterogeneous, containing many different enzymes, cellular debris, pigments and endotoxins. As the name implies, the primary enzyme constituent is collagenase, but in some embodiments, standard purification techniques, e.g., affinity chromatography, may be used to produce a more purified molecule and to remove unwanted materials such as endotoxins. Accordingly, in some embodiments, the invention utilizes a collagenase purified from any suitable source, e.g., Clostridium histolyticum. The collagenase may be partially purified, (e.g., a crude preparation) or highly purified, or any suitable grade of purity. In some embodiments, the collagenase is greater than about 10, 20, 30, 40, 50, 60, 70, 80, 90, 95, 96, 97, 98, 99, 99.5, or 99.9% pure. In some embodiments, the collagenase has an activity of greater than about 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 20, 30, 40, 50, or more than 50 U/mg (PZ units). In some embodiments, the collagenase used in the invention has been purified to be largely free from clostripain, trypsin-like proteases and neutral protease. In some embodiments, a highly purified collagenase is used. “Highly purified collagenase,” as used herein, encompasses collagenase that is largely free from clostripain, trypsin-like proteases and neutral protease, and that has an activity of greater than or equal to 3 U/mg (PZ units). The collagenase may contain either or both of collagenase I and/or collagenase II. In some embodiments, the collagenase contains collagenase I. In some embodiments, the collagenase contains collagenase II.

In, e.g., cell culture embodiments, the collagenase is not produced by cells in the medium, although it may have been produced by other cells. As used herein “not produced by cells in the medium,” or similar expressions, refers to collagenase that is in addition to any collagenase produced by cells or other substances in the medium; it does not refer to, e.g., a type of collagenase that is not produced by the cells, but simply to an external source of collagenase. Cells in the medium may, and often do, produce collagenase(s), some of which may be similar to or identical to the collagenase(s) useful in the invention; however, generally, in the context of cell culture, the collagenase of the invention is an externally-added collagenase, whether or not it is a type similar to or identical to collagenase(s) produced by cells in the medium.

Collagenase purified from, e.g., bacterial sources, often contains endotoxins. While any suitable collagenase may be used in embodiments of the invention, including high-endotoxin collagenase, typically endotoxins associated with the collagenase have been partially or completely removed or destroyed. Collagenases in crude preparations often have an endotoxin content of greater than 1000 endotoxin unit (EU)/mg; a collagenase used in the compositions and methods of the invention can have an endotoxin content of less than about 10,000; 5,000; 1000; 900; 800; 700; 600; 500; 400; 300; 200; 100; 90; 80; 70; 60; 50; 40; 30; 10; 5; 4; 3; 2; or 1 EU/mg, or may be essentially free of endotoxins (e.g., if recombinant MMP is used). In some embodiments, a low-endotoxin collagenase is used; a low-endotoxin collagenase contains less than 100 EU/mg endotoxin. In some embodiments, a very low-endotoxin collagenase is used; a very low-endotoxin collagenase contains less than 10 EU/mg endotoxin. In some embodiments, a highly purified, low- or very-low endotoxin collagenase is used. In some embodiments, a collagenase that is essentially free of endotoxins is used. One source of collagenases useful in the invention is SERVA GmbH, Heidelberg, Germany, which offers multiple grades and purities of collagenase, including highly-purified low- and very low-endotoxin collagenase.

Some embodiments of the invention utilize a collagenase that is a matrix metalloprotease (MMP), where the MMP is not MMP produced by cells in the medium. In some embodiments, the MMP is MMP-1, MMP-2, MMP-8, or MMP-9. In some embodiments, the MMP is mammalian MMP. In some embodiments, the mammalian MMP-1. Exemplary types include rat and human MMP-1. MMP-1 can degrade a broad range of substrates including types I, II, VII, VIII, and X collagens as well as casein, gelatin, alpha-1 antitrypsin, myelin basic protein, L-Selectin, pro-TNF, IL1b, IGF-BP3, IGF-BP5, pro MMP-2 and pro MMP-9. A significant role of MMP-1 is the degradation of fibrillar collagens in extracellular matrix remodeling, characterized by the cleavage of the interstitial collagen triple helix into ¾, ¼ fragments. However, as the list of substrates above illustrates, the role of MMP-1 is more diverse than originally envisaged, and may involve enzyme cascades, cytokine regulation and cell surface molecule modulation. MMP-1 is expressed by fibroblasts, keratinocytes, endothelial cells, monocytes and macrophages. Structurally, MMP-1 may be divided into several distinct domains: a pro-domain which is cleaved upon activation, a catalytic domain containing the zinc binding site, a short hinge region and a carboxyl terminal (hemopexin-like) domain. See, e.g., “Interstitial Collagenase” by T. E. Cawston (2004) in Handbook of Proteolytic Enzymes (ed. A. J. Barrett, N. D. Rawlings, J. F. Woessner) pp. 472-480, Academic Press, San Diego, which is incorporated herein in its entirety.

Synthetic MMP-1 may be produced by peptide synthesis or as recombinant MMP-1. In some embodiments, recombinant human MMP-1 (rhMMP-1) is used. Such recombinant MMP-1 may be obtained from, e.g., R&D Systems, Minneapolis.

Matrix metalloprotease-inducing agents can also be useful in embodiments of the invention. These include IL-6, fibronectin fragments, and others known in the art.

B. Sequence

As described, MMP-1 from any source may be used in the invention. In some embodiments, human MMP-1 (e.g., rhMMP-1) is used. The sequence of the pro-protein of human MMP-1 is given in Table 1 (SEQ ID NO: 1). See, e.g., Templeton et al. (1990) Cancer Res. 50:5431-5437, which is incorporated herein by reference.

TABLE 1
Sequence of human MMP-1 proenzyme

MHSFPPLLLLLFWGVVSHSFPATLETQEQDVDLVQKYLEKYYNLKNDGRQ
VEKRRNSGPVVEKLKQMQEFFGLKVTGKPDAETLKVMKQPRCGVPDVAQF
VLTEGNPRWEQTHLTYRIENYTPDLPRADVDHAIEKAFQLWSNVTPLTFT
KVSEGQADIMISFVRGDHRDNSPFDGPGGNLAHAFQPGPGIGGDAHFDED
ERWTNNFREYNLHRVAAHELGHSLGLSHSTDIGALMYPSYTFSGDVQLAQ
DDIDGIQAIYGRSQNPVQPIGPQTPKACDSKLTFDAITTIRGEVMFFKDR
FYMRTNPFYPEVELNFISVFWPQLPNGLEAAYEFADRDEVRFFKGNKYWA
VQGQNVLHGYPKDIYSSFGFPRTVKHIDAALSEENTGKTYFFVANKYWRY
DEYKRSMDPGYPKMIAHDFPGIGHKVDAVFMKDGFFYFFHGTRQYKFDPK
TKRILTLQKANSWFNCRKN

The sequence of the mature proenzyme, produced by enzymatic cleavage of the first 19 amino acids, is given in Table 2 (SEQ ID NO: 2)

TABLE 2
Sequence of the mature MMP-1 proenzyme

FPATLETQEQDVDLVQKYLEKYYNLKNDGRQVEKRRNSGPVVEKLKQMQE
FFGLKVTGKPDAETLKVMKQPRCGVPDVAQFVLTEGNPRWEQTHLTYRIE
NYTPDLPRADVDHAIEKAFQLWSNVTPLTFTKVSEGQADIMISFVRGDHR
DNSPFDGPGGNLAHAFQPGPGIGGDAHFDEDERWTNNFREYNLHRVAAHE
LGHSLGLSHSTDIAGALMYPSYTFSGDVQLAQDDIDGIQAIYGRSQNPVQ
PIGPQTPKACDSKLTFDAITTIRGEVMFFKDRFYMRTNPFYPEVELNFIS
VFWPQLPNGLEAAYEFADRDEVRFFKGNKYWAVQGQNVLHGYPKDIYSSF
GFPRTVKHIDAALSEENTGKTYFFVANKYWRYDEYKRSMDPGYPKMIAHD
FPGIGHKVDAFMKDFFYFFHGTRQYKFDPKTKRILTLQKANSWFNCRKN

The sequence of one form of the activated enzyme, produced by enzymatic cleavage of the N-terminal 81 amino acids of the mature proenzyme, is given in Table 3 (SEQ ID NO: 3).

TABLE 3
Sequence of the activated MMP-1

VLTEGNPRWEQTHLTYRIENYTPDLPRADVDHAIEKAFQLWSNVTPLTFT
KVSEGQADIMISFVRGHHRDNSPFDGPGGNLAHAFQPGPGIGGDAHFDED
ERWTNNFREYNLHRVAAHELGHSLGLSHTDIGALMYPSYTFSGDVQLAQD
DIDGIQAIYGRSQNPVQPIGPQTPKACDSKLTFDAITTIRGEVMFFKDRF
YMRTNPFYPEVELNFISVFWPQLPNGLEAAYEFARDEVRFFKGNKYWAVQ
GQNVLHGYPKDIYSSFGFPRTVKHIDAALSEENTGKTYFFVANKYWRYDE
YKRSMDPGYPKMIAHDFPGIGHKVDAVFMKDGFFYFFHGTRQYKFDPKTK
RILTLQKANSWFNCRKN

Embodiments of the invention may utilize any suitable form of MMP-1, e.g., SEQ ID NO:1, 2, or 3. Some embodiments of the invention utilize a protein of SEQ ID NO: 3.

C. Variants, Analogs, Modifications

The invention also encompasses compositions and methods that utilize functional variants, analogs, and other modifications of a collagenase, e.g., MMP-1, as well as peptide mimetics. As used herein, a “functional” variant, analog, fragment, modified polypeptide, peptide mimetic, and the like, encompasses a polypeptide or molecule that is a variant, analog, fragment, peptide mimetic or modified polypeptide of the native molecule (e.g., human MMP-1) that retains sufficient activity or function to produce the desired effect, either enhanced, unchanged, or decreased, when used in a composition or method of the invention.

Variants: Amino acid sequence variants of the collagenase, e.g., MMP-1, polypeptides of the present invention can be substitutional, insertional or deletion variants. Deletion variants lack one or more residues of the native protein that are not essential for function. Insertional variants typically involve the addition of material at a non-terminal point in the polypeptide. Terminal additions, called fusion proteins, are also encompassed by the invention.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Some embodiments of the invention utilize MMP-1 polypeptides having at least 70%, at least 80%, at least 90%, at least 95% or greater than 95% sequence identity to the amino acid sequence of SEQ ID NO: 1, SEQ ID NO:2, or SEQ ID NO:3.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative MMP-1 variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:1) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present invention includes polypeptides having a conservative amino acid change, compared with an amino acid sequence disclosed herein. For example, variants can be obtained that contain one or more amino acid substitutions of SEQ ID NO:1, 2, or 3, in which an alkyl amino acid is substituted for an alkyl amino acid in a MMP-1 amino acid sequence, an aromatic amino acid is substituted for an aromatic amino acid in a MMP-1 amino acid sequence, a sulfur-containing amino acid is substituted for a sulfur-containing amino acid in a MMP-1 amino acid sequence, a hydroxy-containing amino acid is substituted for a hydroxy-containing amino acid in a MMP-1 amino acid sequence, an acidic amino acid is substituted for an acidic amino acid in a MMP-1 amino acid sequence, a basic amino acid is substituted for a basic amino acid in a MMP-1 amino acid sequence, or a dibasic monocarboxylic amino acid is substituted for a dibasic monocarboxylic amino acid in a MMP-1 amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Particular variants of MMP-1 useful in the compositions and methods of the invention are characterized by having at least about 70%, at least about 80%, at least about 90%, at least about 95%, at least about 96%, at least about 97%, at least about 98%, at least about 99%, or greater than 95%, 96%, 97%, 98%, or 99% sequence identity to the corresponding amino acid sequence (e.g., SEQ ID NO:1, 2, or 3), wherein the variation in amino acid sequence can be due to one or more conservative amino acid substitutions.

Conservative amino acid changes in a collagenase, e.g., MMP-1, gene can be introduced, for example, by substituting nucleotides for the nucleotides that code for the collagenase (e.g., that code for SEQ ID NO: 1, 2, or 3 in the case of MMP-1). Such “conservative amino acid” variants can be obtained by oligonucleotide-directed mutagenesis, linker-scanning mutagenesis, mutagenesis using the polymerase chain reaction, and the like (see, e.g., Ausubel et al. (eds.), Short Protocols in Molecular Biology, 3rd. Edition, (John Wiley & Sons 1995) at pages 8-10 to 8-22; and McPherson (ed.), Directed Mutagenesis: A Practical Approach (IRL Press 1991)).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the invention.

A specialized kind of insertional variant is the fusion protein. It is contemplated that the entire collagenase, e.g., MMP-1, protein or a fragment of the collagenase, e.g., MMP-1, protein may be used to construct a fusion protein to enhance tissue specific or cell specific functions of the collagenase, e.g., MMP-1, protein useful in the invention.

A fusion protein generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

Analogs Analogs of a collagenase, e.g., MMP-1, may also be used in the compositions and methods of the invention. An “analog,” as that term is used herein, encompasses a polypeptide or protein in which one or more amino acids is substituted with an amino acid that is not one of the twenty amino acids coded for by the genetic code. Such an amino acid may be a natural or unnatural amino acid, as described more fully below.

The term “amino acid” as used herein encompasses an organic compound containing both a basic amino group and an acidic carboxyl group. Included within this term are natural amino acids (e.g., L-amino acids), and amino acids which are known to occur biologically in free or combined form but usually do not occur in proteins. Included within this term also are modified and unusual amino acids, such as those disclosed in, for example, Roberts and Vellaccio (1983) The Peptides, 5: 342-429 (e.g., D-amino acids). In addition, the term “amino acid” also includes other non-naturally occurring amino acids besides the D-amino acids, which are functional equivalents of the naturally-occurring amino acids. Such non-naturally-occurring (also referred to herein as “unnatural amino acids”) amino acids include, for example, norleucine (“Nle”), norvaline (“Nva”), β-Alanine, L- or D-naphthalanine, ornithine (“Orn”), homoarginine (homoArg) and others well known in the peptide art, such as those described in M. Bodanzsky, Principles of Peptide Synthesis, 1st and 2nd revised ed., Springer-Verlag, New York, N.Y., 1984 and 1993, and Stewart and Young, Solid Phase Peptide Synthesis, 2nd ed., Pierce Chemical Co., Rockford, Ill., 1984. Amino acids and amino acid analogs can be purchased commercially (Sigma Chemical Co.; Advanced Chemtech; RSP; Bachem; or ChemImpex) or synthesized using methods known in the art.

“Natural amino acids” include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-CBZ-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

Standard three- and one-letter abbreviations for natural amino acid residues or amino acids apply throughout the specification unless otherwise indicated.

Unnatural amino acids that fall within the scope of this invention are by way of example and without limitation, those described in U.S. Provisional Patent Application No. 60/695,956, filed Jul. 1, 2005, which is incorporated by reference herein in its entirety.

“Amino acids residue” has its customary meaning in the art and refers to an amino acid that is part of a peptide or polypeptide chain; “amino acid residue” as used herein also refers to various amino acids where sidechain functional groups are coupled with appropriate protecting groups known to those skilled in the art. “The Peptides”, Vol 3, 3-88 (1981) discloses numerous suitable protecting groups. Examples of amino acids where sidechain functional groups are coupled with appropriate protecting groups include, but are not limited to, Asp(OMe), Glu(OMe), Hyp(OMe), Asp(O^{t} Bu), Glu(O^{t} Bu), Hyp(O^{t} Bu), Thr(O^{t} Bu), Asp(OBzl), Glu(OBzl), Hyp(OBzl), and Thr(OBzl).

Thus, some embodiments of the invention utilize an MMP-1 that contains one or more of the amino acids of the sequence, e.g., SEQ ID NO: 1, 2, or 3, that are substituted with, e.g., one or more of the amino acids described above that are not one of the twenty naturally coded amino acids. Such analogs may contain any number of substitutions, so long as the peptide retains sufficient activity to be functional in the compositions and methods of the invention.

Peptide mimetics Another embodiment for the preparation of polypeptides according to the invention is the use of peptide mimetics. Mimetics are peptide-containing molecules that mimic elements of protein secondary structure. See e.g., Johnson et al., In: Biotechnology And Pharmacy, Pezzuto et al., eds., Chapman and Hall, New York, 1993. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principle outline above, to engineer second generation molecules having many of the natural properties of a collagenase, e.g., MMP-1, useful in the invention, but with altered and even improved characteristics.

D. Fragments

The amino acid sequence for various forms of human MMP-1 is provided in Tables 1, 2, and 3 (SEQ ID NOS:1, 2, and 3). In addition to the entire MMP-1 molecule, embodiments of the present invention also relate to fragments of the polypeptide, and to fragments of other collagenases that may be used in compositions and methods of the invention. Fragments may be generated by genetic engineering of translation stop sites within the coding region, or may be synthesized by chemical means. Alternatively, treatment of the MMP-1 protein with proteolytic enzymes can produces a variety of N-terminal, C-terminal and internal fragments. Polypeptides range from 6, 7, 8, 9, 10, 15, 20, 25, 30, 35, 40, 45, and 50 residues, such as those made synthetically, up to 100, 150, 200, 250, 300, 350, 400 and more residues, which are conveniently produced by recombinant means or by proteolytic digestion of full length MMP-1. One or more fragments may be used in a given composition or method.

In certain embodiments the size of the collagenase, e.g., MMP-1 fragment used in the compositions and/or methods of the invention may comprise, but is not limited to, about 10 to about 450 amino acids, or about 20 to about 400 amino acids, or about 30 to about 350 amino acids, or about 40 to about 300 amino acids, or about 50 to about 250 amino acids, or about 50 to about 200 amino acids, or about 50 to about 150 amino acids, or about 50 to about 100 amino acids, or about 100 to about 450 amino acids, or about, or about 100 to about 400 amino acids, or about 100 to about 350 amino acids, or about 100 to about 300 amino acids, or about 100 to about 250 amino acids, or about 100 to about 200 amino acids, or about 100 to about 150 amino acids, or about 150 to about 450 amino acids, or about 150 to about 400 amino acids, or about 150 to about 350 amino acids, or about 150 to about 300 amino acids, or about 150 to about 300 amino acids, or about 150 to about 250 amino acids, or about 150 to about 200 amino acids, or about 200 to about 450 amino acids, or about 200 to about 400 amino acids, or about 200 to about 350 amino acids, or about 200 to about 300 amino acids, or about 200 to about 250 amino acids, or about 250 to about 450 amino acids, or about 250 to about 450 amino acids, or about 250 to about 400 amino acids, or about 250 to about 350 amino acids, or about 250 to about 300 amino acids, or about 300 to about 450 amino acids, or about 300 to about 400 amino acids, or about 300 to about 350 amino acids, or about 350 to about 450 amino acids, or about 350 to about 400 amino acids, or about 400 to about 450 amino acids.

III. cAMP-Elevating Agents

A. Definition and Examples

In some embodiments, the compositions and methods of the invention utilize, in addition to a collagenase, e.g., MMP-1, a cAMP-elevating agent. As used herein, a “cAMP-elevating agent” encompasses agents that elevate intracellular cAMP levels and/or that augment or potentiate the action of cAMP. See, e.g., U.S. Pat. No. 6,610,535, the disclosure of which is incorporated by reference in its entirety. Any suitable cAMP-elevating agent may be used, including, but not limited to, agents that increase intracellular intracellular cAMP levels through interactions with cellular G-proteins, and agents that increase intracellular cAMP levels through inhibition of a cAMP phosphodiesterase.

Useful in the invention are compounds that may activate adenylate cyclase including, but not limited to: forskolin (FK), cholera toxin (CT), pertussis toxin (PT), prostaglandins (e.g., PGE-1 and PGE-2), colforsin and β-adrenergic receptor agonists. β-Adrenergic receptor agonists include albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, soterenol, salmeterol, terbutaline, tretoquinol, tulobuterol, and xamoterol.

Also useful in the invention are compounds which may inhibit cAMP phosphodiesterase(s), and thereby increase the half-life of cAMP. Such compounds include amrinone, milrinone, xanthine, methylxanthine, anagrelide, cilostamide, medorinone, indolidan, rolipram, 3-isobutyl-1-methylxanthine (IBMX), chelerythrine, cilostazol, glucocorticoids, griseolic acid, etazolate, caffeine, indomethacin, theophylline, papverine, methyl isobutylxanthine (MIX), and fenoxanine.

Certain analogs of cAMP, e.g., which are agonists of cAMP, can also be used. Exemplary cAMP analogs which may be useful in the present method include dibutyryl-cAMP (db-cAMP), (8-(4)-chlorophenylthio)-cAMP (cpt-cAMP), 8-[(4-bromo-2,3-dioxobutyl)thio]-cAMP, 2-[(4-bromo-2,3-dioxobutyl)thio]-cAMP, 8-bromo-cAMP, dioctanoyl-cAMP, Sp-adenosine 3′:5′-cyclic phosphorothioate, 8-piperidino-cAMP, N⁶-phenyl-cAMP, 8-methylamino-cAMP, 8-(6-aminohexyl)amino-cAMP, 2′-deoxy-cAMP, N⁶,2′-O-dibutryl-cAMP, N⁶,2′-O-disuccinyl-cAMP, N⁶-monobutyryl-cAMP, 2′-O-monobutyryl-cAMP, 2′-O-monobutryl-8-bromo-cAMP, N⁶-monobutryl-2′-deoxy-cAMP, and 2′-O-monosuccinyl-cAMP.

In some embodiments, the cAMP-elevating agent is forskolin, cholera toxin, dibutyryl cAMP, isobutylmethylxanthine, theophylline, isoproteronol, or PGE2. In some embodiments, the cAMP-elevating agent is forskolin.

cAMP-elevating agents are available commercially, e.g. from Sigma (St. Louis, Mo.), and may be used at concentrations approximating those described in Green (Proc. Natl. Acad. Sci. USA 15:801-811 (1978)), which is incorporated herein by reference in its entirety. See below for specific examples of concentrations used in compositions and methods of the invention.

B. Modifications

Any of the above-listed compounds useful in the subject methods may be modified to increase the bioavailability, activity, or other pharmacologically relevant property of the compound. For example, forskolin has a formula as shown below:

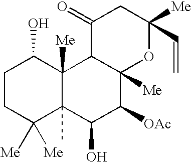

Modifications of forskolin which have been found to increase the hydrophilic character of forskolin without severely attenuating the desired biological activity include acylation of the hydroxyls at C6 and/or C7 (after removal of the acetyl group) with hydrophilic acyl groups. In compounds wherein C6 is acylated with a hydrophilic acyl group, C7 may optionally be deacetylated. Suitable hydrophilic acyl groups include groups having the structure —(CO)(CH₂)ₙX, wherein X is OH or NR₂; R is hydrogen, a C₁-C₄ alkyl group, or two Rs taken together form a ring comprising 3-8 atoms, preferably 5-7 atoms, which may include heteroatoms (e.g., piperazine or morpholine rings); and n is an integer from 1-6, preferably from 1-4, even more preferably from 1-2. Other suitable hydrophilic acyl groups include hydrophilic amino acids or derivatives thereof, such as aspartic acid, glutamic acid, asparagine, glutamine, serine, threonine, tyrosine, etc., including amino acids having a heterocyclic side chain. Forskolin, or other compounds listed above, modified by other possible hydrophilic acyl side chains known to those of skill in the art may be readily synthesized and tested for activity in the present method.

Similarly, variants or derivatives of any of the above-listed compounds may be effective as cAMP agonists in the subject method. Those skilled in the art will readily be able to synthesize and test such derivatives for suitable activity.

In certain embodiments, the subject cAMP agonists can be chosen on the basis of their selectivity for cAMP activation.

In certain embodiments, it may be advantageous to administer two or more of the above cAMP-elevating agents, preferably of different types. For example, use of an adenylate cyclase agonist in conjunction with a cAMP phosphodiesterase antagonist may have an advantageous or synergistic effect.

IV. Cell Culture Compositions and Methods

Cell culture media are used for the culture of a wide range of cell types under varying circumstances and for varying purposes, which may or may not involve the division and multiplication of the cells. Cell culture media of the invention may be employed in conjunction with any suitable culture techniques known or hereinafter to be developed, including batch or continuous culture, perfusion culture, or other techniques, such as those adapted to maximize cell culture, as by the continuous replenishment of nutrients or other media components and continuous removal of cell waste materials.

A. Overview

A striking finding of the invention is that collagenase, e.g., from a bacterial source or recombinant, such as MMP-1, in combination with one or more cAMP-elevating agents, enhances the culture of cells. One major problem in cell culture, especially in primary cell culture, is proliferation and contamination by fibroblasts. In primary cell culture, fibroblasts tend to be sampled along with the cell type desired to be cultured and tend to be passed into the culture. This is because fibroblasts are a major component of connective tissue, a ubiquitous tissue type found in virtually every tissue and organ. The compositions and methods of the present invention encompass the use of a collagenase, e.g., from a bacterial source or recombinant, such as MMP-1, in conjunction with a cAMP-elevating agent, to enhance cell culture by, e.g., reducing the proliferation of fibroblasts. However, simple reduction of fibroblast proliferation is not necessarily the only effect, or even the primary effect of the compositions and methods of the invention. For example, a collagenase, from a bacterial source or recombinant, e.g., MMP-1 can be used to enhance the proliferation of fibroblasts themselves; accordingly fibroblasts are included among the cell types for which the invention is useful. Additionally, compositions and methods of the invention may be used to enhance the culture of cell lines. Hence, decreasing fibroblast contamination is merely one facet of the possible actions of the compositions and methods of the invention. Cell culture may be enhanced by, e.g., increasing the purity of the culture, increasing the time to senescence, increasing the population doublings possible in culture, and the like.

B. Definitions

The term “cell culture medium” or “culture medium” is used herein to encompass any medium in which cells are maintained in vitro in an active and viable state.

By “cell culture” or “culture of cells” or “culture” is meant the maintenance of cells in an artificial, in vitro environment.

“Basal culture medium” or “basal medium” Conventional media, e.g., commercially available media, into which the modulators of the invention (e.g., MMP-1 and/or cAMP-elevating agents) are incorporated for the practice of the invention are herein referred to as “basal culture media”.

“Serum-free” cell culture or cell culture medium refers to culture or culture medium without the use of serum.

“Animal product-free” in the context of cell culture medium, as used herein, encompasses a cell culture medium that does not contain any animal products, i.e., proteins or other compounds or substances of animal origin. A cell culture medium can be “animal product free” if it uses components that are found in animal products, but that are produced by synthetic means. Such components include recombinant proteins, as well as organic and inorganic molecules that may be synthetically produced.

“Animal protein-free” in the context of cell culture medium, as used herein, encompasses a cell culture medium that does not contain any animal protein, i.e., proteins or derivatives of animal origin. A cell culture medium can be “animal protein free” if it uses proteins that are produced by synthetic means, e.g., that are chemically synthesized, or that are produced by recombinant means in non-animal cells.

“Defined cell culture medium,” as used herein, includes cell culture media whose ingredients and proportions are all known. It is understood that a “defined medium” refers to a medium wherein the ingredients added to the medium are known; components of the medium produced by reaction of the ingredients may not be necessarily known in terms of their identity, concentration, or both.

“Exogenously added,” or “exogenous,” as used herein, refers to an ingredient or component that is added to a medium, i.e., that is not an ingredient or component produced during cell culture. An “exogenously added” or “exogenous” ingredient may be an ingredient or component that is the same as a component produced during culture, or it may be an ingredient or component that would not be in the cell culture medium but for being added. For example, “exogenously added collagenase” or “exogenously added MMP-1” refer to collagenase or MMP-1 that are added to the cell culture medium, not collagenase or MMP-1 that are in the cell medium as a result of the culture of cells in that medium, even though such intrinsically-produced collagenase or MMP-1 may also be present in the medium. As used herein, “exogenously added collagenase” or “exogenous collagenase,” or “exogenously added MMP-1” or “exogenous MMP-1,” do not include collagenase or MMP-1 that are part of serum that is added to a cell culture medium, or that are a naturally-occurring component of conditioned medium added to a cell culture medium. As used herein, “exogenously-added” and similar phrases are synonomous with “not produced by cells in the medium,” and similar phrases.

“1×, 10×, 100×, etc.” A “1× formulation, as used herein, refers to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. When more than one ingredient is present, each ingredient in a 1× formulation has a concentration about equal to the concentration of that ingredient in a cell culture medium. The osmolarity and/or pH, however, may differ in a 1× formulation compared to the culture medium, particularly when fewer ingredients are contained in the 1.times.formulation. A “10× formulation,” as used herein, includes a solution where each ingredient in that solution is about 10 times more concentrated than the same ingredient in the cell culture medium. Similarly, “25× formulation,” “50× formulation,” “100× formulation,” “500× formulation,” and “1000× formulation” designate solutions that contain ingredients at about 25-, 50-, 100-, 500-, or 1000-fold concentrations, respectively, as compared to a 1.times.cell culture medium. Again, the osmolarity and pH of the media formulation and concentrated solution may vary.

The compositions and methods of the invention utilize many components and techniques that are known in the art. See, e.g., Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc.; Culture of Animal Cells—A Manual of Basic Technique, Ian Freshney, N.Y., Alan R. Liss, Inc., (1987); and Culture of Epithelial Cells, Freshney, R. I., ed., New York: Wiley-Liss, (1992); all of which are incorporated by reference herein.

C. General Cell Culture

1. Compositions

The invention provides compositions useful in cell culture. These include cell culture media and additives for cell culture media. In some embodiments, the compositions contain an exogenous collagenase. “Exogneous collagenase” is used herein synonymously with “collagenase not produced by cells in the medium,” and similar phrases, described elsewhere herein. In some embodiments the compositions and methods utilize a collagenase, where the collagenase is not produced by cells in the culture medium. In some embodiments, the compositions and methods utilize a highly-purified collagenase. In some embodiments, the composition and methods utilize a low-endotoxin collagenase. In some embodiments the compositions and methods of the invention further include a cAMP-elevating agent, wherein the cAMP-elevating agent is not produced by cells in the medium. As with collagenases, as used herein, a “cAMP-elevating agent that is not produced by cells in the medium,” or similar phrases, is synonomous with “exogenously-added” or “exogenous” cAMP-elevating agent, and refers to cAMP-elevating agent that is in addition to any cAMP-elevating agent produced by cells in the medium. As with collagenases, cells produce cAMP-elevating agents and the cells that are cultured in the medium may produce cAMP-elevating agents that are similar to or identical to the cAMP-elevating agent of the invention, which is added in addition to these cAMP-elevating agents. In some embodiments, the compositions contain an exogenous MMP-1, e.g., a recombinant MMP-1 such as rhMMP-1. In some embodiments, the compositions contain a collagenase, which may be purified from tissue or cellular sources or produced by recombinant or other synthetic means, e.g., exogenous MMP-1, and a cAMP-elevating agent. The cAMP-elevating agent may be exogenously added as a single ingredient or may be a component of a more complex ingredient that is used in the cell culture medium, e.g., serum. The compositions may further comprise other ingredients useful in the culture of cells or of a particular cell type.

a. Basal Cell Culture Media

A cell culture medium of the invention may be produced by addition of one or more ingredients to commercially available stock basal media, or it may be produced “from scratch,” i.e., by adding ingredients or groups of ingredients to a base such as distilled or deionized water.

When cell culture medium is produced by addition of ingredients to commercially available stock basal media, any of the numerous media available may be used. The basal medium employed, as known in the art, contains nutrients essential for supporting growth of the cell under culture, commonly including essential amino acids, fatty acids, and carbohydrates. The medium typically includes additional essential ingredients such as vitamins, cofactors, trace elements, and salts in assimilable quantities. Other biological compounds necessary for the survival/function of the particular cells, such as hormones and antibiotics may also be included. The medium also can include buffers, pH adjusters, pH indicators, and the like.

The choice of basal medium depends in part on the type of final medium desired, i.e., serum-containing, serum-free, animal protein-free, animal product-free, or defined. Exemplary useful media include all known suitable culture media and suitable culture media hereinafter developed which support maintenance and/or growth of the cells therein cultured.

A wide variety of commercially available basal media are available. Stock basal culture media of use in the invention include, but are not limited to, the following: Dulbecco's Modified Eagle Medium (“DMEM”), Iscove modified Dulbeccos' medium; DMEM/F-12 (1:1 DMEM and F-12 vol:vol); Medium 199; F-12 (Ham) Nutrient Mixture; F-10 (Ham) Nutrient Mixture; Minimal Essential Media (MEM), Williams' Media E; Fischer's or Waymouth's MB 752/1, CMRL, Puck's N15 Medium, Puck's N16 Medium; McCoy's 5A Medium, Leibovitz's L15 Medium; ATCC (American Type Culture Collection) CRCM 30; MCDB Media 101, 102, 103, 104; CMRL Media 1066, 1415, 1066, 1415; Roswell Park Memorial Institute Medium (RPMI) 1603, 1634, and 1640; and Hank's or Earl's Balanced Salt Solution. Several versions or modifications of many of these media are available, for example, DMEM 11966, DMEM 10314, MEM 11095, Williams' Media E 12251, Ham F12 11059, MEM-alpha 12561, and Medium-199 11151 (all available from Gibco-BRL/Life Technologies); and MCDB Media developed by Ham, such as MCDB 105, 110, 131, 151, 153, 201, and 302 media. In some embodiments, the basal medium employed is MCDB 153.

The above are merely exemplary, and it is understood that any stock basal medium, suitable for the cell type and application desired, may be used to produce the compositions of the invention. In addition, it will be realized that for optimal results, the basal medium to which the additional ingredient or ingredients is added must be appropriate for the cell type of interest, with key nutrients available at adequate levels to enhance cell growth or product expression. Thus, for example, it may be necessary to increase the level of glucose (or other energy source) in the basal medium, or to add glucose (or other energy source) during the course of culture, if this essential energy source is found to be depleted and to thus limit cell growth or product expression.

If the cell culture media are produced from scratch, standard techniques well-known in the art may be used. See, e.g., Cell Culture Methods for Molecular and Cell Biology, Vol. 1: Methods for Preparation of Media, Supplements, and Substrata for Serum-Free Animal Cell Culture, Barnes, D. W., et al., eds., New York, Alan R. Liss, Inc.; Culture of Animal Cells—A Manual of Basic Technique, Ian Freshney, N.Y., Alan R. Liss, Inc., 1987), and U.S. Pat. Nos. 6,670,180; 6,048,728; 6,692,961; and 6,103,529, all of which are incorporated by reference herein.

If it is desired to produce a culture medium containing serum, a basal medium containing serum may be used, or serum may be added, or a medium may be made from scratch to include serum. One exemplary serum type commonly used in the art is fetal or new-born calf serum. Typically, serum contains substances that inhibit collagenases; thus, collagenase concentrations may need to be adjusted when serum is used compared to when the medium is serum-free. An exemplary inhibitor of collagenase found in serum, which may also be added even in some types of serum-free media, is bovine serum albumin.

b. MMP-1 and cAMP-Elevating Agents

In embodiments where the collagenase is MMP-1, the MMP-1 is generally present in the medium according to the invention at a concentration sufficient to support the growth and/or viability of the cells, and to enhance the culture through, e.g., increasing the proportion of the cells in a primary culture that are the desired cell type, increasing population doublings, delaying senescence, or a combination of these. The exact concentration may vary depending on the cell type in use and the other media components present, but may be easily determined using preliminary small scale tests in accordance with conventional practice. Thus, for example, for any chosen medium, cells may be cultured on a small scale in the presence of a range of MMP-1 concentrations and the optimum concentration determined by observing the effect of different concentrations on cell growth and viability. Similar routine experiments may be conducted to determine the optimal concentration of the cAMP-elevating agent, if used. Additionally, it may be useful to conduct experiments using a range of combinations of MMP-1 and cAMP-elevating agent if the two are to be used together in the culture medium.

In some embodiments the collagenase is present in the medium at concentrations measured in units/ml (U/ml), wherein the activity is due to collagenase not produced by cells in the medium. The collagenase may be present at greater than about 0.000001, 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, or 3.0 U/mil. The collagenase may be present at less than about 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, 3.0, 5.0, or 10.0 U/ml. E.g., the collagenase may be present at about 0.00001-0.1, 0.00005-0.5, 0.0001-0.1, or 0.0001-0.05 U/ml.

In some embodiments utilizing highly pure MMP-1, e.g., rhMMP-1, MMP-1 is present in the culture medium at a concentration from about 0.01 ug/ml to about 100 ug/ml, or about 0.05 ug/ml to about 50 ug/ml, or about 0.1 ug/ml to about 20 ug/ml, or about 0.1 ug/ml to about 10 ug/ml, or about 0.5 ug/ml to about 5.0 ug/ml, or about 0.8-3 ug/ml, or about 1.5-2.0 ug/ml, or about 1-3 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml. In some embodiments less highly purified MMP-1 may be used, e.g., crude MMP-1 from Clostridium histolyticum which, in some embodiments, has been partially purified, e.g., to remove endotoxins. Such MMP-1 preparations may be present at a concentration of about 0.02 ug/ml to about 200 ug/ml, or about 0.1 ug/ml to about 100 ug/ml, or about 0.2 ug/ml to about 40 ug/ml, or about 0.2 ug/ml to about 20 ug/ml, or about 1.0 ug/ml to about 10 ug/ml, or about 1-7 ug/ml, or about 2-3.5 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml.

In some embodiments, MMP-1 and one or more cAMP-elevating agents are present in the cell culture medium. The cAMP-elevating agent may be present in the culture medium at a concentration from about 0.01 ug/ml to about 1000 ug/ml, or about 0.01 ug/ml to about 100 ug/ml, or about 0.05 ug/ml to about 50 ug/ml, or about 0.1 ug/ml to about 20 ug/ml, or about 0.1 ug/ml to about 10 ug/ml, or about 0.5 ug/ml to about 5.0 ug/ml, or about 1.0 ug/ml to about 5 ug/ml, or about 0.1, 0.2, 0.3, 0.4, 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml.

In some embodiments of cell culture medium, the cAMP-elevating agent is forskolin present at a concentration of about 0.02-200 ug/ml, or about 0.1 and 20 ug/ml, or about 0.1-10 ug/ml, or about 0.1-5 ug/ml, or about 0.2-20 ug/ml, or about 0.4-10 ug/ml, or about 0.8-5 ug/ml, or about 1.5-2.0 ug/ml, or about 1.0, 1.1, 1.2, 1.3, 1.4, 1.5, 1.6, 1.7, 1.8, 1.9, 2.0, 2.1, 2.2, 2.3, 2.4, or 2.5 ug/ml. In some embodiments of cell culture medium, the cAMP-elevating agent is cholera toxin present at a concentration of about 0.001-10 ug/ml, or about 0.01-1 ug/ml, or about 0.05-0.5 ug/ml, or about 0.08-0.1 ug/ml, or about 0.02, 0.03, 0.04, 0.05, 0.06, 0.07, 0.08, 0.09, 0.10, 0.11, 0.12, 0.13, or 0.14 ug/ml. In some embodiments of cell culture medium, the cAMP-elevating agent is dibutyryl cAMP present at a concentration of about 1.5-15,000 ug/ml, or about 15-1500 ug/ml, or about 30-750 ug/ml, or about 60-300 ug/ml, or about 100-200 ug/ml or about 100, 110, 120, 130, 140, 150, 160, 170, 180, 190, or 200 ug/ml. In some embodiments of cell culture medium, the cAMP-elevating agent is isobutylmethylxanthinge (IBMX) present at a concentration of about 0.07-700 ug/ml, or about 0.7-70 ug/ml, or about 1.5-35 ug/ml, or about 3.5-15 ug/ml, or about 5-10 ug/ml, or about 1, 2, 3, 4, 5, 6, 7, 8, 9, 10, 11, 12, 13, 14, or 15 ug/ml. In some embodiments of cell culture medium, the cAMP-elevating agent is theophylline present at a concentration of about 0.07-700 ug/ml, or about 0.7-70 ug/ml, or about 1.5-35 ug/ml, or about 3.5-15 ug/ml, or about 5-10 ug/ml, or about 1, 2, 3, 4, 5, 6, 7, 8, 9, 10, 11, 12, 13, 14, or 15 ug/ml. In some embodiments of cell culture medium, the cAMP-elevating agent is isoproterenol present at a concentration of about 0.003-30 ug/ml, or about 0.03-3 ug/ml, or about 0.06-1.5 ug/ml, or about 0.1-1 ug/ml, or about 0.05, 0.1, 0.15, 0.2, 0.25, 0.3, 0.35, 0.4, 0.45, 0.5, 0.55, or 0.6 ug/ml. In some embodiments of cell culture medium, the cAMP-elevating agent is PGE₂ present at a concentration of about 0.001-10 ug/ml, or about 0.01-1 ug/ml, or about 0.05-10 ug/ml, or about 0.001-10 ug/ml, or about 0.01-1 ug/ml, or about 0.05, 0.1, 0.15, 0.2, 0.25, 0.3, 0.35, 0.4, 0.45, 0.5, 0.55, or 0.6 ug/ml.

If the cell culture medium is to be a serum-containing cell culture medium, it may be desirable not to add a cAMP-elevating agent to the medium or to the supplement, as serum typically contains such agents, and it may not be necessary to add an exogenous cAMP-elevating agent. In media intended for the culture of fibroblasts it may not be desirable to add a cAMP-elevating agent, as the combination of MMP-1 and cAMP-elevating agent generally has the effect of inducing apoptosis in the fibroblasts. While this is desirable for the culture of non-fibroblast cell-types, it is undesirable when culturing fibroblasts themselves.

The cell culture medium may be used for the culture of any suitable cell type. In some embodiments, the cell culture medium or supplement containing MMP-1, in some embodiments also containing a cAMP-elevating agent, is intended for use with a cell type selected from the group consisting of fibroblasts, osteoblasts, chondrocytes, Schwann cells, neurons, hepatocytes, cardiomyocytes, and keratinocytes.

c. Additional Ingredients

The compositions of the invention may include additional ingredients useful in the culture of a particular cell type or of cells in general.

In cell culture medium that is serum-containing, serum can be added if not already present in the basal cell culture medium, or if the cell culture medium is produced from scratch. Serum contains a number of biochemical entities that the cells need for survival. Some of these entities protect the cells against toxic impurities, some of which may be products of the cultured cells themselves, and others serve to present iron and trace metals to the cells in a way the cells can use. The addition of serum can produce a well functioning medium for many different cell types. As described above, in cell culture media containing serum, it may not be necessary to add a cAMP-elevating agent, as serum typically contains such agents. The serum can be pathogen free and carefully screened for mycoplasma bacterial, fungal, and viral contamination. Also, the serum should generally be obtained from the United States and not obtained from countries where indigenous livestock carry transmittable agents. Serum for cell culture is generally fetal calf serum (FCS) or newborn calf serum. While FCS is the most commonly applied supplement in animal cell culture media, other serum sources are also routinely used, including newborn calf, horse and human.

In cell culture medium that is animal product-containing, extracts from organs or glands may also be used for the supplementation of culture media. These include extracts of submaxillary gland (see, e.g., Cohen, S., J. Biol. Chem. 237:1555-1565 (1961)), pituitary (see, e.g., Peehl, D. M., and Ham, R. G., In Vitro 16:516-525 (1980); U.S. Pat. No. 4,673,649), hypothalamus (see, e.g., Maciag, T., et al., Proc. Natl. Acad. Sci. USA 76:5674-5678 (1979); Gilchrest, B. A., et al., J. Cell. Physiol. 120:377-383 (1984)), ocular retina (see, e.g., Barretault, D., et al., Differentiation 18:29-42 (1981)) and brain (see, e.g., Maciag, T., et al., Science 211:1452-1454 (1981)). These types of chemically undefined supplements serve several useful functions in cell culture media. In some embodiments, the cell culture medium or supplement contains bovine pituitary extract (BPE).

A serum-free culture medium may contain BPE at an appropriate concentration for the growth of the cells for which it is intended. For animal protein-free, animal product-free, or defined media, BPE is not appropriate. Alternatively, an admixture of heparin, epidermal growth factor (EGF), a cAMP-increasing agent(s) and fibroblast growth factor(s) (FGF(s)) may be used as a replacement for BPE or other organ/gland extracts in animal cell culture media. If the source of the proteins is not of animal origin (e.g., if the proteins are recombinantly produced), then such an admixture may be appropriate for an animal product-free or animal protein-free medium. See below for a further description of these ingredients. See also U.S. Pat. No. 6,692,961, the disclosure of which is incorporated herein by reference.

Growth factors may be added to the cell culture medium or supplement. An example of a growth factor of use in the compositions of the invention is epidermal growth factor (EGF). EGF may be natural or recombinant and may be, e.g., human or rodent. EGF is available commercially (e.g., from GIBCO/LTI, Gaithersburg, Md.), or may be isolated from natural sources or produced by recombinant DNA techniques (U.S. Pat. No. 4,743,679) according to methodologies that are routine in the art. EGF can be added to the cell culture medium at a concentration of about 0.01-10,000 ng/ml, or about 0.1-0-100 ng/ml, or about 0.002-20 ng/ml, or about 0.02-2 ng/ml, or about 0.04-1 ng/ml, or about 0.08-0.8, or about 0.05, 0.1, 0.2, 0.3, 0.4, or 0.5 ng/ml. In some embodiments, EGF is present at about 0.2 ng/ml.

In addition, any of the fibroblast growth factor (FGF) family may be used, including FGF-1 (acidic FGF or aFGF), FGF-2 (basic FGF or bFGF), FGF-3 (int-2), FGF4 (K-FGF), FGF-5 (hst-1), FGF-6 (hst-2) and FGF-7 (keratinocyte growth factor or KGF). Natural or recombinant FGF may be used, which may be of human, bovine, porcine or rodent origin. For example, recombinant human aFGF may be used. aFGF, bFGF and KGF are available commercially (e.g., from GIBCO/LTI, Gaithersburg, Md. and R&D Systems, Inc., Minneapolis, Minn.), or may be isolated from natural sources or produced by recombinant DNA techniques (EP 0 408 146 and U.S. Pat. No. 5,395,756 for aFGF; U.S. Pat. No. 5,189,148 for bFGF; WO 90/08771 and WO 95/01434 for KGF) according to methodologies that are routine in the art. FGF can be added to the medium to a concentration of about 0.1-10,000 ng/ml, or about 1-100 ng/ml, or about 1-10 ng/ml, or about 1, 2, 3, 4, 5, 6, 7, 8, 9, or 10 ng/ml. In some embodiments FGF-1 is present at about 5 ng/ml. Other growth factors that may be added include HGF, Heregulin, NGF, or other growth factors depending on the cell type to be cultured.

Other ingredients useful in cell culture media or supplements of the invention include insulin, transferrin, hydrocortisone, and heparin. Insulin may be present at a concentration of about 0.05-500 ug/ml, or about 0.5-50 ug/ml, or about 1-25 ug/ml, or about 2-15 ug/ml, or about 1, 2, 3, 4, 5, 6, 7, 8, 9, or 10 ug/ml. In some embodiments, insulin is present at 5 ug/ml. Transferrin may be present at a concentration of about 0.1-10,000 ug/ml, or about 1-100 ug/ml, or about 2-20 ug/ml, or about 5-15 ug/ml, or about 5, 6, 7, 8, 9, 10, 11, 12, 13, 14, or 15 ug/ml. Hydrocortisone may be present at a concentration of about 0.001-10 ug/ml, or about 0.01-1 ug/ml, or about 0.02-0.5 ug/ml, or about 0.1-0.2 ug/ml. Heparin may be obtained commercially, for example from Sigma (Saint Louis, Mo.), and is derived, e.g., from porcine mucosa. Heparin is added to the present media primarily to stabilize the activity of the growth factor components, especially FGF. To formulate the medium of the present invention, heparin is added at a concentration of about 1-500 U.S.P. units/liter, or about 5-50 U.S.P. units/liter, or about 5-20 USP/L, or about 10-15 USP/L.

Ascorbic acid may also be added to the medium. Ascorbic acid is available commercially in several forms. An exemplary ascorbic acid for use in formulating the present media is L-ascorbic acid phosphate, magnesium salt, available from Wako Pure Chemical Industries. Ascorbic acid can be added to the medium at a concentration of about 0.001-10 mg/L, or about 0.01-5 mg/L. In some embodiments, ascorbic acid is present at a concentration of about 0.1 mg/L.

Further additional ingredients for culture media of the invention include purines, glutathione monobasic sodium phosphates, sugars, deoxyribose, ribose, nucleosides, lipids, acetate salts, phosphate salts, HEPES, phenol red, pyruvate salts and buffers. Other ingredients often used in media include steroids and their derivatives, cholesterol, fatty acids and lipids, Tween 80, 2-mercaptoethanol, pyramidines antibiotics (gentamicin, penicillin, streptomycin, amphotericin B, etc.) whole egg ultra filtrate, and attachment factors (fibronectins, vitronectins, collagens, laminins, tenascins, etc.). The concentrations of the ingredients are well known to one of ordinary skill in the art.

For cell culture media made from scratch, or for additional supplementation of conventional basal media, the ingredients are well-known in the art. Such ingredients are useful when the medium is, e.g., a defined medium. See, e.g., Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc.; Culture of Animal Cells—A Manual of Basic Technique, Ian Freshney, N.Y., Alan R. Liss, Inc., (1987); Culture of Epithelial Cells, Freshney, R. I., ed., New York: Wiley-Liss, (1992); Animal Cell Biotechnology, Vol. 1, Spier, R. E. et al., Eds., Academic Press New York (1985); Ham, R. G., Methods for Preparation of Media, Supplements and Substrata for Serum-Free Animal Culture, Alan R. Liss, Inc., New York (1984)) Waymouth, C., Methods for Preparation of Media, Supplements and Substrata for Serum-Free Animal Culture, Alan R. Liss, Inc., New York (1984); Cell & Tissue Culture: Laboratory Procedures, John Wiley & Sons Ltd., Chichester, England 1996; and U.S. Pat. Nos. 6,833,271; 6,692,961; 6,593,140; 6,103,529; 6,323,025; and 6,048,728, all of which are incorporated by reference herein in their entirety.

Briefly, for media made from scratch, or for supplementation of conventional basal media, ingredients may include amino acids, vitamins, inorganic salts, adenine, ethanolamine, D-glucose, heparin (mentioned above), N-[2-hydroxyethyl]piperazine-N′-[2-ethanesulfonic acid] (HEPES), hydrocortisone (mentioned above), insulin (mentioned above), lipoic acid, phenol red, phosphoethanolamine, putrescine, sodium pyruvate, triiodothyronine (T3), thymidine and transferrin (mentioned above). Alternatively, insulin and transferrin may be replaced by ferric citrate or ferrous sulfate chelates. Each of these ingredients may be obtained commercially, for example from Sigma (Saint Louis, Mo.).

Amino acid ingredients which may be included in the media of the present invention include L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. These amino acids may be obtained commercially, for example from Sigma (Saint Louis, Mo.). In some embodiments it may be useful to include the D-form of any of the amino acids listed above, with the exception of glycine.

Vitamin ingredients which may be included in the media of the present invention include biotin, choline chloride, D-Ca⁺⁺-pantothenate, folic acid, i-inositol, niacinamide, pyridoxine, riboflavin, thiamine and vitamin B₁₂. Formulations may also include fat soluble vitamins (including A, D, E and K). Vitamins may be obtained commercially, for example from Sigma (Saint Louis, Mo.).

Inorganic salt ingredients which may be used in the media of the present invention include a calcium salt (e.g., CaCl₂), CuSO₄, FeSO₄, KCl, a magnesium salt (e.g., MgCl₂), a manganese salt (e.g., MnCl₂), Sodium acetate, NaCl, NaHCO₃, Na₂HPO₄, Na₂SO₄ and ions of the trace elements selenium, silicon, molybdenum, vanadium, nickel, tin and zinc. These trace elements may be provided in a variety of forms, preferably in the form of salts such as Na₂SeO₃, Na₂SiO₃, (NH₄)₆Mo₇O₂₄, NH₄VO₃, NiSO₄, SnCl and ZnSO4. These inorganic salts and trace elements may be obtained commercially, for example from Sigma (Saint Louis, Mo.).

If the medium is made from scratch, the medium ingredients can be dissolved in a liquid carrier. The pH of the medium typically is adjusted to about 7.0-7.6, or about 7.1-7.5, or about 7.2-7.4. The osmolarity of the medium typically is adjusted to about 275-350 mOsm, or about 285-325 mOsm, or about 280-310 mOsm. The type of liquid carrier and the method used to dissolve the ingredients into solution vary and can be determined by one of ordinary skill in the art with no more than routine experimentation. Generally, the medium ingredients can be added in any order.

The culture media or additives of the present invention can be sterilized to prevent unwanted contamination. Sterilization may be accomplished, for example, by filtration through a low protein-binding membrane filter of about 0.1-1.0 um pore size (available commercially, for example, from Millipore, Bedford, Mass.) after admixing the concentrated ingredients, to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

d. Concentrated Solutions and Additives

In addition to providing culture media that are complete and ready-to-use, the invention also provides concentrated media, and additives for addition to conventional basal media to enhance cell growth, purity, and/or viability. For concentrated media, or for additives for addition to basal media, the solutions containing ingredients are more concentrated than the concentration of the same ingredients in a 1× media formulation. The ingredients can be 10-fold more concentrated (10× formulation), 25-fold more concentrated (25×formulation), 50-fold more concentrated (50× formulation), or 100-fold more concentrated (100× formulation). More highly concentrated formulations can be made, provided that the ingredients remain soluble and stable. See, e.g., U.S. Pat. No. 5,474,931, which is directed to methods of solubilizing culture media components at high concentrations. In some embodiments, various components of a medium are supplied at different concentration levels.

Media or supplements of the invention may also be provided as a lyophilized powder. As will be apparent, the proper amount of MMP-1 and/or other components to produce the proper final concentration when admixed with a predetermined volume of culture medium or other appropriate diluent may be supplied in appropriate packaging.

If the media or additives are prepared as separate concentrated solutions, or as lyophilized powders, an appropriate (sufficient) amount of each concentrate is combined with a diluent to produce a 1× medium formulation. The diluent used can water or other solutions including aqueous buffers, aqueous saline solution, or other aqueous solutions. In some embodiments the diluent is a convention basal or cell culture medium, to which an additive of the invention is added in concentrated or lyophilized form.

In some embodiments, the cell culture medium or supplement is packaged for transport, storage and/or use by a consumer. Such packaging of tissue culture medium for transport, storage, and/or use is well-known in the art. Packaged medium may include further components for the dispensing and storage of the medium, and may also include separately packaged diluent for dilution of concentrated medium, optional additional ingredients for inclusion by the user if desired, instructions for use, and the like.

e. Media for Culturing Keratinocytes.

An exemplary medium of the invention is a medium for culturing keratinocytes. Similar media, with appropriate adjustments, for the culutre of non-keratinocyte cells are also provided by the invention. To simplify description, the keratinocyte medium is described in detail below. It is to be understood that the description also applies to other media, e.g., media for epithelial cells and media for non-epithelial cells. In the former case, a cAMP-elevating agent is often used in the medium in addition to a collagenase. In the latter case, it is often not necessary to use a cAMP-elevating agent in addition to the collagenase. Also, as described herein, certain additions to the medium, e.g., serum, contain cAMP-elevating agents and additional agents may not be necessary if these are used. Other cell types for which media may be provided are as described herein. In some embodiments, the medium is for culture of a cell from brain, heart, lung, stomach, intestines, thyroid, adrenal, thymus, parathyroid, testes, liver, kidney, bladder, spleen, pancreas, gall bladder, ovaries, uterus, prostate, reproductive cells, lymph nodes, bone, cartilage, interstitial cells, blood cells, skin cells, or immunocytes. In some embodiments, the medium is formulated for culture of fibroblasts, osteoblasts, Schwann cells, neurons, cardiomyocytes, and hepatocytes. Although the basal medium used may be different for these cell types, and cAMP-elevating agents may not be necessary for non-epithelial cells, in general the media are composed as described below. Adjustments to the compositions are a matter of routine experimentation.

In some embodiments, the invention provides cell culture medium or supplement for culturing keratinocytes that contains an exogenous collagenase (i.e., a collagenase that is not produced by cells in the medium), e.g., exogenous MMP-1, and a cAMP-elevating agent.

In some embodiments of cell culture medium for keratinocytes, a basal commercial keratinocyte cell culture medium is used and additional ingredients are added to the basal medium. Exemplary basal media include, for example, MCDB 153, or GIBCO Keratinocyte Serum-Free Medium, or other media formulations readily apparent to those skilled in the art, including those described in U.S. Pat. No. 6,692,961, and in Methods For Preparation of Media, Supplements and Substrate For Serum-Free Animal Cell Culture Alan R. Liss, New York (1984) and Cell & Tissue Culture: Laboratory Procedures, John Wiley & Sons Ltd., Chichester, England 1996, all of which are incorporated by reference herein in their entirety. It will be appreciated that a keratinocyte culture medium containing a collagenase, e.g., MMP-1, and a cAMP-elevating agent can also be prepared “from scratch” using art-accepted techniques, as described herein.

The medium or supplement may be prepared as serum-containing, serum-free, animal protein-free, animal product-free, or defined, as desired or necessary for the intended use. Methods for preparing such media are known in the art. Particularly useful are serum-free and animal-product-free culture media for keratinocytes.

Keratinocyte culture media of the invention contain exogenous collagenase (i.e., collagenase that is not produced by cells in the medium). In some embodiments, the medium contains exogenous MMP-1. The exogenous collagenase, e.g., MMP-1 of the keratinocyte culture medium may be any of the types described herein. In some embodiments of cell culture medium for the culture of keratinocytes, less highly purified collagenase, e.g., MMP-1 may be used, e.g., crude collagenase, e.g., MMP-1 from bacteria, e.g., collagenase preparation from Clostridium histolyticum. In some embodiments, the collagenase preparation has been partially purified, e.g., to remove endotoxins. In some embodiments, the collagenase is highly purified. In some embodiments, the collagenase is low-endotoxin. Such collagenase (MMP-1) preparations may be present at a concentration of about 0.02 ug/ml to about 200 ug/ml, or about 0.1 ug/ml to about 100 ug/ml, or about 0.2 ug/ml to about 40 ug/ml, or about 0.2 ug/ml to about 20 ug/ml, or about 10 ug/ml to about 10 ug/ml, or about 1-7 ug/ml, or about 2-3.5 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml. In some embodiments the collagenase is present in the medium at concentrations measured in units/ml (U/ml), wherein the activity is due to collagenase not produced by cells in the medium. The collagenase may be present at greater than about 0.000001, 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, or 3.0 U/ml. The collagenase may be present at less than about 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, 3.0, 5.0, or 10.0 U/ml. E.g., the collagenase may be present at about 0.00001-0.1, 0.00005-0.5, 0.0001-0.1, or 0.0001-0.05 U/ml.

In some embodiments utilizing highly pure collagenase, e.g., a highly pure MMP-1, e.g., rhMMP-1, is present in the culture medium at a concentration from about 0.01 ug/ml to about 100 ug/ml, or about 0.05 ug/ml to about 50 ug/ml, or about 0.1 ug/ml to about 20 ug/ml, or about 0.1 ug/ml to about 10 ug/ml, or about 0.5 ug/ml to about 5.0 ug/ml, or about 0.8-3 ug/ml, or about 1.5-2.0 ug/ml, or about 1-3 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml. In some embodiments less highly purified MMP-1 may be used, e.g., crude MMP-1 from Clostridium histolyticum which, in some embodiments, has been partially purified, e.g., to remove endotoxins. Such MMP-1 preparations may be present at a concentration of about 0.02 ug/ml to about 200 ug/ml, or about 0.1 ug/ml to about 100 ug/ml, or about 0.2 ug/ml to about 40 ug/ml, or about 0.2 ug/ml to about 20 ug/ml, or about 1.0 ug/ml to about 10 ug/ml, or about 1-7 ug/ml, or about 2-3.5 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml.

In some embodiments, the medium further contains a cAMP-elevating agent. The cAMP-elevating agent may be added alone or as part of a added serum, or a combination thereof. In some embodiments, the keratinocyte cell culture medium contains one or more of the following cAMP-elevating agents: forskolin at a concentration of about 0.5-5 ug/ml, e.g., about 1.5-2 ug/ml; cholera toxin at a concentration of about 0.01 to 0.5 ug/ml, e.g., about 0.08-0.1 ug/ml; dibutyryl cAMP at a concentration of about 50-300 ug/ml, e.g., about 150 ug/ml; IBMX at a concentration of about 2-12 ug/ml, e.g., about 7 ug/ml; theophylline at a concentration of about 2-12 ug/ml, e.g., about 7 ug/ml; isoproterenol at a concentration of about 0.1-0.5 ug/ml, e.g., about 0.3 ug/ml; and/or PGE2 at a concentration of about 0.02 to 1 uM, e.g., about 0.1-0.2 uM. The forgoing concentrations are for each agent used individually; it will be appreciated that if more than one agent is used in combination, the concentrations of one or more of the agents may be adjusted downward.

In some embodiments, the keratinocyte culture media of the invention include a collagenase and forskolin. The collagenase may be any of the collagenases described herein, e.g., bacterial collagenase, MMP, such as MMP-1, e.g., recombinant human MMP-1. In some embodiments, the keratinocyte culture media of the invention include rh MMP-1 at a concentration of about 0.1-5 ug/ml, or about 0.5-5 ug/ml, or about 1-3 ug/ml, or about 1.5-2 ug/ml, or about 1.5, 1.6, 1.7, 1.8, 1.9, or 2.0 ug/ml and further include forskolin at a concentration of about 0.02-200 ug/ml, or about 0.1 and 20 ug/ml, or about 0.1-10 ug/ml, or about 0.1-5 ug/ml, or about 0.2-20 ug/ml, or about 0.4-10 ug/ml, or about 0.8-5 ug/ml, or about 1.5-2.0 ug/ml, or about 1.0, 1.1, 1.2, 1.3, 1.4, 1.5, 1.6, 1.7, 1.8, 1.9, 2.0, 2.1, 2.2, 2.3, 2.4, or 2.5 ug/ml, e.g., about 1.5-2 ug/ml. In some embodiments, the keratinocyte culture media of the invention include MMP-1 from bacteria and forskolin. In some embodiments, the keratinocyte culture media of the invention contain MMP-1 from bacteria at a concentration of about 0.02 ug/ml to about 200 ug/ml, or about 0.1 ug/ml to about 100 ug/ml, or about 0.2 ug/ml to about 40 ug/ml, or about 0.2 ug/ml to about 20 ug/ml, or about 1.0 ug/ml to about 10 ug/ml, or about 1-7 ug/ml, or about 2-3.5 ug/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ug/ml, or at concentrations measured in units/ml (U/ml), wherein the activity is due to collagenase not produced by cells in the medium, such as at greater than about 0.000001, 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, or 3.0 U/ml, less than about 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, 3.0, 5.0, or 10.0 U/ml, e.g., the collagenase may be present at about 0.00001-0.1, 0.00005-0.5, 0.0001-0.1, or 0.0001-0.05 U/ml; and further includes forskolin at a concentration of about 0.02-200 ug/ml, or about 0.1 and 20 ug/ml, or about 0.1-10 ug/ml, or about 0.1-5 ug/ml, or about 0.2-20 ug/ml, or about 0.4-10 ug/ml, or about 0.8-5 ug/ml, or about 1.5-2.0 ug/ml, or about 1.0, 1.1, 1.2, 1.3, 1.4, 1.5, 1.6, 1.7, 1.8, 1.9, 2.0, 2.1, 2.2, 2.3, 2.4, or 2.5 ug/ml, e.g., about 1.5-2 ug/ml. e.g., about 1.5-2 ug/ml.

In some embodiments, the keratinocyte culture medium of the invention may contain serum. In such embodiments, it may not be necessary to add a further exogenous cAMP-elevating agent, as serum itself generally contains such agents.

In some embodiments the keratinocyte culture medium of the invention further contains, in addition to MMP-1 and a cAMP-elevating agent, one or more of the following: insulin at a concentration of about 1-10 ug/ml, e.g., about 5 ug/ml; transferrin at a concentration of about 5-20 ug/ml, e.g., about 10 ug/ml; hydrocortisone at a concentration of about 0.05-0.1 ug/ml, e.g., about 0.1-0.2 ug/ml; EGF at a concentration of about 0.05-1 ng/ml, e.g., about 0.2 ng/ml; FGF-1 at a concentration of about 1-10 ng/ml, e.g., about 5 ng/ml; and heparin at a concentration of about 1-50 USP/L, e.g., about 10-15 USP/L.

In some embodiments, keratinocyte culture medium of the invention includes collagenase from Clostridium histolyticum, e.g., low-endotoxin collagenase and/or highly purified collagenase, at about 2-3.5 ug/ml, insulin at a concentration of about 5 μg/ml, transferrin at about 10 μg/ml, hydrocortisone at about 0.1-0.2 μg/ml, EGF at about 0.2 ng/ml, FGF-1 at about 5 ng/ml, heparin at about 10-15 USP/L, and forskolin at about 1.5-2.5 ug/ml. In some embodiments the medium further contains BPE at a concentration of 10-15 ug/ml. In some embodiments, keratinocyte culture medium of the invention includes rhMMP-1 at about 1.5-2.0 μg/ml, insulin at a concentration of about 5 μg/ml, transferrin at about 10 μg/ml, hydrocortisone at about 0.1-0.2 μg/ml, EGF at about 0.2 ng/ml, FGF-1 at about 5 ng/ml, heparin at about 10-15 USP/L, and forskolin at about 1.5-2.5 ug/ml. In some embodiments the medium further contains BPE at a concentration of about 10-15 ug/ml.

Keratinocyte culture media or additives of the invention may also include BPE. If BPE is added, it may be used at any concentration appropriate to the culture of keratinocytes. Exemplary concentrations are about 1 to about 100 ug/ml, or about 5 to about 75 ug/ml, or about 10 to about 50 ug/ml, or about 10, 15, 20, 25, 30, 35, 40, 45, or 50 ug/ml. In some embodiments, BPE is used at about 10-15 ug/ml. In some embodiments, BPE is used at about 30 ug/ml.

The invention also provides additives that contain exogenous MMP-1, for addition to a keratinocyte culture medium. In addition, the additives may contain a c-AMP-elevating agent, as well as any or all of the additional ingredients listed for keratinocyte culture medium. The additive is concentrated or lyophilized, and may be prepared in one or more parts, as described elsewhere herein.

The keratinocyte culture medium or additive may be packaged as described herein.

Other illustrative cell culture media include media for the culture of chondrocytes (see, e.g., Example 8), hepatocytes, (see, e.g., Example 10) and fibroblasts (see, e.g., Examples 4 and 9).

2. Methods

In one aspect, the invention provides methods of culturing cells. Cells are cultured in media that contain exogenous collagenase, e.g., exogenous MMP-1. The media may also contain an exogenous cAMP-elevating agent. Other ingredients, as described for the production of cell culture media, may also be included.

a. Cell Types that May be Cultured

The compositions and methods of the invention are suitable for the culture of a variety of cells, especially eukaryotic cells. The media of the invention are suitable for culturing animal cells, especially mammalian cells; plant cells; insect cells; arachnid cells; and microorganisms such as bacteria, fungi, molds, protozoa, and rickettsia, including antibiotic-producing cells. Exemplary applications include the culture of cloned cells, such as hybridoma cell lines; of mammalian cells for the production of cell products, especially proteins and peptides such as hormones, enzymes, and immunofactors; of virally-infected cells for the production of vaccines; of plant cells in, for example, meristem or callus culture; of epithelial cells to provide tissue for wound healing; of resistant cells for medical and diagnostic use; and in media adapted for the production and preservation of biological organs and implant tissue.

Specific cell types useful for culture in the processes of the invention accordingly include: cells derived from mammalian tissues, organs and glands such as the brain, heart, lung, stomach, intestines, thyroid, adrenal, thymus, parathyroid, testes, liver, kidney, bladder, spleen, pancreas, gall bladder, ovaries, uterus, prostate, and skin; reproductive cells (sperm and ova); lymph nodes, bone, cartilage, and interstitial cells; blood cells including immunocytes, cytophages such as macrophages, lymphocytes, leukocytes, erythrocytes, and platelets. Additional cell types include stem, leaf, pollen, and ovarian cells of plants; microorganisms and viruses as specified above; and cells derived from insect or arachnid tissues, organs, and glands.

Mammalian and other cells particularly suitable for cultivation in the present media include those of human origin, which may be primary cells derived from a tissue sample, e.g. Stem cells (Human and mouse; Adult and Embryonic), Chicken Egg Fibroblasts, Microglia, Human and monkey skeletal muscle cells, Mast cells, Macrophages Eosinophils Human endothelial cells, Shwann cells Hippocampal neurons Astrocytes Monocytes Dorsal root ganglion, Neurons, Adipocytes, Kidney cells, Melanoma cells, Embryonic fibroblasts, Pancreatic beta cells, Beta islet, Embryonic cardiomyocytes, Intestinal epithelial cells, Hepatocytes, Bone marrow, T cells, Human Corneal Epithelial Model, Blood Brain Barrier, Bladder Cell, Endothelial Cell, Melanocyte Cell, Mammary Epithelial Cell, Smooth Muscle Cell, Skeletal Muscle Cell, Neural Cell, Prostate Cell, Renal Cell, Renal Epithelial Cell, Skeletal Cell, Lymphocytes, Monocytes, Bone Marrow, Peripheral Blood, Glial, Embryonic Stem Cell Mice, hematopoietic cells, Monkey Kidney Cells, Synovial Cells, and HUVEC. Further examples of mammalian cells include diploid cell strains such as MRC-5 and WI-38; transformed cells or established cell lines (e.g., HeLa), each of which may optionally be diseased or genetically altered. Other mammalian cells, such as hybridomas, CHO cells, COS cells (e.g., COS-7L), VERO cells (monkey kidney epithelial cells), HeLa cells, 293 cells (embryonal human kidney), reabbit kidney cells, PER-C6 cells, K562 cells, MOLT-4 cells, M1 cells, NS-1 cells, COS-7 cells, MDBK cells, MDCK cells, MRC-5 cells, WI-38 cells, WEHI cells, SP2/0 cells, BHK cells (including BHK-21 cells) and derivatives thereof, are also suitable for cultivation in the present media. In particular, stem cells and cells used in vitro virus production may be cultivated in the media of the present invention. Tissues, organs, organ systems and organisms derived from animals or constructed in vitro or in vivo using methods routine in the art may similarly be cultivated in the culture media of the present invention.

Primary culture of cell types of the invention includes, in some embodiments, culture of cells such as fibroblasts, osteoblasts, chondrocytes, Schwann cells, neurons, cardiomyocytes, hepatocytes, and keratinocytes.

The compositions and methods of the invention are particularly useful for culturing epithelial cells, e.g., keratinocytes. Successful culture of keratinocytes has proven to be difficult, owing primarily to their nutritional fastidiousness. Keratinocytes from skin are often rapidly overgrown by less fastidious and faster-growing fibroblasts that were also resident in the tissue. This is especially true in the culture of fetal keratinocytes, because, unlike in adult or neonatal skin, it is generally not possible to separate dermis from epidermis in fetal skin, and thus the dermal fibroblasts are present along with the epidermal keratinocytes in the initial culture.

As an example of cell culture, the culture of keratinocytes using media of the invention is described in more detail below. However, it is understood that the methods of the invention are applicable to a wide range of cell types and culture conditions, of which the culture of keratinocytes serves as an illustration and example.

b. Methods of Cell Culture

Animal cells for culturing by the present invention may be obtained commercially, for example from ATCC (Rockville, Md.), Cell Systems, Inc. (Kirkland, Wash.) or Invitrogen Corporation (San Diego, Calif.). Alternatively, cells may be isolated directly from samples of animal tissue obtained via biopsy, autopsy, donation or other surgical or medical procedure.

Generally, tissue should be handled using standard sterile technique and a laminar flow safety cabinet. In the use and processing of all human tissue, the recommendations of the U.S. Department of Health and Human Services/Centers for Disease Control and Prevention typically should be followed (Biosafety in Microbiological and Biomedical Laboratories, Richmond, J. Y. et al., Eds., U.S. Government Printing Office, Washington, D.C. 3rd Edition (1993)). The tissue is cut into small pieces (e.g., 0.5×0.5 cm) using sterile surgical instruments. The small pieces are washed twice with sterile saline solution supplemented with antibiotics, and then may be optionally treated with an enzymatic solution (e.g., collagenase or trypsin solutions, each available commercially, for example, from Life Technologies, Inc., Rockville, Md.) to promote dissociation of cells from the tissue matrix.

Cells may be isolated by any technique known or developed in the art. In one typical technique, the mixture of dissociated cells and matrix molecules are washed twice with a suitable physiological saline or tissue culture medium (e.g., Dulbecco's Phosphate Buffered Saline without calcium and magnesium). Between washes, the cells are centrifuged (e.g., at 200×g) and then resuspended in serum-free tissue culture medium. Aliquots may be counted using an electronic cell counter (such as a Coulter Counter). Alternatively, the cells can be counted manually using a hemocytometer.

The isolated cells can be plated according to the experimental conditions determined by the investigator. The examples below demonstrate at least one functional set of culture conditions useful for cultivation of certain mammalian cells. It is to be understood, however, that the optimal plating and culture conditions for a given animal cell type can be determined by one of ordinary skill in the art using only routine experimentation. For routine culture conditions, using the present invention, cells can be plated onto the surface of culture vessels without attachment factors. Alternatively, the vessels can be precoated with natural, recombinant or synthetic attachment factors or peptide fragments (e.g., collagen or fibronectin, or natural or synthetic fragments thereof).

Isolated cells can also be seeded into or onto a natural or synthetic three-dimensional support matrix such as a preformed collagen gel or a synthetic biopolymeric material, or onto feeder layers of cells. Use of attachment factors or a support matrix with the medium of the present invention will enhance cultivation of many attachment-dependent cells in the absence of serum supplementation. Thus, culture techniques useful in the methods of the invention include the use of solid supports, (especially for anchorage-dependent cells in, for example, monolayer or suspension culture) such as glass, carbon, cellulose, hollow fiber membranes, suspendable particulate membranes, and solid substrate forms, such as agarose gels. In the latter embodiments, it is possible to entrap the collagenase, e.g., MMP-1, e.g., it can be caged within the bead, trapped within the matrix, or covalently attached, i.e. as a mixed disulfide.

The cell seeding densities for each experimental condition can be optimized for the specific culture conditions being used. For routine culture in plastic culture vessels, an initial seeding density of 0.1-1.0×10⁵ cells per cm² or about 1.5× the plating concentration routinely used for the same cells in serum supplemented media is preferable.

Mammalian cells are typically cultivated in a cell incubator at about 37° C., while the optimal temperatures for cultivation of avian, nematode and insect cells are typically somewhat lower and are well-known to those of ordinary skill in the art. The incubator atmosphere should be humidified for cultivation of animal cells, and should contain about 3-10% carbon dioxide in air. Culture medium pH should be in the range of about 7.1-7.6, in some embodiments about 7.1-7.4, and in some embodiments about 7.1-7.3.

Cells in closed or batch culture should undergo complete medium exchange (i.e., replacing spent media with fresh media) about every 2-3 days, or more or less frequently as required by the specific cell type. Cells in perfusion culture (e.g., in bioreactors or fermenters) will receive fresh media on a continuously recirculating basis.

The methods of the invention include culturing cells in a medium that contains exogenous collagenase, e.g., exogenous MMP-1. In some embodiments, the methods of the invention include culturing cells in a medium that contains exogenous collagenase, e.g., exogenous MMP-1, and a cAMP-elevating agent. The methods of the invention are useful in primary cultures; serial cultures; subcultures; preservation of cultures, such as frozen or dried cultures; and encapsulated cells; cultures also may be transferred from conventional media to media containing MMP-1 and, optionally, a cAMP-elevating agent, by known transfer techniques.

According to the practice of the invention, cells are exposed to exogenous collagenase, e.g., MMP-1, and, in some embodiments, a cAMP-elevating agent, in amounts sufficient to promote culture of these cells in vitro, as measured, for example, by significant increase in purity of the cell culture for the cell type of interest, increase in cell lifespan, increase in cell viability, increase in cell biomass, increase in cell bioproductivity, delay of cell senescence, or diversification or normalization of cell function as compared to unexposed cells.

In certain embodiments of the subject method, it will be desirable to monitor the growth state of cells in the culture, e.g., cell proliferation, differentiation and/or cell death. Methods of measuring cell proliferation are well known in the art and most commonly include determining DNA synthesis characteristic of cell replication. There are numerous methods in the art for measuring DNA synthesis, any of which may be used according to the invention. For example, DNA synthesis can be determined using a radioactive label (³H-thymidine) or labeled nucleotide analogues (BrdU) for detection by immunofluorescence. Cell nuclei that have incorporated BrdU during DNA synthesis can be identified using mouse monoclonal anti-BrdU (Dako; Carpintaria, Calif.), detected with the immuno-peroxide technique of Sternberger et al., J. Histochem., Cytochem. 18: 315 (1970), followed by hematoxylin counterstaining.

c. Methods of Cell Culture to Produce an Animal Cell Product.

The invention also provides methods of cell culture to produce an animal cell product, where the cell culture is exposed at some point during culture of the cells to exogenous collagenase, e.g., MMP-1, and, in some embodiments, to a cAMP-elevating agent. Thus, media according to the invention may be used to culture animal cells to obtain an animal cell product. In some embodiments, the invention provides a process for obtaining an animal cell product by cell culture which comprises the steps of (1) culturing animal cells which produce said product in a nutrient culture medium comprising assimilable sources of carbon, nitrogen, amino acids, iron and other inorganic ions, trace elements and optionally lipids and growth promoters or regulators in admixture with a collagenase, e.g., MMP-1, and, in some embodiments, one or more cAMP-elevating agents, (2) continuing the culture until said product accumulates and (3) recovering said product.

Cell products which may be obtained according to the invention include any products that are produced by cultured animal cells. Typical products include polypeptides and proteins, for example immunoglobulins such as monoclonal and recombinant antibodies and fragments thereof, hormones such as erythropoietin and growth hormone, e.g. human growth hormone, lymphokines such as interferon, interleukins such as interleukin 2, 4, 5 and 6 and industrially and therapeutically useful enzymes such as tissue plasminogen activator.

In the process according to the invention, the animal cells may generally be cultured in suspension in the culture medium in a suitable culture vessel, for example a stirred tank or airlift fermenter, using known culture techniques. The production of the desired products during the culture may be monitored using any appropriate assay for the particular product in question. Thus, for example, where the product is a polypeptide or protein, the production of this may be monitored by general assay techniques such as enzyme-linked immunoabsorbent assay or immunoradiometric assay adapted for use with the particular polypeptide or protein.

Where in the process according to the invention it is desired to isolate the cell product obtained, this may be achieved using conventional separation and purification techniques. Thus, for example, where the product is secreted by the cells into the medium it may be separated from the cells using techniques such as centrifugation and filtration and then further purified using, for example, affinity purification techniques, such as affinity chromatography. Where the product is not secreted by the cells, the above methods may still be used, but after the cells have first been lysed to release the product

D. Keratinocyte Culture

1. General

In certain embodiments the present invention provides methods useful in keratinocyte culture systems where the cells are exposed to exogenous collagenase, e.g., MMP-1, and a cAMP-elevating agent. In some embodiments, the methods of the invention are used in the culture of keratinocytes in serum-free culture systems or animal product-free culture systems. The methods of the invention are particularly useful for cultivating keratinocytes, especially fetal keratinocytes, because these cells tend to be displaced in culture by fibroblasts, whose growth and survival is discouraged by the presence of a collagenase, e.g., MMP-1, and a cAMP-elevating agent. Thus, the methods of the invention can be used to provide extremely pure and long-lived cultures of keratinocytes.

2. Methods

Any source of keratinocytes may be used in the methods of the invention. The keratinocytes may be of animal or human origin, and may be from fetal, newborn, juvenile, or adult organisms.

Typically, the initial source of keratinocytes is skin. Skin can be obtained by appropriate biopsy or upon autopsy. In the case of animal skin, the animal may be sacrificed and skin removed and treated after sacrifice. For newborn keratinocytes, especially human keratinocytes, a common source is neonatal foreskin. In some embodiments, the tissue (skin) is cleaned, removed and placed in appropriate medium, e.g., Dulbecco's Modified Eagle's Medium (DMEM). Other media may be used, as will be apparent to those of skill in the art. If the skin is fetal, in some embodiments the source may be mouse or rat, but any source of fetal skin may be used. A common source of non-fetal, e.g., neonatal skin, is from newborn humans (e.g., foreskin).

Skin from newborn or adult animals may be treated mechanically to remove epidermis from dermis by techniques well-known in the art. Skin from fetal animals generally cannot be separated into dermis and epidermis by mechanical means, and can be treated by digestive enzyme(s), e.g., protease instead. Thus, either mechanically separated epidermis, or whole skin (e.g., fetal), may be treated with protease. For example, the skin or epidermis can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mechanically disrupting the tissue and treating the disrupted tissue with any of a number of digestive enzymes either alone or in combination. These include but are not limited to trypsin, chymotrypsin, collagenase, elastase, and/or hyaluronidase, DNase, pronase, dispase, and the like. Mechanical disruption can be accomplished by a number of methods including, but not limited to, the use of grinders, blenders, sieves, homogenizers, pressure cells, or insonators to name but a few. For a review of tissue disaggregation techniques, see Freshney, Culture of Animal Cells: A Manual of Basic Technique, 2d Ed., A. R. Liss, Inc., New York, 1987, Ch. 9, pp. 107-126.

In one embodiment, the skin is chopped with scissors, then incubated in 0.3% Dispase & trypsin 0.2% (1/1 dilution). Incubation may be at any temperature where the protease is active; e.g., between about 5° C. and about 60° C., or about 10° C. and about 50° C., or about 20° C. and about 45° C., or about 25° C. and about 40° C., or about 30° C. and about 40° C. In some embodiments, the skin and protease are incubated at about 32, 33, 34, 35, 36, 37, 38, 39, or 40° C. In some embodiments, a temperature of about 37° C. is used. The skin/protease mixture is incubated for a length of time that allows separation of cells without significant damage to the desired cells, i.e., keratinocytes. The incubation time may be, e.g., between about 0.25 hour and about 20 hours, or about 0.5 hour and about 15 hours, or about 0.5 hour and about 10 hours, or about 0.5 hours and about 5 hours. In some embodiments, the incubation time is about 1 to about 3 hours, or about 1 to about 2 hours. In some embodiments, the incubation time is less than about 5, 4, 3, 2 or 1 hours. In some embodiments, the incubations time is about 1 hour. In some embodiments, the incubation time is about 1.5 hours. In some embodiments, the incubation time is about 2 hours. The incubation medium may be mixed, e.g., every 15-20 minutes. Surprisingly, it has been found that incubation times of about 1 to about 2 hours produce the best results for fetal skin, in contrast to longer incubation times (typically overnight) that are used in common protocols for obtaining keratinocytes

After incubation the cells are dissociated, e.g., with a 10 ml glass pipet. The dissociated cells are passed through a cell strainer, e.g., a 75 μm strainer. The cells are washed in medium (e.g., DMEM) and centrifuged. Exemplary washing and centrifugation protocols include centrifugation at about 800 rpm for 5-10 minutes, with the wash and centrifugation steps repeated at least three times.

At this point the cells are ready for culture. Optionally, the cells may be further treated to enrich the starting culture in keratinocytes by methods well-known in the art; e.g., using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting. For a review of clonal selection and cell separation techniques, see Freshney, Culture of Animal Cells: A Manual of Basic Techniques, 2d Ed., A. R. Liss, Inc., New York, 1987, Ch. 11 and 12, pp. 137-168. However, a major advantage of the methods of the present invention is that cultures become enriched in keratinocytes without the necessity for such pretreatment of the cell population to be cultured; thus, such pretreatment is optional.

Cells may be cultured immediately after preparation, or may be stored. For storage, any suitable protocol, as known in the art, may be used.

Cells obtained as described are then cultured. The cells may be cultured in any manner known in the art including in monolayer, beads or in three-dimensions and by any means (i.e., culture dish, roller bottle, a continuous flow system, etc.). Methods of cell and tissue culturing are well known in the art, and are described, for example, in Cell & Tissue Culture: Laboratory Procedures, John Wiley & Sons Ltd., Chichester, England 1996; Freshney, Culture of Animal Cells: A Manual of Basic Technique, 2d Ed., A. R. Liss, Inc., New York, 1987, both of which are incorporated herein by reference in their entirety.

The cell culture medium may be any keratinocyte culture medium described herein. In addition, the initial culture of the cells may be in conventional medium, without any exogenous MMP-1 or cAMP-elevating agent; however, it is preferable to begin culturing the cells in medium of the invention that includes an exogenous MMP-1 and, generally, a cAMP-elevating agent, as described herein. In most embodiments of the invention, the culture medium is serum-free. In some embodiments of the invention, the medium is serum-containing. In some embodiments, the medium is animal product-free. In some embodiments, the medium is animal protein-free. The medium may be used with or without bovine pituitary extract (BPE). Although culture is improved with BPE, BPE adds unknown factors from a bovine source. It is not necessary for keratinocyte culture according to the methods of the invention and its use is optional.

The medium is replaced at intervals. The intervals may be regular or irregular. Replacement intervals can be from about 0.25 to about 4 days, or about 0.5 to about 2 days, or about 1 day. In some embodiments, the medium is replaced daily

The media may be replaced for about 1, 2, 3, 4, 5, 6, 7, 8, 9, 10, or more than about 10 days. In some embodiments, the media is replaced for about 4, about 5, or about 6 days. In some embodiments, the media is replaced for about 5 days.

After about the first 24-48 hours of cell culture, fibroblasts float to the top of the culture medium and can be removed by washing the keratinocytes (which adhere to the culture apparatus, e.g., plate). The fibroblasts may be discarded or they may be cultured separately. After about two days, the culture is >99% keratinocytes, because of the apoptosis and removal of fibroblasts. When using conventional methods of keratinocyte culture, even if just the epidermis is taken, significant fibroblast contamination can be seen in the culture. The degree of contamination can be quantitated by observing gene expression for genes expressed mainly by fibroblasts, and/or by examining the ratio of a gene expressed at high levels in fibroblasts gene to a gene expressed at low levels by keratinocytes. The latter method is useful when keratinocyte concentrations in the culture have become very high.

Thus, in some embodiments, the methods of the invention comprise the production of cultures of keratinocytes that comprise at least about 60, 70, 80, 90, 95, 98, 99, 99.5, 99.9, or 99.99% keratinocytes by culturing the keratinocytes in the presence of exogenous MMP-1 and, in some embodiments, also in the presence of exogenous cAMP-elevating agent. In some embodiments, the methods of the invention comprise the production of cultures of keratinocytes that comprise between about 90 and 99.99% keratinocytes, or between about 95 and 99.99% keratinocytes, or between about 98 and 99.99% keratinocytes, or between about 99 and 99.99% keratinocytes by culturing the keratinocytes in the presence of exogenous MMP-1 and, in some embodiments, also in the presence of exogenous cAMP-elevating agent.

Cells are passed when about 60-70% confluent. It is important for tissue culture not to let the cells grow to or near confluence, as cells at or near 100% confluence produce a fibrotic factor (TGF-beta) that encourages fibroblast growth. Thus, in some embodiments, cells are passaged when they are between about 40% to 90% confluent, or between about 50% to 80% confluent, or between about 50% to 70% confluent, or between about 60% to 70% confluent. In some embodiments, cells are passaged when they are about 50%, or 55%, or 60%, or 65%, or 70%, or 75%, or 80% confluent. In some cases, it may be wished to grow cells to greater degrees of confluence than 60-70% in order to obtain medium for certain therapeutic or other uses.

Cells can be passaged for at least two years. This is considerably different from normal keratinocyte culture, where, because of fibroblast proliferation, the cells must be used earlier. Some embodiments of the invention provide cultures that are at least about 90, 95, 98, 99, 99.5, 99.9, or 99.99% keratinocytes, where the culture persists for at least about 6 months, or about 9 months, or about 12 months, or about 15 months, or about 18 months, or about two years, or about two and one-half years, or about three years, or about four years, and where the cells are cultured for part or all of the culture period in medium that contains exogenous MMP-1 and, in some embodiments, exogenous cAMP-elevating agent.

V. Therapeutic and Cosmetic Compositions and Methods

A. Introduction

The present invention encompasses compositions and methods for enhancing wound healing by the use of a collagenase, e.g., in some embodiments MMP-1, and, optionally, a cAMP-elevating agent. The compositions and methods of the invention are also useful in enhancing the repair of other types of tissue damage, e.g., traumatic or congenital, wherein the repair and/or regeneration of tissue defects or damage is desired. The invention also provides cosmetic compositions and methods. In a further aspect, the invention provides compositions and methods for the treatment of cancer.

B. Wound Healing

In one aspect, the present invention relates to methods and compositions for treating a wound, and in particular, methods and compositions to promote and enhance wound healing. For example, in some embodiments, the present invention provides a method for treating a wound, comprising administering to a subject in need of wound treatment an effective amount of exogenous collagenase, e.g., MMP-1. In some embodiments an effective amount of an exogenous cAMP-elevating agent is also administered to the subject. The wound may be acute or chronic. In some embodiments, the administration is topical. In some embodiments, further compositions useful in wound healing are administered to the subject in combination with a collagenase, e.g., MMP-1 and, optionally, a cAMP-elevating agent.

Wound healing is a complex process that involves several stages and is capable of sealing breaches to the integument in a controlled manner to form functionally competent tissue. The process begins with hemostasis followed by an inflammatory phase involving neutrophils and macrophages. The process continues with the development of granulation tissue and re-epithelialization to close the wound. Subsequently, scar tissue forms and is remodeled over the succeeding months to an approximation of the original anatomical structure. Ideally, scar tissue is minimal so that healthy tissue, functionally competent tissue which histologically and physiologically resembles the original normal tissue, may form.

The primary goal in the treatment of wounds is to achieve wound closure, and one effect of the compositions and methods of the invention is to initiate, accelerate, complete, or otherwise optimize wound closure. Open cutaneous wounds represent one major category of wounds and include burn wounds, pressure sores, venous stasis ulcers, and diabetic ulcers. Numerous factors can affect wound healing, including malnutrition, infection, pharmacological agents (e.g., actinomycin and steroids), diabetes, and advanced age. Delayed wound healing causes substantial morbidity in patients with diabetes. Diabetes mellitus is a chronic disorder of glucose metabolism and homeostasis that damages many organs. It is a leading cause of death in the United States. In persons with diabetes, vascular disease, neuropathy, infections, and recurrent trauma predispose the extremities, especially the foot, to pathologic changes. These pathological changes can ultimately lead to chronic ulceration, which may necessitate amputation.

In addition, the compositions and methods of the invention can modulate the process of scar formation, including reducing or eliminating scarring, and normalizing scarring. It is well-known that skin from neonates heals from wounds with little scarring, and fetal skin heals with little or no scarring.

1. Definitions

A “wound,” as used herein, includes injuries to the skin or other epithelium and, in some cases, subcutaneous tissue, initiated in any way. Wounds are classified based on the depth of the wound; in the present invention, a “wound” refers to any breach of the epithelium, including partial or complete injury to the epidermis (Grade I); injury that extends to the dermis (Grade II); injury that extends to the subcutaneous tissue (Grade III); and full thickness wounds where bones are exposed (Grade IV). Non-exclusive examples of wounds include burn wounds, decubitus ulcers, venous-stasis ulcers, neuropathic ulcers, diabetic ulcers, poorly-healing wounds, normal surgical invasions, traumatic wounds, pyogenic granuloma, pyoderma gangrenosum, oral lesions, muscosal lesions, airway/lung lesions, gastric ulcerations, intestinal ulcerations, ulcerative colitis, Crohn's disease and opthalmic ulcerations. In addition, in some embodiments, wounds that may be treated by the present compositions and methods include gastric ulcers, pancreas, liver, kidney, spleen, blood vessel injuries and other internal wounds.

Wounds include, without limitation, wounds in which the skin is unbroken (contusions), wounds in which the skin is broken by a cutting instrument (incisions) and wounds in which the skin is broken by a dull or blunt instrument (lacerations). Wounds may be caused by accidents or by intentional acts such as surgical procedures. Wounds may also result from, or be related to a disease or disorder. For example, the wounds may be related to diabetes or cancer. The invention is particularly useful in the treatment of wounds related to diabetes, such as diabetic ulcers.

A “chronic wound,” as used herein, refers to a wound that has not healed within 30 days.

“Treatment” or “treating” as used herein includes achieving a therapeutic benefit and/or a prophylactic benefit. By therapeutic benefit is meant eradication or amelioration of the underlying disorder or condition being treated. For example, in an individual with a chronic wound, therapeutic benefit includes partial or complete closure of the wound, or any amelioration in the condition of the wound. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. A prophylactic benefit of treatment includes prevention of a condition, retarding the progress of a condition (e.g., slowing the progression of a chronic wound), or decreasing the likelihood of occurrence of a condition. As used herein, “treating” or “treatment” includes prophylaxis.

As used herein, the term “effective amount” can be an amount sufficient to effect beneficial or desired clinical results. An effective amount can be administered in one or more administrations. In terms of treatment, an “effective amount” of a composition of the invention is an amount that is sufficient to palliate, ameliorate, stabilize, reverse or slow the progression of a wound or other condition. An “effective amount” may be of a collagenase, e.g., MMP-1, -containing composition of the invention used alone or in conjunction with one or more agents used to treat a disease or disorder. An “effective amount” of a therapeutic agent within the meaning of the present invention will be determined by a patient's attending physician or veterinarian. Such amounts are readily ascertained by one of ordinary skill in the art and will enable enhanced wound healing when administered in accordance with the present invention. Factors which influence what a therapeutically effective amount will be include, the specific activity of the therapeutic agent being used, the wound type (mechanical or thermal, full or partial thickness, etc.), the size of the wound, the wound's depth (if full thickness), the absence or presence of infection, time elapsed since the injury's infliction, and the age, physical condition, existence of other disease states, and nutritional status of the patient. Additionally, other medication the patient may be receiving will affect the determination of the therapeutically effective amount of the therapeutic agent to administer.

A “subject” or an “individual,” as used herein, is an animal, for example, a mammal. In some embodiments a “subject” or an “individual” is a human. In some embodiments, the subject is a diabetic.

“Enhancing wound healing” or “promoting wound healing,” as used herein, refers to modulating wound healing in a desired manner. For example, enhancing or promoting wound healing includes initiating wound healing (e.g., in the case of non-healing chronic wounds), accelerating wound healing, providing more complete healing, allowing healing of a wound with less or no scarring, decreasing secondary wound characteristics such as pain, inflammation, danger of infection, bleeding, odor, and the like.

“Reduced scarring” refers to formation of less scar tissue during wound healing, or to the formation of more organized or less prominent or less visible scar tissue, than would likely occur without treatment. For example, reduced scarring can refer to the formation of a scar of smaller dimensions than would likely occur without treatment; reduced scarring can also refer to formation of a more organized or less prominent scar than would likely occur without treatment, e.g., to formation of a more nearly normal scar in an individual who is usually subject to keloid formation. “Scarless wound healing” or “healing without a scar” refers to healing of a wound substantially without scar that is visible to the naked eye, although microscopic healing or healing beneath the skin surface might still involve some degree of scarring.

The term “dressing” refers broadly to any material applied to a wound for protection, absorbance, drainage, to provide therapeutic substances to the wound, and the like. Numerous types of dressings are commercially available, including, but not limited to, films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer)

A “pharmaceutically acceptable carrier” herein refers to any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers and an inactive virus particle. Such carriers are well known to those of ordinary skill in the art. A thorough discussion of pharmaceutically acceptable excipients can be found in REMINGTON'S PHARMACEUTICALSCIENCES (Merck Pub. Co., N.J. 1991). Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like.

2. Compositions and Methods

The invention provides compositions and methods for treating wounds, in particular, for enhancing wound healing.

Compositions include exogenous collagenase, e.g., MMP-1, typically in combination with a pharmaceutically acceptable carrier. In some embodiments, compositions include exogenous collagenase, e.g., MMP-1 and a cAMP-elevating agent, typically in combination with a pharmaceutically acceptable carrier. In some embodiments, compositions include exogenous collagenase, e.g., MMP-1, a cAMP-elevating agent, and one or more other agents used in wound treatment, typically in combination with a pharmaceutically acceptable carrier. In some embodiments, collagenase, e.g., MMP-1 and a cAMP-elevating agent may be prepared in separate compositions, then used either separately or together, depending on the condition of the wound to be treated, such as the severity, depth, stage of healing, and the like. Such separate compositions allow one or the other of the compounds to be used, as well as differing ratios to be used, depending on the condition of the wound, as described. In addition, one or more other agents used in wound treatment may be similarly provided in a separate composition. The collagenase, e.g., MMP-1 and cAMP-elevating agent compositions, as well as compositions containing one or more other agents for wound treatment, if separately maintained, can be administered separately or contemporaneously.

The compositions of the invention also include one or more of the therapeutic agent(s) of the invention on a solid support. Therapeutic agents of the invention include exogenous collagenase, e.g., MMP-1, cAMP-elevating agents, and other agents for wound treatment. Solid supports include bandages, sutures, surgical staples, and the like. In another embodiment, active agents of the invention can be formulated into a skin covering or dressing containing a therapeutically effective amount of one or more active agents impregnated into, covalently attached or otherwise associated with a covering or dressing material. In one embodiment, the skin covering or dressing permits release of the active agents.

Release of the active agents can be in an uncontrolled or a controlled manner.

In another embodiment, the invention is directed to a method of preparing a therapeutic pharmaceutical composition for treatment of wounds, which comprises admixing a therapeutically effective amount of a therapeutic wound healing composition containing exogenous collagenase, e.g., MMP-1 with a pharmaceutically acceptable carrier to form a therapeutic pharmaceutical composition. In some embodiments the method comprises admixing a therapeutically effective amount of a therapeutic wound healing composition containing exogenous collagenase, e.g., MMP-1 and a cAMP-elevating agent with a pharmaceutically acceptable carrier to form a therapeutic pharmaceutical composition. In some embodiments the method comprises admixing a therapeutically effective amount of a therapeutic wound healing composition containing exogenous collagenase, e.g., MMP-1, a cAMP-elevating agent, and one or more other agents for wound treatment with a pharmaceutically acceptable carrier to form a therapeutic pharmaceutical composition.

Direct delivery of the compositions will generally be accomplished by oral and pulmonary administration, suppositories, and transdermal applications. The wound to which the therapeutic combinations are applied can be internal or external, and may be directed towards any tissue exhibiting a wound, for example epithelial tissue. Other modes of administration include injection, either subcutaneously, intradermally, intraperitoneally, intraluminally, intragastrically, intraintestinally, intravenously or intramuscularly. Dosage treatment may be a single dose schedule or a multiple dose schedule. Administration of the therapeutic combinations of the invention can be accomplished by, for example, topical cream, foam, injection, aerosol spray, in a gel matrix, a sponge, drops, and a wash. Administration can be by, for example, local, oral, intradermal, subcutaneous, intraluminal, intragastric, and intraperitoneal administration with an appropriate formulation of the selected composition made up of a combination of the therapeutics appropriate for a particular treatment.

Methods of the invention include the administration to a subject with a wound of an effective amount of an exogenous collagenase, e.g., MMP-1, optionally in combination with a cAMP-elevating agent. Thus, in some embodiments of the invention the subject is administered an effective amount of exogenous collagenase, MMP-1. In some embodiments of the invention the subject is administered an effective amount of collagenase, e.g., MMP-1 and a cAMP-elevating agent. In some embodiments of the invention the subject is administered an effective amount of exogenous collagenase, e.g., MMP-1 in combination with one or more other agents used in wound treatment. In some embodiments of the invention the subject is administered an effective amount of MMP-1 and a cAMP-elevating agent in combination with one or more other agents used in wound healing. In some embodiments, the administration is topical administration. In some embodiments, the administration is via injection, e.g., subcutaneous, dermal, intramuscular, or intraperitoneal injection. In some embodiments, the administration is to the oral mucosa. In some embodiments, the administration is via suppository.

Compositions

“Therapeutic agent,” as used herein, refers to collagenase, e.g., exogenous MMP-1, a cAMP-elevating agent, and/or other agents used in wound treatment. Sources and types of collagenase, e.g., MMP-1, and cAMP-elevating agents are as described above. Other agents used in wound treatment include those described herein. Amounts and concentrations of each agent are known in the art and the appropriate dosage may be found by no more than routine experimentation.

While it is possible to administer the therapeutic agent as a pure or substantially pure compound, i.e. not incorporated into any pharmaceutical composition, it is preferable instead to present the therapeutic agent in a pharmaceutical formulation or composition. Such compositions comprise a therapeutically effective amount of the therapeutic agent with one or more pharmaceutically acceptable carriers.

Thus, in some embodiments, the invention provides a composition for treatment of wounds that comprises:

A) a therapeutically effective amount of exogenous collagenase, and, optionally

B) a pharmaceutically acceptable carrier.

In some embodiments, the invention provides a composition for treatment of wounds that comprises:

A) a first amount of exogenous collagenase;

B) a second amount of a cAMP-elevating agent which, in combination with the first amount of collagenase provided, provides a therapeutic effect; and

C) a pharmaceutically acceptable carrier.

In some of these embodiments, the collagenase is MMP-1; in some embodiments the MMP-1 is human MMP-1; and in some embodiments, the human MMP-1 is recombinant human MMP-1. In some embodiments, the cAMP-elevating agent is selected from the group consisting of forskolin, dibutyryl cAMP, Isobutylmethylxanthine (IBMX), Theophylline, isoproterenol, and PGE2.

In some embodiments, the invention provides a composition for treatment of wounds that comprises:

A) a first amount of exogenous collagenase;

B) a second amount of cAMP-elevating agent which, in combination with the first amount of collagenase provided, provides a therapeutic effect;

Optionally, C) one or more additional agents used for wound treatment and

D) a pharmaceutically acceptable carrier.

In some of these embodiments, the collagenase is MMP-1; in some embodiments the MMP-1 is human MMP-1; and in some embodiments, the human MMP-1 is recombinant human MMP-1. In some embodiments, the collagenase is bacterial collagenase, e.g., highly purified low-endotoxin bacterial collagenase, or essentially endotoxin-free bacterial collagenase. Bacterial collagenases are described in reference to cell culture and media. In some embodiments, the cAMP-elevating agent is selected from the group consisting of forskolin, dibutyryl cAMP, Isobutylmethylxanthine (IBMX), Theophylline, isoproterenol, and PGE2.

Pharmaceutical compositions of the invention include compositions suitable for non-oral topical use, and/or systemic use, e.g., oral or parenteral use.

The invention also provides methods for making a composition for the treatment of wounds. All composition methods include the step of bringing the therapeutic agent(s) into association with the carrier(s).

Nonexclusive examples of pharmaceutically compositions include pharmaceutical appliances, non-oral topical compositions, oral topical compositions, compositions for ingestion, and injectable compositions. Examples of pharmaceutical appliances are sutures, staples, gauze, bandages, burn dressings, artificial skins, liposome or micelle formulations, microcapsules, aqueous vehicles for soaking gauze dressings, and the like, and mixtures thereof. Non-oral topical compositions employ non-oral topical vehicles, such as creams, gels, formulations, foams, ointments and sprays, salves, and films, which are intended to be applied to the skin or body cavity and are not intended to be taken by mouth. Oral topical compositions employ oral vehicles, such as mouthwashes, rinses, oral sprays, suspensions, and dental gels, which are intended to be taken by mouth but are not intended to be ingested. Ingestible compositions employ ingestible or partly ingestible vehicles. Injectable compositions employ liquid vehicles, usually an aqueous vehicle.

In some embodiments, the invention provides one or more therapeutic agents of the invention incorporated into a non-oral topical vehicle which may be in the form of a powder, lotion, cream, gel, foam, ointment, spray, salve, tincture, jelly, paste, suppository or solution. For salves and creams, traditional binders, carriers and excipients may include, for example, polyalkylene glycols or triglycerides.

Topical formulations include, but are not limited to ointments, creams, gels, and biodegradable polymers. Ointments generally are prepared using either (1) an oleaginous base, i.e., consisting of fixed oils or hydrocarbons, such as white petrolatum or mineral oil, or (2) an absorbent base, i.e., consisting of an anhydrous substance or substances that can absorb water, for example anhydrous lanolin. Customarily, following formation of the base, whether oleaginous or absorbent, the active ingredient (compound) is added to an amount affording the desired concentration. In some embodiments, the ointment is petrolatum, e.g., white petrolatum, and the components are admixed with the petrolatum.

Creams are oil/water emulsions. They consist of an oil phase (internal phase), typically comprising fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate; hydrophilic colloids, such as acacia colloidal clays, veegum, and the like. Upon formation of the emulsion, the active ingredient (compound) customarily is added in an amount to achieve the desired concentration.

Gels comprise a water-insoluble material, where he water-insoluble material forms a gel with the water of the formulation. The material is therefore hydrophilic but does not dissolve in water to any great extent. The material can be a polymeric material, for example, a water-absorbing non water-soluble polymer.

Gels typically comprise a base selected from an oleaginous base, water, or an emulsion-suspension base. To the base is added a gelling agent which forms a matrix in the base, increasing its viscosity. Examples of gelling agents are polymers such as hydroxypropyl cellulose, acrylic acid polymers, and the like. However, non-polymeric materials that form gels with water can also be used, e.g. clays such as kaolin or bentonite. Customarily, the active ingredient (compounds) is added to the formulation at the desired concentration at a point preceding addition of the gelling agent. The amount of compound incorporated into a topical formulation is not critical; the concentration should be within a range sufficient to permit ready application of the formulation to the affected tissue area in an amount that will deliver the desired amount of compound to the desired treatment site. The customary amount of a topical formulation to be applied to an affected tissue will depend upon an affected tissue size and concentration of compound in the formulation.

In some embodiments, one or more other agents for wound treatment may also be encapsulated in a biodegradable polymer. Biodegradable polymers are usually based on functional groups such as esters, anhydrides, orthoesters, and amides. Rapidly biodegradable polymers include poly[lactide-co-glycolide], polyanhydrides, and polyorthoesters. Preferred bioerodible polymers include polylactides, polyglycolides, and copolymers thereof, poly(ethylene terephthalate), poly(butyric acid), poly(valeric acid), poly(lactide-co-caprolactone), poly(lactide-co-glycolide), polyanhydrides, polyphosphazenes, poly(ε-caprolactone), poly(dioxanone), poly(hydroxybutyrate), poly(hydroxyvalerate), polyorthoesters, blends, and copolymers thereof. Examples of biodegradable and biocompatible polymers of acrylic and methacrylic acids or esters include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), etc. Other polymers which can be used in the present invention include polyalkylenes such as polyethylene and polypropylene; polyarylalkylenes such as polystyrene; poly(alkylene glycols) such as poly(ethylene glycol); poly(alkylene oxides) such as poly(ethylene oxide); and poly(alkylene terephthalates) such as poly(ethylene terephthalate). Additionally, polyvinyl polymers can be used which include polyvinyl alcohols, polyvinyl ethers, polyvinyl esters, and polyvinyl halides. Exemplary polyvinyl polymers include poly(vinyl acetate), polyvinyl phenol, and polyvinylpyrrolidone. Mixtures of two or more of the above polymers could also be used in the present invention

Some polymeric materials are known to release entrapped compounds upon exposure to a stimulus such as a change in pH or temperature. An examples of microparticles that release as a function of a change in pH include the diketopiperazine particles describes in U.S. Pat. No. 5,352,461 issued on Oct. 4, 1994, to Steiner et al., and the proteinoid formulations described in U.S. Pat. Reissue No. 35,862, issued on Jul. 28, 1998.

The non-oral topical compositions may also contain conventional additives employed in those products. Conventional additives include humectants, emollients, lubricants, stabilizers, dyes and other coloring agents, and perfumes, providing the additives do not interfere with the therapeutic properties of the composition.

In accordance with this invention, therapeutically effective amounts of the compositions of the present invention may be admixed with a non-oral topical vehicle to form a topical composition. These amounts are readily determined by those skilled in the art without the need for undue experimentation.

In some embodiments, the non-oral topical compositions comprise exogenous collagenase, e.g., MMP-1 such as human MMP-1, in an amount from about 0.01% to about 10%. In some embodiments, the non-oral topical compositions comprise exogenous collagenase, e.g., human MMP-1, in an amount from about 0.01% to about 5%. In some embodiments, the non-oral topical compositions comprise exogenous collagenase, e.g., human MMP-1, in an amount from about 0.01% to about 2%. In some embodiments, the non-oral topical compositions comprise exogenous collagenase, e.g., human MMP-1, in an amount from about 0.01% to about 1%. In some embodiments, the non-oral topical compositions comprise exogenous collagenase, e.g., human MMP-1, in an amount from about 0.1% to about 10%. In some embodiments, the non-oral topical compositions comprise exogenous collagenase, e.g., human MMP-1, in an amount from about 0.1% to about 5%. In some embodiments, the non-oral topical compositions comprise exogenous collagenase, e.g., human MMP-1, in an amount from about 0.1% to about 2%. In some embodiments, the non-oral topical compositions comprise exogenous collagenase, e.g., human MMP-1, in an amount from about 0.1% to about 1%. In some embodiments, the collagenase present as MMP-1 is present in an amount of about 0.1-100, 1-100 k 5-50, or 10-40 mg/gm. In some embodiments, collagenase activity is measured in U/gm of composition. In these embodiments, collagenase may be present at a concentration of about 1-10,000, 10-1000, 20-500, or 50-200 U/gm.

In some embodiments, in addition to exogenous collagenase at the concentration described, the non-oral topical composition comprises a cAMP-elevating agent in an amount from about 0.01% to about 10%. In some embodiments, the non-oral topical compositions comprise a cAMP-elevating agent in an amount from about 0.01% to about 5%. In some embodiments, the non-oral topical compositions comprise a cAMP-elevating agent in an amount from about 0.01% to about 2%. In some embodiments, the non-oral topical compositions comprise a cAMP-elevating agent in an amount from about 0.01% to about 1%. In some embodiments, the non-oral topical compositions comprise a cAMP-elevating agent in an amount from about 0.1% to about 10%. In some embodiments, the non-oral topical compositions comprise a cAMP-elevating agent in an amount from about 0.1% to about 5%. In some embodiments, the non-oral topical compositions comprise a cAMP-elevating agent in an amount from about 0.1% to about 2%. In some embodiments, the non-oral topical compositions comprise a cAMP-elevating agent in an amount from about 0.1% to about 1%.

In some instances, a ratio of collagenase, e.g., MMP-1 and cAMP elevating agent is used, such as, e.g., a molar ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 mole:mole. In some embodiments, a weight ratio is used, such as, e.g., a weight ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 wt:wt. In some embodiments the ratio is of U/gm collagenase activity to ug/gm cAMP-elevating agent. In these embodiments, the ratio may be, e.g., about 1 U/gm collagenase activity to about 10 mg/gm cAMP-elevating agent, or about 1 U/gm collagenase activity to about 1 mg/gm cAMP-elevating agent, or about 1 U/gm collagenase activity to about 0.1 mg/gm cAMP-elevating agent, or about 1 U/gm collagenase activity to about 100 mg/gm cAMP-elevating agent, or about 0.1 U/gm collagenase activity to about 100 mg/gm cAMP-elevating agent. In some embodiments the ratio is about 10 units/gm to about 1 mg/gm.

In some embodiments the invention provides a composition to enhance wound healing comprising highly purified low-endotoxin collagenase at a concentration of about 120 U/gm and forskolin at about 12.5 mg/gm, in an ointment base. In some embodiments, the ointment base is white petrolatum.

In some embodiments, in addition to exogenous collagenase at the concentration described, and, optionally, a cAMP-elevating agent at the concentration described, the non-oral topical composition comprises one or more additional agents used for wound treatment in an amount from about 0.01% to about 10%. In some embodiments, the non-oral topical compositions comprise one or more additional agents used for wound treatment in an amount from about 0.01% to about 5%. In some embodiments, the non-oral topical compositions comprise one or more additional agents used for wound treatment in an amount from about 0.01% to about 2%. In some embodiments, the non-oral topical compositions comprise one or more additional agents used for wound treatment in an amount from about 0.01% to about 1%. In some embodiments, the non-oral topical compositions comprise one or more additional agents used for wound treatment in an amount from about 0.1% to about 10%. In some embodiments, the non-oral topical compositions comprise one or more additional agents used for wound treatment in an amount from about 0.1% to about 5%. In some embodiments, the non-oral topical compositions comprise one or more additional agents used for wound treatment in an amount from about 0.1% to about 2%. In some embodiments, the non-oral topical compositions comprise one or more additional agents used for wound treatment in an amount from about 0.1% to about 1%.

The present invention extends to methods for preparing the non-oral topical compositions. In such a method, the non-oral topical composition is prepared by admixing a therapeutically effective amount of exogenous MMP-1, optionally with a cAMP-elevating agent and/or another agent for wound treatment a non-oral topical vehicle. The final compositions are readily prepared using standard methods and apparatus generally known by those skilled in the pharmaceutical arts.

In another form of the invention, the therapeutic wound healing composition is incorporated into an oral topical vehicle which may be in the form of a mouthwash, rinse, oral spray, suspension, dental gel, and the like. Typical nontoxic oral vehicles known in the pharmaceutical arts may be used in the present invention. The preferred oral vehicles are water, ethanol, and water-ethanol mixtures. The water-ethanol mixtures are generally employed in a weight ratio from about 1:1 to about 20:1, preferably from about 3:1 to about 20:1, and most preferably from about 3:1 to about 10:1, respectively. The pH value of the oral vehicle is generality from about 4 to about 7, and preferably from about 5 to about 6.5. An oral topical vehicle having a pH value below about 4 is generally irritating to the oral cavity and an oral vehicle having a pH value greater than about 7 generally results in an unpleasant mouth feel.

The oral topical therapeutic wound healing compositions may also contain conventional additives normally employed in those products. Conventional additives include fluorine providing compound, a sweetening agent, a flavoring agent, a coloring agent, a humectant, a buffer, and an emulsifier, providing the additives do not interfere with the therapeutic properties of the therapeutic wound healing composition.

Suitable buffer solutions useful in the oral topical therapeutic wound healing compositions include citric acid-sodium citrate solution, phosphoric acid-sodium acetate solution in amounts up to about 1%, and preferably from about 0.05% to about 0.5% by weight of the oral topical therapeutic wound healing composition.

In some embodiments, the oral topical compositions comprise exogenous MMP-1 in an amount from about 0.01% to about 10%. In some embodiments, the oral topical compositions comprise exogenous MMP-1 in an amount from about 0.01% to about 5%. In some embodiments, the oral topical compositions comprise exogenous MMP-1 in an amount from about 0.01% to about 2%. In some embodiments, the oral topical compositions comprise exogenous MMP-1 in an amount from about 0.01% to about 1%. In some embodiments, the oral topical compositions comprise exogenous MMP-1 in an amount from about 0.1% to about 10%. In some embodiments, the oral topical compositions comprise exogenous MMP-1 in an amount from about 0.1% to about 5%. In some embodiments, the oral topical compositions comprise exogenous MMP-1 in an amount from about 0.1% to about 2%. In some embodiments, the oral topical compositions comprise exogenous MMP-1 in an amount from about 0.1% to about 1%. In some embodiments, the collagenase present as MMP-1 is present in an amount of about 0.1-100, 1-100 k 5-50, or 10-40 mg/gm. In some embodiments, collagenase activity is measured in U/gm of composition. In these embodiments, collagenase may be present at a concentration of about 1-10,000, 10-1000, 20-500, or 50-200 U/gm.

In some embodiments, in addition to collagenase, e.g., MMP-1 at the concentration described, the oral topical composition comprises a cAMP-elevating agent in an amount from about 0.01% to about 10%. In some embodiments, the oral topical compositions comprise a cAMP-elevating agent in an amount from about 0.01% to about 5%. In some embodiments, the oral topical compositions comprise a cAMP-elevating agent in an amount from about 0.01% to about 2%. In some embodiments, the oral topical compositions comprise a cAMP-elevating agent in an amount from about 0.01% to about 1%. In some embodiments, the oral topical compositions comprise a cAMP-elevating agent in an amount from about 0.1% to about 10%. In some embodiments, the oral topical compositions comprise a cAMP-elevating agent in an amount from about 0.1% to about 5%. In some embodiments, the oral topical compositions comprise a cAMP-elevating agent in an amount from about 0.1% to about 2%. In some embodiments, the oral topical compositions comprise a cAMP-elevating agent in an amount from about 0.1% to about 1%.

In some instances, a ratio of collagenase, e.g., MMP-1 and cAMP elevating agent is used, such as, e.g., a molar ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 mole:mole. In some embodiments, a weight ratio is used, such as, e.g., a weight ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 wt:wt.

In some embodiments, in addition to collagenase, e.g., MMP-1 at the concentration described, and, optionally, a cAMP-elevating agent at the concentration described, the oral topical composition comprises one or more additional agents used for wound treatment in an amount from about 0.01% to about 10%. In some embodiments, the oral topical compositions comprise one or more additional agents used for wound treatment in an amount from about 0.01% to about 5%. In some embodiments, the oral topical compositions comprise one or more additional agents used for wound treatment in an amount from about 0.01% to about 2%. In some embodiments, the oral topical compositions comprise one or more additional agents used for wound treatment in an amount from about 0.01% to about 1%. In some embodiments, the oral topical compositions comprise one or more additional agents used for wound treatment in an amount from about 0.1% to about 10%. In some embodiments, the oral topical compositions comprise one or more additional agents used for wound treatment in an amount from about 0.1% to about 5%. In some embodiments, the oral topical compositions comprise one or more additional agents used for wound treatment in an amount from about 0.1% to about 2%. In some embodiments, the oral topical compositions comprise one or more additional agents used for wound treatment in an amount from about 0.1% to about 1%.

The present invention extends to methods for preparing the oral topical compositions. In such a method, the oral topical therapeutic wound healing composition is prepared by admixing a therapeutically effective amount of exogenous MMP-1, optionally with a cAMP-elevating agent and/or another agent for wound treatment and an oral topical vehicle. The final compositions are readily prepared using standard methods and apparatus generally known by those skilled in the pharmaceutical arts.

In some embodiments, the invention provides one or more therapeutic agents of the invention incorporated into an ingestible vehicle. The vehicle may be a solid, semisolid or liquid material that acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, or suppositories, described in more detail below. Any suitable vehicle known in the art may be used in the pharmaceutical compositions of the invention. See, e.g., See, e.g. Remington's Pharmaceutical Sciences, Gennaro, A R, ed., 20^{th} edition, 2000: Williams and Wilkins PA, USA. A pharmaceutically-acceptable vehicle within the scope of the present invention meets industry standards for sterility, isotonicity, stability, and non-pyrogenicity. (Remington's Pharmaceutical Sciences, supra), in the form of lyophilized cake or aqueous solutions. Acceptable vehicles, excipients, and stabilizers are nontoxic to the cell or mammal being exposed at the dosages and concentrations employed. Examples include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The present invention extends to methods of making the ingestible therapeutic wound healing compositions. In such methods, an ingestible therapeutic wound healing composition is prepared by admixing a therapeutically effective amount of the therapeutic wound healing composition with a pharmaceutically-acceptable carrier. The apparatus useful in accordance with the present invention comprises mixing and heating apparatus well known in the confectionery arts, and therefore the selection of the specific apparatus will be apparent to the artisan. The final ingestible therapeutic wound healing compositions are readily prepared using methods generally known in the confectionery arts.

In one form of the invention, the therapeutic wound healing composition is incorporated into a pharmaceutical appliance which may be in the form of sutures, staples, gauze, bandages, burn dressings, artificial skins, liposome or micelle formulations, microcapsules, aqueous vehicles for soaking gauze dressings, and the like, and mixtures thereof. A variety of traditional ingredients may optionally be included in the pharmaceutical composition in effective amounts such as buffers, preservatives, toxicity adjusting agents, antioxidants, polymers for adjusting viscosity or for use as extenders, and excipients, and the like. Specific illustrative examples of such traditional ingredients include acetate and borate buffers, thimerosal, sorbic acid, methyl and propyl paraben and colorobutanol preservatives; sodium chloride and sugars to adjust the toxicity; and excipients such as mannitol, lactose and sucrose. Other conventional pharmaceutical additives known to those having ordinary skill in the pharmaceutical arts may also be used in the pharmaceutical composition.

In some embodiments the invention provides one or more therapeutic agents associated with a solid support. In some embodiments, the solid support is a skin covering or wound dressing. In some embodiments, the solid support is an absorbent material, e.g., attached to an adhesive strip. Thus, in some embodiments, Hence, the skin coverings or wound dressings of the invention can provide slow or timed release of the active agents, i.e., MMP-1, optionally with a cAMP-elevating agent, and, in some embodiments, further including additional therapeutic agents, into a wound. Skin coverings and dressing materials can be any material used in the art including bandage, gauze, sterile wrapping, hydrogel, hydrocolloid and similar materials.

In accordance with this invention, therapeutically effective amounts of the therapeutic wound healing compositions of the present invention may be employed in the pharmaceutical appliance. These amounts are readily determined by those skilled in the art without the need for undue experimentation. The exact amount of therapeutic wound healing composition employed is subject to such factors as the type and concentration of the therapeutic wound healing composition and the type of pharmaceutical appliance employed. Thus, the amount of therapeutic wound healing composition will be varied in order to obtain the result desired in the final product and such variations are within the capabilities of those skilled in the art without need for undue experimentation. In a preferred embodiment, the pharmaceutical composition will comprise the therapeutic wound healing composition in an amount from about 0.01% to about 100%, by weight of the pharmaceutical composition. In a more preferred embodiment, the pharmaceutical composition will comprise the therapeutic wound healing composition in an amount of about 0.1% to about 25%, by weight of the pharmaceutical composition. In a most preferred embodiment, the pharmaceutical composition will comprise the therapeutic wound healing composition in an amount of about 0.1% to about 15%, by weight of the pharmaceutical composition.

The present invention extends to methods for making the pharmaceutical compositions. In general, a pharmaceutical composition is made by contacting a therapeutically effective amount of a therapeutic wound healing composition with the pharmaceutical appliance and the other ingredients of the final desired pharmaceutical composition. The therapeutic wound healing composition may be absorbed onto a pharmaceutical appliance.

Other ingredients will usually be incorporated into the composition as dictated by the nature of the desired composition as well known by those having ordinary skill in the art. The ultimate pharmaceutical compositions are readily prepared using methods generally known in the pharmaceutical arts. ions may contain adjunct materials employed in formulating the suspensions of the art.

The compositions are also prepared as injectables, either as liquid solutions or suspensions; solid. Thus, the therapeutic agent(s) may be systemically administered, for example, intravenously or intraperitoneally by infusion or injection. Solutions of the therapeutic agent(s) can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The pharmaceutical dosage forms suitable for injection or infusion or topical application can include sterile aqueous solutions or dispersions or sterile powders comprising the active ingredient that are adapted for the extemporaneous preparation of sterile injectable or infusible solutions or dispersions, optionally encapsulated in liposomes.

In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like.

In some cases, one of skill in the art may choose to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the therapeutic agent(s) in the required amount in the appropriate solvent with various of the other ingredients enumerated above, as required, followed by filter sterilization. In the case of sterile powders for the preparation of sterile injectable solutions, methods of preparation include vacuum drying and the freeze-drying techniques, which yield a powder of the active ingredient plus any additional desired ingredient present in the previously sterile-filtered solutions.

The formulation may be packaged into tubes, tubs or other suitable forms of containers for storage or it may be spread onto a substrate and then subsequently packaged. Suitable substrates include dressings, including film dressings, and bandages.

Therapeutic agents of the invention to be used for in vivo administration are preferably sterile. This is readily accomplished by any method known in the art, such as filtration through sterile filtration membranes, prior to or following lyophilization and reconstitution.

The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

Additional therapeutic agents. Additional ingredients useful in compositions for enhanced wound healing include initiators and enhancers of wound healing, antiinflammatory agents, antiviral agents, antimicrobial agents, anesthetics and analgesics, antipruritics, and vitamins and antioxidants.

Initiators and Enhancers of Wound Healing MMP-1 and/or cAMP may be combined with an agent to enhance wound healing. For example, initiators of wound healing include, but are not limited to keratinocyte growth factor (KGF), platelet derived growth factor (PDGF); basic fibroblast growth factor (bFGF), acidic fibroblast growth factor (aFGF), epidermal growth factor (EGF), transforming growth factor-alpha (TGF-α), transforming growth factor-beta (TGF-β), neu differentiation factor (rNDF), insulin-like growth factor I (IGF-1), heparin-binding epidermal growth factor (HB-EGF) and insulin-like growth factor II (IGF-II). See, e.g., U.S. Pat. No. 4,861,757

Additionally, other growth factors, e.g., recombinant growth factors, include insulin, Interferons (IFNs), Interleukins (ILs), Macrophage Colony Stimulating Factor (M-CSF), Platelet-Derived Endothelial Cell Growth Factor (PD-ECGF), and Stem Cell Factor (SCF), any or all of which may promote the activation, proliferation, and/or stimulation of cell types involved in the wound healing process.

Additional growth factors and other factors that enhance wound healing, and which may be of use in the invention, include those described in, e.g., U.S. Pat. Nos. 6,903,078; 6,841,355; 6,838,430; 6,808,707; 6,767,891; 6,689,351; 6,670,337; 6,660,306; 6,638,909; all of which are incorporated herein by reference in their entirety.

Antiinflammatory agents Anti-inflammatory agents of use in the invention include steroidal, non-steroidal, and other compounds.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Nonsteroidal anti-inflammatory agents are also suitable for use herein as skin agents in the compositions of the invention. Non-limiting examples of non-steroidal anti-inflammatory agents suitable for use herein include oxicams (e.g., piroxicam, isoxicam, tenoxicam, sudoxicam, CP-14,304); salicylates (e.g., aspirin, disalcid, benorylate, trilisate, safapryn, solprin, diflunisal, fendosal); acetic acid derivatives (e.g., diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac); fenamates (e.g., mefenamic, meclofenamic, flufenamic, niflumic, tolfenamic acids); propionic acid derivatives (e.g., ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic); pyrazoles (e.g., phenylbutazone, oxyphenbutazone, feprazone, azapropazone, trimethazone); and combinations thereof as well as any dermatologically acceptable salts or esters of thereof. COX-2 inhibitors are also suitable for use herein, and include, but are not limited to, AZD 3582 (ASTRAZENECA and NicOx), Celecoxib (PHARMACIA Corp.) (4-[5-(4-methylphenyl)-3-(trifluoromethyl)-1H-pyrazol-1-yl]benzenesulfonamide), Meloxicam (BOEHRINGER INGELHEIM Pharmaceuticals) (4-hydroxy-2-methyl-N-(5-methyl-2-thiazolyl)-2H-1,2GW-406381 (GLAXOSMITHKLINE), Etoricoxib (MERCK & Co.), Rofecoxib (MERCK & Co.) (4-[4-(methylsulfonyl)phenyl]-3-phenyl-2(5H)-furanone), Lumiracoxib (NOVARTIS Pharma AG), Valdecoxib (PHARMACIA Corp.) (4-(5-methyl-3-phenyl-4-isox-azolyl)benzenesulfonamide), and Etodolac (WYETH Ayerst Laboratories) ((.+−.) 1,8-diethyl-1,3,4,9-tetrahydropyrano-[3,4-b]acid).

Other non-limiting examples of suitable anti-inflammatory or similar other agents include candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, sea whip extract, anise oil, garlic oil, ginger extract, vasoconstrictors such as phenylephrine hydrochloride, and combinations thereof.

Further non-limiting examples of suitable anti-inflammatory or similar agents include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific non-limiting examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, and combinations thereof.

Antiviral agents Compositions and methods of the invention may include antiviral agents. Suitable anti-viral agents include, but are not limited to, metal salts (e.g., silver nitrate, copper sulfate, iron chloride, etc.) and organic acids (e.g., malic acid, salicylic acid, succinic acid, benzoic acid, etc.).

Antimicrobials Anti-microbial agents useful in compositions and methods of the invention include antifungal, antibacterial, and antiseptic compounds.

Antifungal compounds include, but are not limited to, compounds such as imidazole antifungals. Specific antifungals include butocouazole nitrate, miconazole, econazole, ketoconazole, oxiconizole, haloprogin, clotrimazole, and butenafine HCl, naftifine, terbinafine, ciclopirox, and tolnaftate.

Antibacterial and antiseptic compounds include phenol-TEA complex, mupirocin, triclosan, chlorocresol, chlorbutol, iodine, clindamycin, CAE (Anjinomoto Co., Inc., containing DL-pyrrolidone Carboxylic acid salt of L-Cocoyl Arginine Ethyl Ester), povidone-iodine, polymyxin b sulfate-bacitracin, zinc-neomycin sulfate-hydrocortisone, chloramphenicol, methylbenzethonium chloride, and erythromycin and antiseptics (e.g., benzalkonium chloride, benzethonium chloride, chlorhexidine gluconate, mafenide acetate, nitrofurazone, nitromersol and the like may be included in compositions of the invention. Antiparasitics, such as lindane may also be included.

Further examples of antimicrobial and antifungal agents useful in the present invention include, but are not limited to, β-lactam drugs, quinolone drugs, ciprofloxacin, norfloxacin, tetracycline, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole.

In particular, topical antibiotics suitable for use in the invention chloramphenicol, chlortetracycline, clyndamycin, clioquinol, erythromycin, framycetin, gramicidin, fusidic acid, gentamicin, mafenide, mupiroicin, neomycin, polymyxin B, bacitracin, silver sulfadiazine, tetracycline and chlortetracycline.

In addition, in some embodiments it may be desirable to administer systemic antibiotics in combination with administration of a composition of the invention. Any suitable systemic antibiotic known in the art and suitable for use with a particular individual being treated may be used in combination with the compositions of the invention.

Anesthetics and Analgesics Anesthetic substances of use in the invention include butamben picrate, lidocaine, xylocaine, benzocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Analgesic agents include dyclonine hydrochloride, aloe vera, fentanyl, capsaicin, and the like.

Antipruritics Anti-pruritic agents include alclometasone dipropionate, betamethasone valerate, and isopropyl myristate MSD.

Vitamins and antioxidants Vitamins and antioxidants may also be used in combination with the compositions and methods of the invention. These include vitamins K, E, and C. Addition of vitamin K may promote wound healing, and the antioxidant vitamins C and E also have beneficial effects on wound healing and may reduce scar formation. See, e.g., U.S. Pat. No. 6,187,743.

The compositions and methods of the present invention may utilize a wide range of additional components. The CTFA Cosmetic Ingredient Handbook, Seventh Edition, 1997 and the Eighth Edition, 2000, which are incorporated by reference herein in its entirety, describes a wide variety of ingredients commonly used in skin care compositions and methods, which are suitable for use in the compositions of the present invention. Other topically-useful compounds are listed in Remington's Pharmaceutical Sciences, 20th Ed., Lippincott Williams & Witkins, Baltimore, Md. (2000) (hereinafter Remington's), U.S. Pharmacopeia and National Formulary, The United States Pharmacopeial Convention, Inc., Rockville, Md. and Physician's Desk Reference, Medical Economics Co., Inc., Oradell, N.J. incorporated herein by reference. The concentration of the other ingredient in formulations provided by the invention is that which provides an effective amount of the other ingredient; these concentrations are well-known in the art. See, e.g., the above references, as well as Textbook of Dermatology, Champion, Burton, Burns, and Bretnach, eds., Blackwell Publishing, 1998.

Methods

The compositions according to the invention can be administered in any circumstance in which wound healing is desired.

These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts. In the methods described herein, a subject to be treated can be any animal, e.g., a mammal, so long as the animal, e.g., mammal has a wound that is in need of healing. In a preferred embodiment the subjects are human subjects. However, the present methods may also find particular use in the treatment of wounds in domestic animals.

In some embodiments, exogenous collagenase, e.g., MMP-1 is administered to a mammal in an amount effective to enhance the healing of a wound. In some embodiments, the collagenase, e.g. human MMP-1, is recombinant. In some embodiments, the mammal is a human. In some embodiments exogenous collagenase, e.g., human MMP-1 is administered as a lyophilized powder. More typically, the collagenase, e.g., exogenous human MMP-1 is administered as a pharmaceutical composition as described herein. The collagenase, e.g., exogenous human MMP-1 may be administered systemically. In some embodiments, the collagenase, e.g., exogenous human MMP-1 is administered directly to the site of the wound, such as by topical administration.

In some embodiments, exogenous collagenase in combination with a cAMP-elevating agent is administered to a mammal in an amount effective to enhance the healing of a wound. In some embodiments, the mammal is a human. In some embodiments, the exogenous collagenase is MMP-1, e.g., human MMP-1 such as recombinant human MMP-1. In some embodiments, the cAMP-elevating agent is forskolin. In one embodiment either or both of the exogenous collagenase and the cAMP-elevating agent is administered as a lyophilized powder. More typically, both the exogenous collagenase and the cAMP-elevating agent are administered as one or more pharmaceutical compositions as described herein. Either or both of the exogenous collagenase and the cAMP-elevating agent may be administered systemically. More typically, both are administered directly to the site of the wound, such as by topical administration.

In some embodiments, one or more additional agents for wound treatment are administered in addition to collagenase and a cAMP-elevating agent. Depending on the nature of the additional agent or agent, administration may be systemic, directly to the wound site, or a combination. For example, antimicrobials (antibiotics, antifungals, and the like) may be administered both systemically and to the wound site. Additional agents are described above.

The therapeutics of the invention can be administered in a therapeutically effective dosage and amount, in the process of a therapeutically effective protocol for treatment of the patient. The initial and any subsequent dosages administered will depend upon the patient's age, weight, condition, and the disease, wound, disorder or biological condition being treated. Depending on the therapeutic, the dosage and protocol for administration will vary, and the dosage will also depend on the method of administration selected, for example, local or systemic administration. The doses for a particular wound will be determined on a patient by patient basis, and depend on the size of the wound, the type of injury, and the composition that is applied. Doses for the individual therapeutics have been determined within ranges.

For administration of collagenase, for example, MMP-1, the dosage can be in the range of about 5 μg to about 50 mg/kg of tissue to which the application is directed, also about 50 μg to about 5 mg/kg, also about 100 μg to about 500 μg/kg of tissue, and about 200 to about 250 ug/kg. When a cAMP-elevating agent is administered in combination with collagenase, for example, MMP-1, the dosage of the cAMP-elevating agent can be in the range of about 5 μg to about 50 μg/kg of tissue to which the application is directed, also about 50 μg to about 5 mg/kg, also about 100 μg to about 500 μg/kg of tissue, and about 200 to about 250 ug/kg. In some instances, a ratio of collagenase, e.g., MMP-1 and cAMP elevating agent is used, such as, e.g., a molar ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 mole:mole. In some embodiments, a weight ratio is used, such as, e.g., a weight ratio of exogenous MMP-1 to cAMP-elevating agent in the range of about 1:1000 to about 1000:1, or about 1:100 to about 100:1, or about 1:25 to about 25:1, or about 1:10 to about 10:1, or about 1:2 to about 2:1 wt:wt.

Administration of the therapeutic combinations of the invention can be accomplished with any combination of the therapeutics, for example, by administering exogenous MMP-1 followed by a cAMP-elevating agent, or by administering a cAMP-elevating agent followed by exogenous MMP-1, or by administering MMP-1 and a cAMP-elevating agent at the same time or in close proximity in time. In addition, if one or more other agents useful in wound treatment are administered, they may be administered before, during, or after treatment with one or both of MMP-1 and/or cAMP. The dosages of each therapeutic for a given wound and a particular patient, are designed to achieve a maximum effective dose for the therapeutic. The dosages appropriate for a given treatment may depend on the particular combination of therapeutics selected for treatment.

The desired dose may be presented in a single dose, as divided doses, or as a continuous infusion. The desired dose can also be administered at appropriate intervals, for example, as two, three, four or more sub-doses per day. One of skill in the art can readily prepare and administer an effective formulation from available information using the teachings provided herein.

The therapeutic agents of the present invention that promote wound healing are suitable for use in the following nonlimiting exemplary situations in which wound healing is required: (1) diabetic foot and leg ulcerations, including neuropathic ulcerations, decubitus lesions, and necrobiosis lipoidica diabeticorum; (2) vascular ulcerations, including venous stasis ulceration, arterial ulcerations, varicose vein ulcerations, post-thrombotic ulcerations, atrophie blanche ulcerations, congenital absence of veins/ulcerations, congenital or traumatic arteriovenous anastomosis, temporal arteritis, atherosclerosis, hypertension (Martorell's ulcerations), thrombosis, embolism, platelet agglutination, ankle blow-out syndrome, or hemangiomas; (3) decubitus ulcers or pressure source (e.g., with bed rest); (4) traumatic ulcerations, such as those caused by external injuries, burns, scalds, chemical injuries, post-surgical injuries, self-inflicted injuries, lesions at an injection site, neonatal or perinatal trauma, or sucking blisters; (5) infestations and bites, such as those caused by spiders, scorpions, snakes, or fly larvae (mydriasis); (6) cold injury, such as pemiosis (erythrocyanosis frigida), or cryoglobulinemic ulcerations; (7) neoplastic ulceration, such as those caused by basal cell carcinomas, squamous cell carcinomas, malignant melanomas, lymphoma, leukemia, Kaposi's sarcoma, tumor erosion, midline lethal granuloma, or Wegener's granulomatosis; (8) blood diseases with ulcerations, such as polycythemia, spherocytosis, or sickle cell anemia; (9) skin diseases with ulcerations, such as tinea, psoriasis, pemphigoid, pemphigus, neurotic excoriations, trichotillomania, erosive lichen planus, or chronic bullous dermatosis of childhood; (10) metabolic disease ulcerations, such as those associated with diabetes mellitus or gout (hyperuricemia); (11) neuropathic ulcerations, such as those associated with diabetes mellitus, tabes dorsalis, or syringomyelia; (12) ischemic ulcerations, such as those associated with scars, fibrosis, or radiation dermatitis; (13) vasculitis ulcerations, such as those associated with lupus erythematosus, rheumatoid arthritis, scleroderma, immune complex disease, pyoderma gangrenosum, or ulceration associated with lipodermatosclerosis; (14) infectious ulcerations, such as: (a) viral ulcerations, e.g. those associated with Herpes simplex or Herpes zoster in an immunocompromised or normal individual; (b) bacterial infections with ulcerations, such as those associated with tuberculosis, leprosy, swimming pool granuloma, ulceration over osteomyelitis, Buruli ulcer, gas gangrene, Meleny's ulcer, bacterial gangrene associated with other bacterial infection (e.g., streptococcal infection), scalded skin syndrome, eethyma gangrenosum (such as can occur in children infected with Pseudomonas aeruginosa), and toxic epidermal necrolysis; (c) mycotic ulcerations, such as those associated with superficial fungal infection or deep fungal infection; (d) spirochetal ulcerations, such as those associated with syphilis or yaws: (e) leishmaniasis; (f) mydriasis; or (g) cellulitis; (15) surgical ulcerations, such as those associated with closed incisions or excisions, open incisions or excisions, stab wounds, necrotic incisions or excisions, skin grafts, or donor sites; or (16) other ulcerations, such as those associated with skin tears (traumatic ulcerations), fistula, peristomal ulcerations, ulcerations associated with aplasia cutis congenita, ulcerations associated with epidermolysis bullosa, ulcerations associated with ectodermal dysplasias, ulcerations associated with congenital protein C or S deficiency, ulcerations associated with congenital erosive and vesicular dermatosis, ulcerations associated with acrodermatitis enteropathica, and amputation stump ulcerations.

The topical therapeutic compositions may also be used orally in the form of a mouthwash or spray to protect or accelerate the healing of oral tissue such as mouth sores, burns, surgical sites, and ulcerations. The topical therapeutic compositions may further be used in opthomological preparations to treat wounds such as those which result from corneal ulcers, radialkeratotomy, corneal transplants, epikaratophakia and other surgically induced wounds in the eye. The thermorectal therapeutic compositions may be used in anorectal creams and suppositories to treat such conditions as pruritus, and proctitis, anal fissures, and hemorrhoids.

In some embodiments, a collagenase, e.g., MMP-1, and a cAMP-elevating agent are administered as part of a wound dressing, either integrally associated with the dressing or as a coating on the dressing. Wound dressings are well-known in the art. These include, without limitation, films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer). See, e.g., Kannon and Garrett, Dermatol. Surg. 21:583-590 (1995); Davies, Burns 10:94 (1983).

In some embodiments, the methods of the invention provide a method of enhanced wound healing by administering to the wounded individual an effective amount of a collagenase, e.g., MMP-1, and, optionally, a cAMP-elevating agent. The a collagenase, e.g., MMP-1, and a cAMP-elevating agent may be administered by any appropriate means; in some embodiments, the a collagenase, e.g: MMP-1, and a cAMP-elevating agent are administered topically, e.g., in a lotion. In some embodiments, an MMP-1 contains a peptide sequence that is at least about 70%, 80%, 90%, or 95% identical to one of SEQ ID NOS: 1, 2, or 3. The concentration of the collagenase, e.g., MMP-1, can be more than about 0.00005%, 0.0001%; 0.001%; 0.01%; 0.1%; 1%, or 10%; the collagenase, e.g., MMP-1 can be at a concentration of less than about 15%, 10%, 1%, 0.1%, 0.01%; 0.001%; or 0.0001%; in some embodiments, about 0.0001% to about 0.01%, in some embodiments about 0.001%. The concentration of the cAMP-elevating agent can be more than about 0.00005%, 0.0001%; 0.001%; 0.01%; 0.1%; 1%, or 10%; the cAMP-elevating agent can be at a concentration of less than about 15%, 10%, 1%, 0.1%, 0.01%; 0.001%; or 0.0001%; in some embodiments, about 0.0001% to about 0.01%, in some embodiments about 0.001%. If applied topically, the lotion containing the collagenase, e.g., MMP-1, and a cAMP-elevating agent is applied at a frequency of once to three times per day, in some embodiments once per day, until the desired result, e.g., healing of the wound, is observed. In some embodiments initial daily application may be followed by topical application once to three times per week, in some embodiments once per week, thereafter, until healing is complete.

In some embodiments, the methods of the invention provide a method of enhanced wound healing in an individual with a wound that results in reduced scarring of the wound site by administering to the wounded individual an effective amount of a collagenase, e.g., MMP-1, and, optionally, a cAMP-elevating agent. The a collagenase, e.g., MMP-1, and a cAMP-elevating agent may be administered by any appropriate means; in some embodiments, the a collagenase, e.g., MMP-1, and a cAMP-elevating agent are administered topically, e.g., in a lotion. In some embodiments, an MMP-1 contains a peptide sequence that is at least about 70%, 80%, 90%, or 95% identical to one of SEQ ID NOS: 1, 2, or 3. The concentration of the collagenase, e.g., MMP-1, can be more than about 0.00005%, 0.0001%; 0.001%; 0.01%; 0.1%; 1%, or 10%; the collagenase, e.g., MMP-1 can be at a concentration of less than about 15%, 10%, 1%, 0.1%, 0.01%; 0.001%; or 0.0001%; in some embodiments, about 0.0001% to about 0.01%, in some embodiments about 0.001%. The concentration of the cAMP-elevating agent can be more than about 0.00005%, 0.0001%; 0.001%; 0.01%; 0.1%; 1%, or 10%; the cAMP-elevating agent can be at a concentration of less than about 15%, 10%, 1%, 0.1%, 0.01%; 0.001%; or 0.0001%; in some embodiments, about 0.0001% to about 0.01%, in some embodiments about 0.001%. If applied topically, the lotion containing the collagenase, e.g., MMP-1, and a cAMP-elevating agent is applied at a frequency of once to three times per day, in some embodiments once per day, until the desired result, e.g., healing of the wound with reduced scarring or scarless healing is observed. In some embodiments initial daily application may be followed by topical application once to three times per week, in some embodiments once per week, thereafter, until healing is complete.

In some embodiments, the methods of the invention provide a method of enhanced healing of a burn wound, by administering to the wounded individual an effective amount of a collagenase, e.g., MMP-1, and, optionally, a cAMP-elevating agent. In some embodiments, the methods of the invention provide a method of enhanced healing of a burn wound that results in reduced or substantially no visible scarring of the wound site, by administering to the wounded individual an effective amount of a collagenase, e.g., MMP-1, and a cAMP-elevating agent. The collagenase, e.g., MMP-1, and cAMP-elevating agent may be administered by any appropriate means; in some embodiments, the collagenase, e.g., MMP-1, and cAMP-elevating agent are administered topically. In some embodiments, an MMP-1 contains a peptide sequence that is at least about 70%, 80%, 90%, or 95% identical to one of SEQ ID NOS: 1, 2, or 3. The methods may utilize the collagenase, e.g., MMP-1, and cAMP-elevating agent neat or in any pharmaceutically acceptable carrier appropriate to topical administration for burn wounds. The concentration of the collagenase, e.g., MMP-1, can be more than about 0.00005%, 0.0001%; 0.001%; 0.01%; 0.1%; 1%, or 10%; the collagenase, e.g., MMP-1 can be at a concentration of less than about 15%, 10%, 1%, 0.1%, 0.01%; 0.001%; or 0.0001%; in some embodiments, about 0.0001% to about 0.01%, in some embodiments about 0.001%. The concentration of the cAMP-elevating agent can be more than about 0.00005%, 0.0001%; 0.001%; 0.01%; 0.1%; 1%, or 10%; the cAMP-elevating agent can be at a concentration of less than about 15%, 10%, 1%, 0.1%, 0.01%; 0.001%; or 0.0001%; in some embodiments, about 0.0001% to about 0.01%, in some embodiments about 0.001%. The collagenase, e.g., MMP-1, and cAMP-elevating agent are applied at a frequency of once to three times per day, in some embodiments once per day, until the desired result, e.g., healing of the burn wound, in some embodiments, with reduced scarring or scarless healing, is observed. In some embodiments initial daily application may be followed by topical application once to five times per week, in some embodiments once per week, or twice per week, or three times per week thereafter, until healing is complete.

In some embodiments, the methods of the invention provide a method of enhanced healing of a chronic wound, by administering to the wounded individual an effective amount of collagenase, e.g., MMP-1, and, optionally, a cAMP-elevating agent. In some embodiments, the methods of the invention provide a method of enhanced healing of a chronic wound in a diabetic individual, by administering to the individual an effective amount of collagenase, e.g., MMP-1, and cAMP-elevating agent. The collagenase, e.g., MMP-1, and cAMP-elevating agent may be administered by any appropriate means; in some embodiments, the collagenase, e.g., MMP-1, and cAMP-elevating agent are administered topically. In some embodiments, an MMP-1 contains a peptide sequence that is at least about 70%, 80%, 90%, or 95% identical to one of SEQ ID NOS: 1, 2, or 3. The methods may utilize the collagenase, e.g., MMP-1, and cAMP-elevating agent neat or in any pharmaceutically acceptable carrier appropriate to topical administration for chronic wounds. The concentration of the collagenase, e.g., MMP-1, can be more than about 0.00005%, 0.0001%; 0.001%; 0.01%; 0.1%; 1%, or 10%; the collagenase, e.g., MMP-1 can be at a concentration of less than about 15%, 10%, 1%, 0.1%, 0.01%; 0.001%; or 0.0001%; in some embodiments, about 0.0001% to about 0.01%, in some embodiments about 0.001%. The concentration of the cAMP-elevating agent can be more than about 0.00005%, 0.0001%; 0.001%; 0.01%; 0.1%; 1%, or 10%; the cAMP-elevating agent can be at a concentration of less than about 15%, 10%, 1%, 0.1%, 0.01%; 0.001%; or 0.0001%; in some embodiments, about 0.0001% to about 0.01%, in some embodiments about 0.001%. The collagenase, e.g., MMP-1, and cAMP-elevating agent are applied at a frequency of once to three times per day, in some embodiments once per day, until the desired result, e.g., healing of the chronic wound, in some embodiments, is observed. In some embodiments where the enhanced wound healing involves halting the spread of a chronic wound, e.g., where the wound does not heal completely or heals completely but very slowly, the application of collagenase, e.g., MMP-1, and cAMP-elevating agent may continue indefinitely. In some embodiments initial daily application may be followed by topical application once to five times per week, in some embodiments once per week, or twice per week, or three times per week thereafter, until healing is complete.

In some embodiments, the collagenase, e.g., MMP-1, and, optionally, a cAMP-elevating agent are used in combination with another wound care method (e.g., for debridement of burns, or the like) or agent. If the collagenase, e.g., MMP-1, and cAMP-elevating agent are used in combination with another wound care method or composition, any combination of the collagenase, e.g., MMP-1, and cAMP-elevating agent and the additional method or composition may be used. Thus, for example, if use of collagenase, e.g., MMP-1, and cAMP-elevating agent is in combination with another wound care agent, the two may be administered simultaneously, consecutively, in overlapping durations, in similar, the same, or different frequencies, etc. In some cases a composition will be used that contains collagenase, e.g., MMP-1, and cAMP-elevating agent in combination with one or more other wound care agents.

Combinations

The compositions and methods of the invention may also be used in combination with other substances, compositions, or methods that enhance wound healing or that ameliorate side effects of wounds and wound healing, such as pain and inflammation. Substances of use in the invention include, but are not limited to, initiators and enhancers of wound healing, antiinflammatory agents, antimicrobial agents and antiseptics, antiviral substances, antipruritics, anesthetic or analgesic compounds, vitamins and antioxidants, and the like. These factors may be used topically, systemically, or both, as appropriate for the treatment of the particular wound. Substances of use in combination with the therapeutic compositions of the invention are detailed in the section describing Compositions, above.

C. Cancer Therapeutics

Another embodiment encompasses a method of inhibiting endocytosis and migration of cells comprising administering a collagenase, e.g., MMP-1, optionally in combination with a cAMP-enhancing agent to an organism. It is contemplated that inhibition of endocytosis or cell migration may prevent the invasiveness of cancerous cells.

Thus, in some embodiments the methods of the invention encompass treating an individual for cancer by administering to the individual an effective amount of a collagenase, e.g., MMP-1, optionally in combination with a cAMP-enhancing agent. The invention also encompasses compositions for the treatment of cancer that include a collagenase, e.g., MMP-1, optionally in combination with a cAMP-enhancing agent. In some embodiments, the composition is suitable for topical application. Any type of cancer may be treated, but the compositions of the invention are especially suited to the topical treatment of skin cancers, e.g., basal cell carcinoma, squamous cell carcinoma, and malignant melanoma.

In addition, precancerous conditions may be treated using the compositions of the invention. Actinic keratoses, for example, are superficial inflammatory premalignant tumors arising on sun-exposed and irradiated skin. The lesions are erythematous to brown with variable scaling. Current therapies include excisional and cryosurgery. These treatments are painful, however, and often produce cosmetically unacceptable scarring. Accordingly, treatment of keratosis, such as actinic keratosis, can include application, preferably topical, of a collagenase, e.g., MMP-1, optionally in combination with a cAMP-enhancing agent in amounts sufficient to inhibit hyperproliferation of epidermal/epidermoid cells of the lesion, optionally in conjunction with other active ingredients.

Basal cell carcinoma, squamous cell carcinoma, papilloma, and keratoacanthoma may also be treated by the methods of the invention. The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction, “squamous cell carcinoma”, which refers to carcinomas arising from squamous epithelium and having cuboid cell. “Keratoacanthoma” is a relatively common low-grade malignancy that originates in the pilosebaceous glands and closely and pathologically resembles squamous cell carcinoma. Another carcinomatous epithelial growth is “papillomas”, which refers to benign tumors derived from epithelium and having a papillomavirus as a causative agent. All of these carcinomas may be treated by methods of the invention by administering an effective amount of a collagenase, e.g., MMP-1, in combination with a cAMP-enhancing agent to the affected individual, typically as a topical preparation. Treatment may be in combination with other treatments as are known in the art.

In addition, malignant melanoma may be treated by methods of the invention. Methods of the invention include treatment of an individual suffering from malignant melanoma by administering to the individual an effective amount of a collagenase, e.g., MMP-1, in combination with a cAMP-enhancing agent, alone or in combination with other agents known in the art for the treatment of malignant melanoma.

D. Cosmetic Applications

The invention further provides methods, compositions, and kits for the cosmetic use of a collagenase, e.g., MMP-1, optionally in combination with a cAMP-enhancing agent.

In some embodiments, the invention provides a method for cosmetic treatment of skin in an individual that includes topically administering to an individual desiring or in need of cosmetic treatment an effective amount of a collagenase, e.g., MMP-1, optionally in combination with a cAMP-enhancing agent. In embodiments of methods of the invention, the a collagenase, e.g., MMP-1, optionally in combination with a cAMP-enhancing agent are administered at an average frequency of about once per day to about five times per day. In some embodiments, the a collagenase, e.g., MMP-1, optionally in combination with a cAMP-enhancing agent are administered at an average frequency of about once per day to about once per week. In some embodiments, the a collagenase, e.g., MMP-1, optionally in combination with a cAMP-enhancing agent are administered at an average frequency of less than about once per day.

In embodiments of methods of the invention, the a collagenase, e.g., MMP-1, optionally in combination with a cAMP-enhancing agent are applied in a vehicle such as, e.g., a spray, ointment, gel, lotion, milk, liposomal preparation, or patch. In some embodiments, the vehicle is a lotion. In some embodiments where the vehicle is a lotion, the lotion contains the a collagenase, e.g., MMP-1, optionally in combination with a cAMP-enhancing agent at a total concentration of about 0.0001% to about 10% by weight.

In some embodiments of the methods of the invention, the a collagenase, e.g., MMP-1, optionally in combination with a cAMP-enhancing agent are administered in combination with one or more other cosmetic or dermatological agents. In some embodiments, the other cosmetic or dermatological agent or agents is hydroxy acids, retinoic acid derivatives, free radical scavengers, botulinum toxin, sunscreens, anti-acne agents, or anticellulite agents.

In another aspect, the invention provides cosmetic compositions. In some embodiments of this aspect, the compositions include a collagenase, e.g., MMP-1, optionally in combination with a cAMP-enhancing agent in a cosmetically acceptable vehicle at a concentration of greater than about 0.00005% by weight. In some embodiments of this aspect, the compositions include a collagenase, e.g., MMP-1, optionally in combination with a cAMP-enhancing agent at a total concentration of about 0.0001% to about 10% by weight.

A wide variety of additional components may be added to the compositions of the present invention, as long as the components are selected so as to avoid any undesirable reaction with the primary components (e.g., one or more of the sunscreen agents) of the composition. The CTFA Cosmetic Ingredient Handbook, Seventh Edition, 1997 and the Eighth Edition, 2000 (incorporated by reference herein), provide a broad source of possible cosmetic and pharmaceutical ingredients typically used in skin care compositions. Examples of such additional components include one or more of the following: Absorbents, abrasives, anticaking agents, antifoaming agents, binders, biological additives, buffering agents, bulking agents, chelating agents/sequestrants (e.g., disodium EDTA), chemical additives, colorants, cosmetic astringents, cosmetic biocides, denaturants, drug astringents, emollients (including glycerin aloe Vera, and Vitamins A, C, and D [hydrating agents and skin protectants]), foam boosters, fragrance components, gums, humectants/moisturizers (including urea, guanidine, glycolic acid, polyhydroxy alcohols such as sorbitol, glycerin, hexanetriol, propylene glycol, hexylene glycol and the like, polyethylene glycol, sugars and starches, sugar and starch derivatives, D-panthenol, hyaluronic acid, lactamide monoethanolamine, acetamide monoethanolanine, and mixtures thereof), hydrotropes, neutralizing agents, opacifying agents and pigments, pH adjusters, plasticizers, preservatives, propellants, reducing agents, skin bleaching agents, skin protectants, solubilizing agents, and suspending agents (e.g., Carbomer 1382).

VI. Kits

The present invention further provides kits. Kits include kits for cell culture, therapeutics, or cosmetic applications

A. Cell Culture Kits

In some embodiments, the invention provides kits in which culture medium containing a collagenase, e.g., MMP-1, in combination with a cAMP-enhancing agent is packaged for transport, storage and/or use by a consumer. Such packaging of tissue culture medium for transport, storage, and use is well-known in the art. Packaged medium may include further components for the dispensing and storage of the medium, and may also include separately packaged diluent for dilution of concentrated medium, optional additional ingredient for inclusion by the user if desired, instructions for use, and the like.

In some embodiments of the kits of the invention, supplements for use in tissue culture medium are provided. In one such embodiment, a kit may contain an exogenous MMP-1 is provided in one vial, and a cAMP-elevating agent is provided in another vial so that the end-user may determine the exact ratio to be added to his or her tissue culture medium depending on cell type, stage of culture, desired outcome, and the like. The MMP-1 and cAMP-elevating agent can be provided in a dry powder form, in solid form (i.e., lyophilized), in solution, or in suspension. To the proteins may have been added emulsifiers, salts, preservatives, other proteins, nucleic acids, protease inhibitors, antibiotics, perfumes, polysaccharides, adhesive agents, polymers, microfibrils, oils, etc.

B. Therapeutics

The invention also encompasses kits for therapeutics, such as for wound healing and cancer therapeutics.

In some embodiments, the invention provides a kit for the treatment of wounds in a subject, comprising MMP-1, a cAMP-elevating agent, and instructions for using the MMP-1 and cAMP-elevating agent to treat wounds in the subject. In some embodiments, the subject is a patient with diabetes. In other embodiments, the subject is a burn patient. In still further embodiments, the wound is a chronic wound. In still further embodiments, the wound is a traumatic wound, e.g., a surgical incision. In some embodiments the MMP-1 and the cAMP-elevating agent are supplied as a dressing or part of a dressing for a wound. In some embodiments, the MMP-1 and the cAMP-elevating agent are supplied in one or more compositions, e.g., a gel, salve, cream, lotion, and the like, which may contain the MMP-1 and the cAMP-elevating agent separately or in combination. In these embodiments, the kit may further include one or more dressings for use with the composition(s). In some embodiments, the kit contains a plurality of dressings, where the dressings may be the same size or of various sizes. Dressings, whether containing compositions of the invention or separately packaged, may be sterilely packaged. Compositions containing MMP-1, and/or cAMP-elevating agents, may also be sterilely packaged. The kits may optionally further contain other components, such as gloves, scissors, tape, implements for disposal of used bandages and other waste, masks, antiseptic, antibiotics, and the like.

C. Cosmetics

The invention also provides kits for use in the cosmetic treatment of skin. In some embodiments of this aspect of the invention, the kit includes a composition containing a collagenase, e.g., MMP-1, in combination with a cAMP-enhancing agent in a cosmetically acceptable vehicle and instructions for use of the composition in the cosmetic treatment of skin.

VII. Business Methods

In one aspect, the invention provides business methods. In one embodiment, the invention provides a method of doing business comprising marketing and sale of cell culture media by supplying a composition comprising isolated collagenase and a cAMP-elevating agent to a customer, and receiving payment for the composition. In some embodiments, the composition is a cell culture medium. In some embodiments, the composition is a kit comprising a first container containing isolated collagenase and a second container containing a cAMP-elevating agent. In some embodiments, the sample is from an individual. In some embodiments the cell culture media is media for the culture of keratinocytes. In some embodiments the cell culture media is media for the culture of chondrocytes. In some embodiments the cell culture media is media for the culture of fibroblasts. In some embodiments the cell culture media is media for the culture of hepatocytes

In some embodiments, the invention provides a method of doing business by providing a health care provider, or a supplier to a health care provider, with a therapeutic composition comprising collagenase and, optionally, a cAMP-elevating agent, and receiving payment for the composition. In some embodiments, the composition is marketed directly to the consumer or individual in need thereof. In some embodiments, the composition is a bandage. In some embodiments, the composition is a cream, gel, lotion, ointment, or spray.

### EXAMPLES

### Example 1

### Methods

This Example presents general Materials and Methods generally used in the Examples presented herein.

Isolation and Culture of Human Keratinocytes

Media and reagents were obtained from Invitrogen Corp., Sigma, Roche Life Sciences, Serva Electrophoresis, Cascade Biologicals, and Cambrex Bio Sciences. Human neonatal/adult skins, mouse fetal/newborn/adult skin, rat fetal/newborn/adult skin, were placed in serum-free medium (SFM) without growth factors containing 5 μg/ml gentamycin and were stored at 4° C. Skins were briefly rinsed in Dulbecco's phosphate-buffered saline (DPBS), without Ca⁺⁺ and Mg⁺⁺, containing 20 μg/ml gentamycin for 60 minutes. Skins were then cut into small pieces and the pieces were transferred, to a petri dish containing 0.15% dispase and 0.5% collagenase, and were incubated 30 min-2 hours at 37° C. with gentle mixing to aid in tissue dissociation. Pooling of the tissue specimens was performed to reduce the effects of donor-to-donor growth variation. The cell suspension was transferred to a sterile centrifuge tube and the cells pelleted by centrifugation at 50×g (500 rpm) for 5 minutes at 22° C., and washed three times with DMEM. The supernatant was discarded, the cell pellet resuspended in the appropriate medium, and cell densities determined using a hemacytometer. Cells were plated in culture flasks or dishes.

Secondary cultures were established by removing the spent medium, briefly washing the cell monolayer with DPBS and adding an appropriate volume of recombinant trypsin (Invitrogen Corp). Cells were incubated at room temperature, until they detached (about 15-30 minutes) from the culture surface. Trypsin activity was inactivated by addition of 10 mg/ml soybean trypsin inhibitor solution; cells were pelleted by centrifugation at 50×g (500 rpm) for 5 minutes at 22° C., washed once with SFM, and resuspended in the appropriate medium. Secondary cell cultures were also established from primary keratinocytes obtained from Cambrex and Cascade Biologicals, with results comparable to those found with cultures established from normal human neonatal foreskins.

Trypsinization times are important to the performance of any keratinocyte medium. Human keratinocytes that remain in trypsin too long have lower plating efficiencies and may be induced to differentiate.

Cultures were incubated at 37° C. in a humidified atmosphere consisting of 5% CO₂/95% air. Stock cultures were maintained at a split ratio of 1:2 to 1:3 and subcultured at 70% to 80% confluence. Keratinocytes at passage 0 through passage 4 were used for experimental evaluation.

Conventional Protocol for Processing Human Neonatal Foreskins, Human Adult Skin, Newborn and Adult Murine Skin Samples.

The samples were placed in serum-free medium (SFM) without growth factors containing 5 μg/ml gentamycin and stored at 4° C. Skins were briefly rinsed in 70% isopropanol and then placed into Dulbecco's phosphate-buffered saline (DPBS), without Ca.sup.++ and Mg.sup.++, containing 20 μg/ml gentamycin for 60 minutes. Skins were then cut into halves or quarters, depending upon the size of the tissue, and the pieces transferred, dermis side down, to a petri dish containing 25 units/ml dispase, and incubated 18-24 hours at 4° C. Epidermal sheets were separated from the full-thickness skin with forceps, pooled in 60 mm culture dishes containing 5-7 ml of 0.05% trypsin/0.53 mM EDTA, and incubated at 37° C. for 15-20 minutes with gentle pipetting to aid in tissue dissociation. Pooling of the tissue specimens was performed to reduce the effects of donor-to-donor growth variation. Trypsin activity was terminated by addition of soybean trypsin inhibitor (10 mg/ml in DPBS). The cell suspension was transferred to a sterile centrifuge tube and the cells pelleted by centrifugation at 50×g for 5 minutes at 22° C., and washed once with SFM. The supernatant was discarded, the cell pellet resuspended in the appropriate medium, and cell densities determined using a hemacytometer. Cells are pelleted in culture flasks or dishes.

Cultures were incubated at 37° C. in a humidified atmosphere consisting of 5% CO₂/95% air. Stock cultures are maintained at a split ratio of 1:2 to 1:3 and subcultured at 70% to 80% confluence. Keratinocytes at passage 0 through passage 4 are used for experimental evaluation.

A specific protocol for fetal or adult keratinocytes and fibroblasts is given below.

Isolation of Fetal or Adult Keratinocytes & Fibroblasts from Skin Samples from Human, Mouse and Rat.

Removed the skin under aseptic conditions. Placed the tissue into complete keratinocyte medium or DMEM containing Gentamicin at a concentration of 5-10 μg/ml. The tissue may be process or stored at 4° C. for up to 1 week. Trimmed the tissue with scalpel No. 10. Digested the tissue in Dispase at final concentration of 1.5% and Collagenase at 0.5%. Incubated the tissue for 30 min (fetal) to up to 2 hrs (adult tissue) at 37° C. After incubation pipeted up and down with a 10 ml pipet to dissociate the cells. Passed the digested tissue through 100 μm cell strainer. Centrifuged the cells at 500 rpm for 10 minutes at 4° C. Washed the cells three times into DMEM or DPBS at 500 rpm for 5 minutes each. Re-suspended the cells in complete * keratinocyte medium (*see complete formulation) The cells were seeded in coated (collagen I or collagen IV) or uncoated flasks at a density of 3 to 5×10⁴ per cm². Incubated the cells at 37° C. in a humidified 5% CO₂ in air. 24 hrs later removed the keratinocyte medium and the unattached cells (most were fibroblasts), centrifuged and re-suspended the cells into the *fibroblast medium (*see formulation, below). Rinsed twice the keratinocytes with D-PBS (without Ca and Mg) to removed 95 to 99% of the fibroblasts. Added fresh complete *keratinocyte medium. The culture may reach 60 to 70% confluence in 3 to 5 days following isolation and setup. Keratinocyte cultures were medium changed every other day after the first 24 hrs until the cells reach 60% to 70% confluence after which time keratinocytes were subcultured.

### Example 2

### Formulation of Complete Medium, General Procedure

Formulation of Basal Cell Culture Medium. Basal Media and reagents were obtained from Invitrogen Corp., Sigma, Roche Life Sciences, Serva Electrophoresis, Cascade Biologicals, Cambrex Bio Sciences. Growth Supplement was added according to manufacturer's instructions including, human insulin, human transferrin, hydrocorisone, EGF, FGF-1, Heparin, Epinephrine. In some cases, additional ingredients included BPE, FBS, BSA, FCS, Lipids, or other animal derived components. A stock solution of forskolin (1 mg/ml) was prepared in 100% Ethanol added to the above to fully supplement the media. A stock solution of collagenase (1 mg/ml) was prepared in DPBS, and added to the above to fully supplement the media. The complete medium was used immediately or stored at 4° C. under diminished light conditions until use.

### Example 3

### Formulation of Complete Keratinocyte Medium

MCDB 153 was supplemented with the following ingredients: Insulin at a concentration of about 5 μg/ml. Transferrin at 10 μg/ml. Hydrocortisone at 0.1-0.2 μg/ml EGF at 0.2 ng/ml FGF-1 at 5 ng/ml Heparin at 10-15 USP/L Collagenase from Clostridium histolyticum at about 2-3.5 ug/ml OR rhMMP-1 at about 1.5-2 ug/ml Forskolin at about 1.5-2.5 ug/ml. Alternative formulations may include the above factors plus BPE (Bovine Pituitary Extract) at about 10-15 μg/ml

### Example 4

### Formulation of Complete Fibroblast Medium

DMEM high Glucose from Invitrogen was supplemented with: FBS (Fetal calf serum) at 2-5% Collagenase from C.H. at 10-15 μg/ml. Insulin, Transferrin, Hydrocortisone, EGF and/or others factors were optionally added at the concentrations given for keratinocyte medium.

### Example 5

### Evaluation of Shelf Life of Media Containing Collagenase and Forskolin

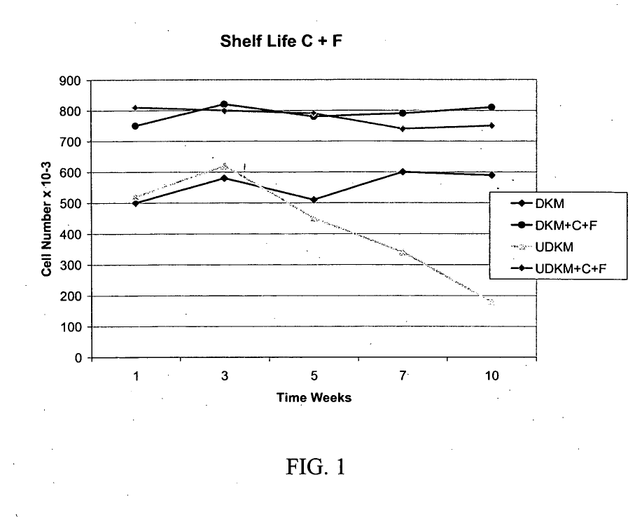

To evaluate the shelf life of the medium of the present invention, primary human keratinocytes were cultivated in the media from supplier A (Defined Keratinocyte-Media) and in media from Supplier B (Undefined Keratinocyte-Media), with and without collagenase and forskolin. Media were evaluated over a storage period of 10-weeks after formulation, and cell counts were compared at each time point to those obtained from freshly prepared media. As shown in FIG. 1, the defined media (supplier A), and the Undefined Keratinocytes media (supplier B) had a shelf life of over 10 weeks when fully supplemented with collagenase and forskolin. These results indicate that the medium of the present invention, when stored properly as described above, demonstrates an extended shelf life compared to more traditionally used BPE-containing media.

### Example 6

### Effects of Collagenase on the Survival of the Cells in Depleted Media

To evaluate the activity of the fully supplemented medium of the present invention, primary human keratinocytes were cultivated for eight days in the media from supplier A (Defined Keratinocyte-Media) and in media from Supplier B (BPE-containing media), with and without collagenase and forskolin. Cells were cultured and evaluated over a week period after formulation was added without media changes during said period. Cell counts were compared to those obtained from supplier A and B without collagenase.

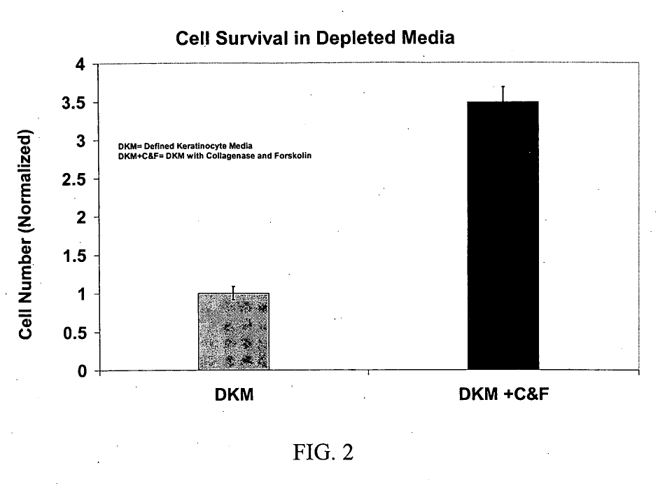

As shown in FIG. 2, the defined media (supplier A), and the Undefined Keratinocytes media (supplier B) of the present invention had an extended activity and survival over 8 days when fully supplemented with collagenase and forskolin, compared to defined media (supplier A), and the Undefined Keratinocytes media (supplier B) without collagenase.

### Example 7

### Effects of Collagenase on the Life Span of Cells

To evaluate the life of the cells in the medium of the present invention, primary human keratinocytes were cultivated in the media from supplier A (Defined Keratinocyte-Media) with and without collagenase and forskolin.

All determinations were made in triplicate. Cultures of keratinocytes were established by plating 3,000 trypan blue-excluded cells/cm² in 6 wells plates in the indicated medias. The cultures were incubated at 37° C./95% air/5% CO₂. When cultures reached approximately 75-80% confluence, the cells were harvested using recombinant trypsin and the total number of cells determined. Subsequent cultures were established by pooling cells from each set of triplicate cultures at a density of 3,000 trypan blue-excluded cells/cm² in 6 wells (9.5 cm²) plates in the indicated medias. The populations doublings (y) achieved during each culture interval (passage) were calculated as 2^{y}=the fold increase in cell number during each passage.

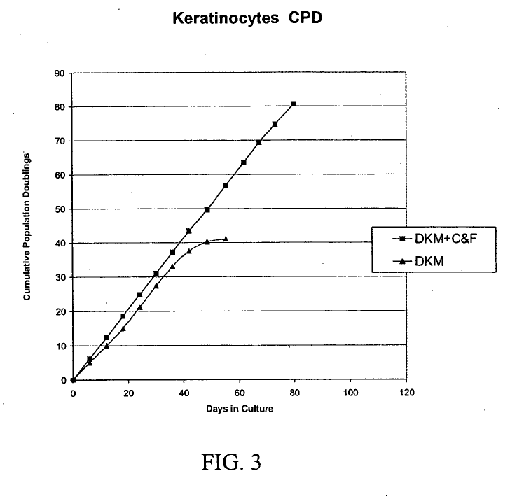

As shown in FIG. 3, the defined media supplemented with collagenase and forskolin of the present invention had an extended life span, with over 80 populations doublings when fully supplemented with collagenase and forskolin compared to defined media (supplier A) without collagenase and forskolin.

### Example 8

### Normal Human Articular Chondrocytes (NHAC) and Normal Rodent Chondrocytes

Currently, there is not any SFM commercially available for chondrocytes. Using the serum-containing media available, the life-span of the chondrocytes in culture is very limited (up to 3 to 4 passages, between 10-15 doublings times). Thus, the present example illustrates the use of collagenase and forskolin to allow the used of SFM with chondrocytes.

Normal human adult chondrocytes and normal rodent chondrocytes were cultured in a SFM. Collagenase (5 μg/ml) was added into the DMEM serum free media (SFM) and tested on said primary and secondary chondrocytes cultures.

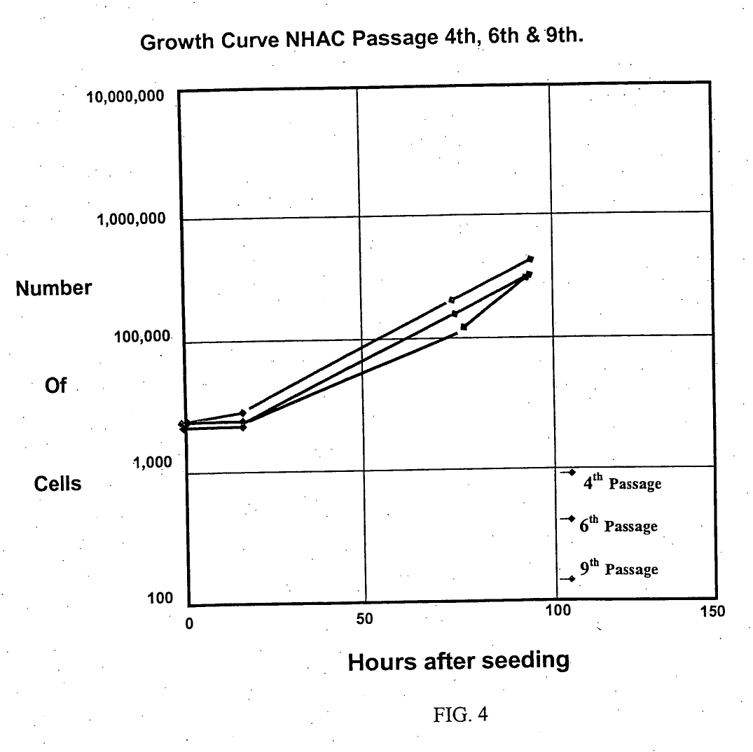

FIG. 4 shows that, even after 4, 6, and 9 passages, chondrocytes demonstrated robust growth in the culture media of the invention.

This Example illustrates that media supplemented with collagenase and forskolin is able to support proliferation and increase the life-span of human and rodent chondrocytes. In further experiments, up to 11 passages has been reached (approximately 40-45 doublings times).

### Example 9

### Normal Human Dermal Fibroblasts

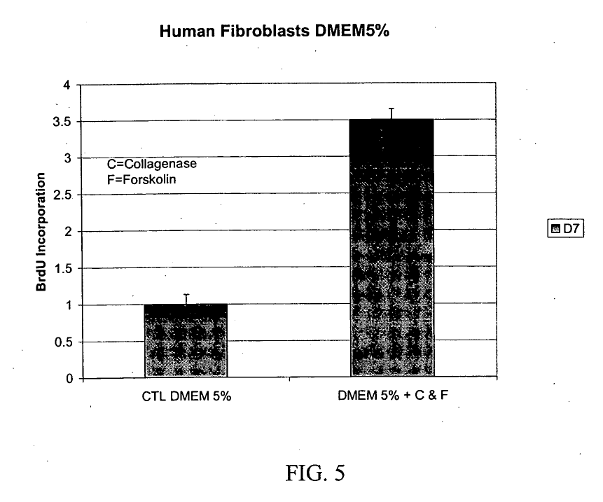

Human dermal adult and newborn skin fibroblasts were cultured in a SFM and serum-containing media (DMEM 5% FBS). Collagenase (1 μg/ml) was added to the DMEM serum free media (SFM) and tested on primary and secondary culture of human adult and newborn fibroblasts. In both cases (SFM and DMEM 5%) Collagenase increase cell proliferation up to 2-2.5 fold over the controls. See FIG. 5.

### Example 10

### Secondary Culture of Rat Hepatocytes

Collagenase and forskolin (2 μg/ml) were added to the serum free media (SFM) for hepatocytes from Cambrex and tested on secondary culture of rat hepatocytes. Cambrex serum free media (SFM) reached up to 2 passages in 5 weeks. However, added collagenase and forskolin to Cambrex SFM media increased the proliferation and number of passages reaching up to 4 passages in the same period of time.

### Example 11

### Accelerated Re-Epithelialization in Mouse Excision Wounds

Wound healing in mammals proceeds by a series of overlapping highly coordinated events. Dermal wound repair commences with the arrest of hemorrhage followed by an inflammatory response, re-epithelialization of the wound, and formation of granulation tissue within the wound space, culminating in the production of a scar. In order to study the processes a rodent model was used utilizing full thickness excisional dermal wounds, which allow for macroscopic observations.

One full-thickness disk of skin (1×1 cm) was removed from the backs of 20 mice. A thin layer of collagenase (low endotoxin bacterial collagenase, 120 U) mixed with forskolin (12.5 mg) per gram of white petrolatum USP was applied onto the wound immediately after the excision wound and every day for 15 days. White petrolatum USP (WP USP) was used as control.

The topical application of collagenase and forskolin accelerated re-epithelialization in mouse excision wounds. 100% re-epithelialization was observed in collagenase+forskolin treated wounds vs. approximately 70% re-epithelialization observed in control wounds (WP USP only) on day 15).

This Example illustrates that application of collagenase and a cAMP-elevating agent to a wound accelerates wound healing.

What is claimed is:

1. A cell culture medium comprising keratinocyte culture medium, collagenase and a cyclic adenosine monophosphate (cAMP)-elevating agent, wherein the collagenase and the cAMP-elevating agent are not produced by cells in the culture medium.

2. A cell culture medium comprising keratinocyte culture medium and matrix metalloprotease (MMP) wherein the MMP is not produced by cells in the medium, and wherein the MMP is selected from the group consisting of MMP-1, MMP-2, MMP-8, and MMP-9.

3. The cell culture medium of claim 1 wherein the collagenase is low-endotoxin collagenase.

4. The cell culture medium of claim 2 wherein the MMP is MMP-1

5. The medium of claim 2 further comprising a cAMP-elevating agent.

6. The cell culture medium of claim 5 wherein the cAMP-elevating agent increases intracellular cAMP levels through interactions with cellular G-proteins.

7. The cell culture medium of claim 5 wherein the cAMP-elevating agent increases intracellular cAMP levels through directly increasing cAMP levels.

8. The cell culture medium of claim 5 wherein the cAMP-elevating agent increases intracellular cAMP levels through inhibition of a cAMP phosphodiesterase.

9. The cell culture medium of claim 1 or 5 wherein the cAMP-elevating agent is selected from the group consisting of forskolin, cholera toxin, dibutyryl cAMP, isobutylmethylxanthine, theophylline, isoproterenol, and PGE2.

10. The cell culture medium of claim 9 wherein the cAMP-elevating agent is forskolin.

11. The cell culture medium of claim 2 wherein the MMP-1 is recombinant.

12. The cell culture medium of claim 2 wherein the MMP-1 is at a concentration of between about 0.1 ug/ml and 10 ug/ml.

13. The cell culture medium of claim 2 wherein the MMP-1 is at a concentration of between about 1 ug/ml and 3 ug/ml.

14. The cell culture medium of claim 3 wherein the forskolin is at a concentration of between about 0.1 ug/ml and 10 ug/ml.

15. The cell culture medium of claim 3 wherein the MMP-1 is at a concentration of between about 0.1 ug/ml and 10 ug/ml and the forskolin is at a concentration of between about 0.1 ug/ml and 20 ug/ml.

16. The cell culture medium of claim 1, 2, or 5 wherein the medium is serum-free.

17. The cell culture medium of claim 16 wherein the medium comprises bovine pituitary extract.

18. The cell culture medium of claim 1, 2, or 5 wherein the medium is a defined medium.

19. The cell culture medium of claim 17 wherein the bovine pituitary extract is present at a concentration of about 10-15 ug/ml.

20. The cell culture medium of claim 1, 2, or 5 wherein the medium is animal-protein-free.

21. The cell culture medium of claim 9, further comprising insulin, transferrin, and hydrocortisone.

22. The cell culture medium of claim 21, wherein the MMP-1 is at a concentration of between about 0.1 ug/ml to 10 ug/ml and the forskolin is at a concentration of between about 0.1 ug/ml and 20 ug/ml.

23. The cell culture medium of claim 21, wherein the MMP-1 is at a concentration of about 1 to 5 ug/ml and the forskolin is at a concentration of about 0.8-2.0 ug/ml.

24. A cell culture medium comprising keratinocyte culture medium and collagenase, wherein the collagenase is not produced by cells growing in the medium, and wherein the collagenase is low-endotoxin collagenase.

25. The cell culture medium of claim 1 or 24 wherein the collagenase is present at a concentration of about 0.1 to 10 ug/ml.

26. The cell culture medium of claim 1 or 24 wherein the collagenase is present at a concentration of about 0.0001 to 0.05 U/ml.

27. The cell culture medium of claim 1 or 24 wherein the collagenase is bacterial collagenase.

28. The cell culture medium of claim 27 wherein the collagenase is isolated from Clostridium histolyticum.

29. The cell culture medium of claim 28 wherein the collagenase comprises collagenase I.

30. The cell culture medium of claim 28 wherein the collagenase comprises collagenase II.

31. The cell culture medium of claim 28 wherein the collagenase is highly purified.

32. The cell culture medium of claim 23 wherein the MMP-1 is at a concentration of about 1.5-2.0 ug/ml the forskolin is at a concentration of about 1.5-2.0 ug/ml, the insulin is at a concentration of about 2-15 ug/ml, the transferrin is at a concentration of about 5-15 ug/ml, and the hydrocortisone is at a concentration of about 0.02-0.5 ug/ml.

33. The cell culture medium of claim 32 wherein the MMP-1 is recombinant MMP-1.

34. A keratinocyte culture medium comprising collagenase and forskolin, wherein neither the collagenase nor the forskolin is produced by cells in the culture medium.

35. The keratinocyte culture medium of claim 34 further comprising insulin, transferrin, and hydrocortisone.

36. The keratinocyte culture medium of claim 34 or 35 wherein the collagenase is collagenase isolated from Clostridium histolyticum.

37. The keratinocyte culture medium of claim 34 wherein the collagenase is present at about 2-3.5 ug/ml and the forskolin is present at about 1.5-2.5 ug/ml.

38. The keratinocyte culture medium of claim 35 wherein the collagenase is present at about 2-3.5 ug/ml and the forskolin is present at about 1.5-2.5 ug/ml.

39. The keratinocyte culture medium of claim 38 wherein the insulin is present at about 5 ug/ml, the transferrin is present at about 10 ug/ml, and the hydrocortisone is present at about 0.1-0.2 ug/ml.

40. The keratinocyte culture medium of claim 39 further comprising bovine pituitary extract at a concentration of about 10-15 ug/ml.

41. A keratinocyte culture medium comprising a peptide that is at least about 80% identical to SEQ ID NO: 1, 2, or 3, wherein the peptide is not produced by cells in the medium.

42. The keratinocyte culture medium of claim 41 further comprising a cAMP-elevating agent.

43. The medium of claim 41 or 42 wherein the peptide is present at a concentration of about 0.5-5.0 ug/ml.

44. The medium of claim 42 wherein the cAMP-elevating agent is present at a concentration of about 0.5-5.0 ug/ml.

45. The medium of claim 1 that is 10× concentrated.

46. A kit for keratinocyte culture comprising a first container that comprises isolated collagenase and a second container comprising a cAMP-elevating agent.

47. The kit of claim 46 wherein the first and second containers are the same.

48. The kit of claim 46 wherein the collagenase is MMP-1.

49. The kit of claim 46 wherein the isolated collagenase is in aqueous solution.

50. The kit of claim 46 wherein the isolated collagenase is in solid form.

51. The kit of claim 46 further comprising a keratinocyte to be cultured.

52. The kit of claim 46 further comprising instructions.

53. A method of preparing a keratinocyte culture medium comprising adding isolated MMP-1 to a base keratinocyte culture medium.

54. The method of claim 53 further comprising adding to the medium a cAMP-elevating agent.

55. A method of culturing a keratinocyte comprising contacting the keratinocyte with a keratinocyte culture medium that comprises MMP-1, wherein the MMP-1 is not produced by cells in the medium, and culturing the keratinocyte under conditions suitable to support culture of the keratinocyte.

56. The method of claim 55 wherein the medium further contains a cAMP-elevating agent.

57. A method for producing a keratinocyte product comprising:
(a) culturing keratinocytes in the cell culture medium of claim 1 or 2 until said product accumulates; and
(b) recovering said product.

58. The method of claim 57 further comprising purifying said product.

59. A business method for the marketing and sale of keratinocyte culture media comprising supplying a keratinocyte cell culture composition comprising isolated collagenase and a cAMP-elevating agent to a customer, and receiving payment for the composition.

60. A method of culturing keratinocytes comprising contacting growing keratinocytes with a culture medium comprising collagenase, wherein the collagenase is not produced by cells in the medium, and wherein the collagenase is low-endotoxin collagenase.

61. The method of claim 60 wherein the collagenase comprises isolated MMP-1.

62. The method of claim 60 wherein the collagenase comprises recombinant MMP-1.

63. The method of claim 60 wherein the culture medium further comprises a cyclic adenosine monophosphate (cAMP)-elevating agent, wherein the cAMP-elevating agent is not produced by cells in the medium.

64. The method of claim 63 wherein the cAMP-elevating agent is selected from the group consisting of forskolin, cholera toxin, dibutyryl cAMP, isobutylmethylxanthine, theophylline, isoproterenol, and PGE2.

65. The method of claim 64 wherein the cAMP-elevating agent is forskolin.

66. The method of claim 65 wherein the MMP-1 is at a concentration of between about 0.5 ug/ml and 5 ug/ml in the culture medium and the forskolin is present at a concentration of about 0.8 ug/ml to about 5 ug/ml.

67. The method of claim 60 wherein the growing keratinocytes are fetal keratinocytes.

68. The method of claim 60 wherein the culture medium is partially or completely replaced periodically

69. The method of claim 68 wherein the culture medium is partially or completely replaced about every day.

70. The method of claim 68, wherein the keratinocytes are cultured until a cell culture is produced wherein the cells of the cell culture comprise greater than about 99% keratinocytes.

71. The method of claim 54, wherein step (b) is repeated until a cell culture is produced wherein the cells of the cell culture comprise greater than about 99.9% keratinocytes.

72. The method of claim 60 further comprising passing the keratinocytes when they are about 60-70% confluent.

73. A keratinocyte culture that persists for more than about one year, wherein the cells of the culture comprise at least about 99% keratinocytes, and wherein the cells are contacted with cell culture medium comprising collagenase, wherein the collagenase is not produced by cells in the medium.


SEQUENCE LISTING (3 sequences)

SEQ ID NO: 1 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 2 - protein, 450 aa, Homo sapiens
FPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 3 - protein, 369 aa, Homo sapiens
VLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN